SEQUENCE LISTING (44 sequences)

SEQ ID NO: 1 - protein, 412 aa, Artificial Sequence
  exemplary ASS-1 enzyme domain
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 2 - protein, 354 aa, Artificial Sequence
  exemplary OTC enzyme domain
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 3 - protein, 263 aa, Artificial Sequence
  exemplary GD2 target binding moiety
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSS

SEQ ID NO: 4 - protein, 248 aa, Artificial Sequence
  exemplary CD33 target binding moiety
GSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 5 - protein, 241 aa, Artificial Sequence
  exemplary mesothelin target binding moiety
MQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 6 - protein, 221 aa, Artificial Sequence
  exemplary EGFRVIII target binding moiety
QVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDY

SEQ ID NO: 7 - protein, 42 aa, Artificial Sequence
  exemplary 4-1BB intracellular signalling region
KRGRKKLLYIFKQPFMRPVQTTQEEDGCSCRFPEEEEGGCEL

SEQ ID NO: 8 - protein, 36 aa, Artificial Sequence
  exemplary OX-40 intracellular signalling region
RDQRLPPDAHKPPGGGSFRTPIQEEQADAHSTLAKI

SEQ ID NO: 9 - protein, 107 aa, Artificial Sequence
  exemplary CD28 intracellular signalling region with transmembrane domain
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 10 - protein, 38 aa, Artificial Sequence
  exemplary ICOS intracellular signalling region
CWLTKKKYSSSVHDPNGEYMFMRAVNTAKKSRLTDVTL

SEQ ID NO: 11 - protein, 112 aa, Artificial Sequence
  exemplary CD3 zeta intracellular signalling region
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 1896 aa, Artificial Sequence
  exemplary protein of the invention GD2 ASS+OTC
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAKGSGEGRGSLLTCGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 13 - protein, 1521 aa, Artificial Sequence
  exemplary protein of the invention GD2 ASS1
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 14 - protein, 1463 aa, Artificial Sequence
  exemplary protein of the invention GD2 OTC
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 15 - protein, 1637 aa, Artificial Sequence
  exemplary protein of the invention Mesothelin ASS1+OTC
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPMQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAKGSGEGRGSLLTCGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 16 - protein, 1262 aa, Artificial Sequence
  exemplary protein of the invention Mesothelin ASS
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPMQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 17 - protein, 1204 aa, Artificial Sequence
  exemplary protein of the invention Mesothelin OTC
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPMQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 18 - protein, 1644 aa, Artificial Sequence
  exemplary protein of the invention CD33 ASS+OTC
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAKGSGEGRGSLLTCGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 19 - protein, 1269 aa, Artificial Sequence
  exemplary protein of the invention CD33 ASS1
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 20 - protein, 1211 aa, Artificial Sequence
  exemplary protein of the invention CD33 OTC
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 21 - protein, 1651 aa, Artificial Sequence
  exemplary protein of the invention EGFR ASS1+OTC
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRGVPSRFSSSGTGTDFVFTIENTLSEDVGDYYCLQSFNVPLTFGDGTKLEKALEQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAKGSGEGRGSLLTCGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 22 - protein, 1276 aa, Artificial Sequence
  exemplary protein of the invention EGFR ASS1
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRGVPSRFSSSGTGTDFVFTIENTLSEDVGDYYCLQSFNVPLTFGDGTKLEKALEQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 23 - protein, 1218 aa, Artificial Sequence
  exemplary protein of the invention EGFR OTC
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRGVPSRFSSSGTGTDFVFTIENTLSEDVGDYYCLQSFNVPLTFGDGTKLEKALEQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 24 - DNA, 1236 bp, Artificial Sequence
  sequence encoding ASS1
atgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaaacttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaa

SEQ ID NO: 25 - DNA, 1062 bp, Artificial Sequence
  sequence encoding OTC
atgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggcttagcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattt

SEQ ID NO: 26 - DNA, 789 bp, Artificial Sequence
  sequence encoding GD2 scFv
gatattctgctcacacagaccccactctccctgcccgtgtcactcggggatcaggctagcatttcttgccgctcatctcagtctctggtccaccggaatgggaacacatacctccattggtacctccagaaacctggacagagccctaaactgctcatccacaaagtctcaaatcggttctccggcgtgcccgatcgctttagcggatccggatctgggaccgacttcacactgaaaatctcacgagtggaggctgaggatctcggcgtctacttctgtagtcagagtacccacgtcccacccctcacctttggcgctggaacaaaactggagctgaaacgagccgatgctgctcctaccgtgtccatctttcctggctccgggggaggcgggagcggaggcgaagtgaaactccagcagtctggcccttctctcgtggaacctggcgcttctgtgatgatctcctgtaaggcctctggatcttcctttaccggctacaacatgaactgggtccggcagaacattggcaaatccctggaatggattggcgccatcgatccttactacggcggcacatcatacaatcagaaattcaaggggcgagcaacactcactgtcgacaaatcttcatccaccgcctacatgcacctgaaatctctcacatccgaggatagtgctgtctactactgtgtctctggcatggaatactggggacagggaacttctgtcaccgtgtctagtgccaaaaccacacctccctccgtgtacggacgagtcactgtctcatct

SEQ ID NO: 27 - DNA, 744 bp, Artificial Sequence
  sequence encoding CD33 scFv
ggatccaacatcatgctgacccagagccctagcagcctggccgtgtctgccggcgagaaagtgaccatgagctgcaagagcagccagagcgtgttcttcagcagctcccagaagaactacctagcctggtatcagcagatcccaggccagagccctaagctgctgatctactgggccagcaccagagaaagcggcgtgcccgatagattcaccggaagcggttctggcaccgacttcaccctgacaatcagcagcgtgcagagcgaggacctggccatctactactgccaccagtacctgagcagccggacctttggcggaggcaccaagctggaaatcaagagaggcggcggaggctcaggcggaggcggatctagtggcggaggatctcaggtgcagctgcagcagccaggcgccgaggtcgtgaaacctggcgcctctgtgaagatgtcctgcaaggccagcggctacaccttcaccagctactacatccactggatcaagcagacccctggacagggcctggaatgggtgggagtgatctaccccggcaacgacgacatcagctacaaccagaagttcaagggcaaggccaccctgaccgccgacaagtctagcaccaccgcctacatgcagctgtccagcctgaccagcgaggacagcgccgtgtactactgcgccagagaagtgcggctgcggtacttcgatgtgtggggagccggcaccaccgtgaccgtgtcatct

SEQ ID NO: 28 - DNA, 723 bp, Artificial Sequence
  sequence encoding Mesothelin scFv
atgcaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtgtaggcggttcaggcggcggtggctctggcggtggcggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtggttaccctctcacgttcggtgctgggacaaagttggaaataaaa

SEQ ID NO: 29 - DNA, 768 bp, Artificial Sequence
  sequence encoding EGFRvIII scFv
caggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctctagagcagaaactgatctcggaagaagatctggcgaagccc

SEQ ID NO: 30 - DNA, 17555 bp, Artificial Sequence
  sequence encoding ASL
gacgccatcccggccagaaaagccctggccagtggcgggcgcgacactatccgtgcggccaggcggaggtgagtgcgcggcggccggatgggcgggacgggcgtggaggacgccgagcaccgtggcgcgcgctcacgtccgcgtccccaagggctgcgctccctcaagcgcagtgcccagaactcggagccagcccggcccgggggaccctgctggccaaggaggtcgtcagtccggtcttgtcttccagacccggaggaccgaagcttccggacgacgaggaaccgcccaacatggcctcggaggtgagtgggacctcggggactccggtcctcctagcctccaaaggagagagtgggggcgccagacctgcctcgggccaccctgctgggaatcgccctccaggaagcaattttgaaaattacctaggaagcctgcacccccagccctcccgggcgcatcatctggagcccagcagtcacctttaccaggactcaccagtatccgcaggcagcccttgtggcaaacccaccaacccacactactaggggtagagtggctctgccctcacctcacagtgatgcctgcctggccaggaaaagtggctcccaagccttcagccttccaactcttccttccttcttaccacgtgtcctcctgtcaggtcccaccccacaccacatcccttctcctgctagagcaattgtccctgtttatagaataaagctcagcccctaagtgttcttgtccttgactgtggcatgtggaaagagccaggaaaaaggggacgtcgcctcgtggctccagcaaccctggtgcctggtcccttcctgtctcactggaccctgcctcttagggtcagtggctcctggcctctcctcctgaccactgagatgctggattcccaggcagaggttttccttccttgggccatagttgatttatctggcaatgggggtaataatagctgtcggcctcactctgtaaggcactagattatgaggccattgctttggacccttcaggtgagaggggctgttcgcctgatgcttgatgaagggaactccgggaagcaggaggtctgggttccaggcccttttggccttcattagctagcaattcacttcctctttctcaatgccctgcaagctcagtgccctgcaagcttctgggtcatggcaggggggtagggcctggactttggagccaaacagacttggtttctgtaccagtcacttgagccctttaaacctctttcctcatttgtgaaataggggtaatattgcccacctcataaaaggccgtaataacatatgtgaaatccctagcacagggctgagcaacagtaggtgctcaataaatggtggctaaccacaacaatactgatatttctactttgggaggccgaggcgggaggactgctgaagcccaggaattccagaccagccagggcaatgtaatgagaccctgtctctacaaaaagattttgaaaattacccaggtgtggtggcgtacacctgtagtctcacaggaggcggaggcatgaggattgcttgagcccaggagtttgagactgcagtgagcatgatctcaccactgcactccagcccaggcaacagagtgagatccagtttcaaaaaaataaaaataaaaaaacctggcaggcatggtggctcacacctgtaatcccagcactttgggaggtcgaggtgggcagatcacctgaggtcaggagtttgagaccagcctggccaacatggtgaaaccccgtctctactaaaaatgcaaaaattagctgggtgtagtggtgggtgcctgtagtcccagctattcgggaggctgaggcaggagaatcgcttgaactcgggaggcggaggttgcagtgagctgagatcgcaccactgcactccagcctgggcgacaaagcaagactccacctcaaaaaaagaaaaaaaacccacaagtcccaaaaccaaaactggtatttcccatgtacattcgaccttaactgttgctcattcaacccagcccaactcagtgccccatcccctggccctgaagagaccattctggcccagatgtgtcctggcttggagtagcaccttctgctaccacactaggcctccactctcctcagtgcccaaggggaggcacctcactctgatctccctgtggggtcctcttatgcctgccagtaaggataggagtttggttctagagcagagtggtctggcttccaacaagcccagtgttctaggtgacctcaggttgccccaggccttcctgtaggttggcactaattggtttggctgcagctccacttattaagtagttatttttattactaacaacctaggcagggtggcccaggagcccgctgggggaggcggtgccaggctcctggctgaggggcaggctggccccaattctgactggctggctccagtgatcaggaccagggccccacgtggtgctttgctggagatctaggcttggtggggcagctgtggggtaaggggcaggaccagctcaaagatggggtgggggcggaggctgcctctgcagggcagagtcctttggcagtcgggactgttgggcatagaggagtcagctcacagctcagggccagagcactggagaggtctctggggtgcatacaggaaccaggagtggagctgaagcatgtcctatcccctgccagccctcccttagtaacagctggcatttctcgagtccttcctgagcaccaggcaccgtgttatgtatgcaatttgcaaatattatctgattaaatgctcacaataaggctatcagagaggtactattattatccttatcttattattattattattattatttttaagaaggagtcttgctctgtcgcccaagctggagtacaatggcgccatctctgctcaccgcaacctctacctccaggattcaagtgattctcctgcttcagactcccaagtggctgggactacagcttcctgccaccgcgcctggctaatgtttatatttttagtacagatgaggtttcaccatgttggccaggctgatctcgaactcctgacctcaagcgatccacctgcctgggcctcccaaccctgagggtttttgtttttgtttttgttttgagacagagtcttgctctgtcgcccaggctggagtgcagtggcacgatctcagctcactgcaagctccgcctcccgggttcacgccattctcctgcctcagcctcccaagtagctgggactacaggcgcccaacacccccatgcctggctaatttttttgtgtgtgttttcagtagagacggagtttcaccatgttagccaggatggtctccatctcctgacctcgtgatctgcccacctcagcctcccaaagtgctaggattataggtgtgagccaccacgcccagcccctgaggtttaataataggtgccaggccaggtggttaatagaagtctggggcattgtagggggacagaggaggatatatgtccccattggccattgtagactcccttccacaaaaaggacgtcagtgaagtgacatgcccacctctaccccaccctcctcccagtcctgggcactagggctgctccccaggtgttctgtaccccctccccactctgtcccatgccctggcctctgccctctttcaaaacatagatgtggctggcgcctaggctcatgcctataatctcagcactttgggaagctgaggctggaggacagcttgagcccaggaattcaagaccagcctgggcaacatagtgagaccctgtctctaccaattattttattttattttattttgttcatttatttatttattttgagacagagttttgctctgtcacctaggctggagtgcagtggcgtgatcttggctcactgcaacctccgcctcccgggttcaagcgattctcctgcctcagcctcctaagtagctggaactacaggcgagtgccaccacgcctggctaatttttgtatttttagtagagaccaggtttcaccatgttgaccaggatggtctctgtctcctgacctcatgatccacccaccttagccttccaaaatgctgggattacaggcatgagccaccactcccagtcctataaaattttaaaaaaatgtctgggtgtggtggcgcatgcttgtagtcccaactattggggaggctgaggcaagaggattggttgagaccaggagtttgaggctgcagtgagctatgatggtgccaccgcactccaacctgggtaacaaagtgagaccctgtgtctaaaaaagaatttaaaggccgggtgtggtggctcacacccgtaatcccaggactttgggaggccgaggtgggcagatcacgaggtcagattaagaccatcctggctaacaaggtgaaaccacgtctctactgaaaaaaaaaaatacaaaaaattagccaggcatggtggtgggcacctgtagtcccagctactcaggaggctgaggcaggagaatggcgtgaatctgggaggtggagcttgcagtgagccaagatcctgccactgcactccagcctgggtgacaaagagagactccatctcaaaaaaaaaaaaaaaaaaaaagaattaaaaaagatttttttaatgaacaaaacaggctcggcacagtggctcatgcctgtaatcccaagcacttcgggatgccaaggtcaggggatcacctgagatcaggagttcgagaccagcctgaccaacatggtgaaaccccgtctctactaaaaatataaaactcagccaggtgtggtggcacacgcctgaaattccagctactcgggaggctgaggcaggaaaattggcttgaagccgggaggtggaggttgcagtgagccgagatcacgccactgcactccagcctaggcaacagagtgagactctatctcaaagaaaacgaacaaaacatagatgcctacataccattcctctgcccttggctcctggggagtaagggatcacccagtgacctcctagaaggccagtgacaatggggggtgtcagggtgcttttcagagccaagggagtggtaggaattgggatcttagtccagctccaagctgtgagggagagagttgcagggcacttaagcttggtggagaccctcaaggcctctttgcctgtccctgcagcaaaggttctggacaccagagccaagtccagaagccctggtggaacaggggtgaaaagcataggttctgacttcagactgctgggccgcagccctggctatcccaccccaggtgagagcaggctgctctgtgcctcagtttccccatcttcatagtggaattgtattggtgcctacccagagggttgtgtcaacaattaggatggcacctagcaccttggtcagtggtgggaaaggttccagaagttctgctgtggtcccaggggtgtctcaggccctgccatcatctccttggaggggtgccatgtggtgggaaagaaccccaacttcaaggccacacacagtggctcatgcctgtaatcctagcactttcagaggccaagatgggaggatcacctgaggtcaggagttcaagaccagcctggccaacatggtgaaaccccatctctactaatgatacaaaaattagctgggtgtggtggcacgtgcctgtaatcccagctacttgggagtctgaggcaggagaatctcttgaacctgggaggcagagtttgcagtgagctgagatggcaccactgtactccagcctggccgacaaagtgagactctgtctcaaaaaaaaaaaaaaagaacccaaacttttggtgttcagccatgttcccatgctcactcccagggtggtgactctgggaaggtctcagcctccttgtctgcccagttagaatgatctgatgcccctgctaccatcagacttgataagtttcccaaagactctttgcaagaagcactgttctggagggtggaggagagactaattgttcttgctctcctggccagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtacttgccgtggcccaagccccacccaaggccccttccctgtggccccaggctcccaccaaatccctgagcaaacagtgcagtgttgcccatctgtggtttcacattgaactaattatatactcaagtgctgtttaactgtgtgccttgatgactgcctctctccatcctttaatgacccctgtggcccacatggctcatgggtaaaggtgtgctgggcctgagatgccccctcccagggtgcgcttccaggactcagctcctgggcagggacagtcagtcaccagggatagggtgggaccaaggcaggggctctcttggctgctgatgcctgctcacctgaccccggcattgctgctacccactacaggtggctgaggagtgggcccagggcaccttcaaactgaactccaatgatgaggacatccacacagccaatgagcgccgcctgaaggtacgacccctggagccccaccgctttccttgcctcccctctccaccttgcccagggccactttgagcattagcaccattctgtttacttcgccattggcagacagcatgtgagacctcaggacatgagccaggcaccctggctcatgcctataatcctagcactttgggaggctgaggtgggaggattgcttgagaccaggagttcgagaccagcctgggcaacataatgaggtcccacagctacaaaaattaaaaaaagaaaagaaaaaaagaacaggcctcagcagaaatggcgagagatttggggaggacccggagccctggggtatggaggtaggttggcagggctgatgaggaaaactgccctgcctgggttgactcctctgggggtatagaccgtgaccctgggtctcccttcacctccaggagctcattggtgcaacggcagggaagctgcacacgggacggagccggaatgaccaggtgctttagcccctccaccccctgctccgtgttgtcccaaccttgaggagcccagggggcagttagagttctgcagcggtcctggctcctcagggaagcaacacatcggcctccctgagcaccatctcctccttgcacaggtggtcacagacctcaggctgtggatgcggcagacctgctccacgctctcgggcctcctctgggagctcattaggaccatggtggatcgggcagaggcgtgagtcctacagggacacccagggggcagacagaggtgtgatggaagcctgaacaggagacctagggggcaggggtgaacagcgtgggggtgccaggccctgggggacaggggcatcccagaactccaggatcgaggcagagcagccaggagtgggccatttcctgcaggccccaatactcccatgccagtctagctcagcaggcagagaagactaacccttcgtggggctgggtgcggtggctcacgcctgtaatctcagcactttgggaggccgaggtgggtggatcacctgaggtcaggagttcgagaccagcctggccaacatgggaaaactctgtctctactaaaaatacaaaaattaggcaatgtggtggtgtgcgcctgtaatcccagctactcgggagcctgaggcagaagaactgcttgaacccgggaggaggaggttgcaatgagccgagatcgcgccattgcactccagcctgggctacagagcgagactcctgtctcaaaaaaaaagaaaaaaaaaaaagaaaactcaccatttgcagatttgaaggcaggaagctaagccaagcacagctagcttggctgtgcctggagcagccagagtcactccccacactgcctgtcccccagatcccccatcctaagcttcgcctccccatccagcccatctggcaaaagacagagccaaaggctgcctcctgctggcctcatttcaggctttggcttctgggacctggtgtctttgggactggatttgttccttgcagacctggacgaagagctgctgagaagtctccatgtgttgtcagagacccctcctcttcctcaactccctgtgacccctgttgtgcagacttgggggaaaacaagggcacaagaattgtcacccagcaggtggtgtggggctgctaggaggaacagggagtgtctgctactgagttcagggtttctttaattttttgttgttgttgtttgttgttgtttttttttttttgagacagggttttgctctgtcacctagtctggagtgtagtggcgctatctgagctaactgcaaactctgcctcctgggttcaagtgattctagtgcctcagcctcccaagtagctgagattacaggtgtgcaccaccatgtccagctaatttttgtatttttttcagtagagatgggttttgccatgttgaccaggctggtcttgaactcctgagctcaggtgatctgcccgcgtcggcctgccaaagtgctaggattacacccataagccactgcgctcagcttaatttttaaatttttaactttttaaattgtctttagagatgagatcctgctctgtcacctaggctggagtgcaatggcttggtaatagctcactgcagtctcaaactcctggactcaaatgatcctcccacctcagctttctgagtagctaggaccacaggtgtgcaccacctgtgagacagagtcttgctctgttgcccaggctggagtgcagtggcgtgatctccactcactgcaacctctgcctcccaggttcacgccattctcctgcctcagcctcccgagtagctgggagtacaggtgcccaccaccacgcccggctaattttttgtatttttagtagagacggggtttcaccatgttagccaggatggtctcaatctcccgacctcatgatccacccacctcggactctcaaagtgctgggattacaggtgtgagccaccgtgcccagccgcgaattctttaaattttttgtagaaacagggtctcactatgtggctcaggctggtctcaaactcccggccttaagtgatccttccctcttggcctcccaaagtgctgggattaaagacttgagccaccgtgcctggccttgagtacagaatttcttcatggggtgatgaaaatgttctaaaattggttgtggtgatggttgtacagtaaagtgtaaactttaaatgagtaaattgtgaatgatatctcagtaaagctggtttatttaaaacaacaggccaggtgctgtggctcacgcctgtaatcccagcactttggaaggctgaggcgggtgaatcacctgaggtcaagagttcgagaccagcctggccaacatggtgaaaccccatctctactaaaaatacacaaaattagctgggtgtgatggtgggcacctgtagtcccagctacttgggaggcaggagaatctcttggacctgggaggtggaggttgtagtgagccgagatcacgccactgcattccagcctgggcaacaagagcgaaactctttctcaaaaacaacaacaacaaaaaaacaggccaggtatggtggctcatatctgtaatcccagccctttgggaggccaaggcaggaggactgcctgaaaccaggagtttcagaccactctgggcaacatagcaagaccccatctttttttttttttttgagacggagtctcgctctgtcgcccaggctgaagtgcaatggtgcaatctcaactcactgcaagctctgcctcctgggttcatgccattctcctgcctcagccctcctgagtagctggaactacaggcgcccaccactacgcccggctaattttttgtatttttagtatagatggggtttcaccgtgttagccaggatggtctcgatctcctgaccttgtgatctgcccgcctcggcctcccaaagtgctgggattacaggcgtgagttaccgcgcctggccacaagaccccatctttacaaaaaactaaaaattagctgggcatggtggcatgtccctttagtcccagctactcaggaggctgagacaggaggatcgcttgagcccaggagatcgaggcagcagtgagctatgatcatgccactgcactccagtctgggcaacagaacgagaccttgtctctaaaaataaaaacaaaacaaaacaacaagaaaacaggaccatcactcacagcacctctgcctctgccctgcctacttgaatgaggtgcagggcatctcacctgctcagagcagcccttgaatgagccccagctatttctagggtcctcaaacgaaacctcccacggccaagtcatacccaacatgggcctcctcccctattctggcccctgctcggagatgctgagtgacagaggctggacttggggtgtttctggcaaagcctcactgcaggaagccccacagctcaggcccagtccttggttcacacggtcccacttccagcttcttttgcccttaagactgatttgtccctgggagatcaccagatccctcattcaggtggagtgctgcagcgtgacactttttccaggggtgacccaggcctgcagggttccagtgtcacaggcaggccttgcatgagcctccacccgagcttctgctcctcctctcccacagggaacgtgatgttctcttcccggggtacacccatttgcagagggcccagcccatccgctggagccactggattctgaggtgagccaggtgaggtgcaggggctgtgctagaggggaggaccccggctgccctgaccctcctgcccctggcttcccacagccacgccgtggcactgacccgagactctgagcggctgctggaggtgcggaagcggatcaatgtcctgcccctggggaggtgggtgaggctccagtgccccgagggcctggtgggggtggctgctgcatagccttagggattgacagagctgggaagtgcagagtgggacagaaaaccgccttatctgctcagcgggggactctgcatggagccccagctctcgctaaggtgacgaccaagccattgaatgtgtctgagcagggccagagccctccagcaaggctcctggcaagcccagcctgctgccctcagcctgacatgtgggaacatgtgtcaggagacaagtgtcctgcacccagggtgacttagtgcttggggacaagtgttttgtggacacttggggacaagtattctgtacccaaggagactgggccagggaagaggctaagcgccaggtggttgccctggcaaccaggacttggttctctgtgtgtgcgttcgtgtgtgtgtgtgtgtgtgtgtgtgtgtgtgtgtgtgtgtcagggctgcctgccaggagccctggtcaccatgaatccctgtccctgcagtggggccattgcaggcaatcccctgggtgtggaccgagagctgctccgagcaggtgagacgtcctgcccctcctccccagggagaatcaccctcagcacccgccaagacctgcagacacacctgaaaccagagggcaggggcctgtggctcctggtgaaaccttcattcattgcctatgggcactgaggtcatcaagttcaggggtcactcatggcagggatgcctggtactgagagactcagggctcctgcctccctcctgggactgtgcaaaagatccctccccccagctgttgccccaccctgatcaggggagggggctgggcaacctagttgggggagagggggccactccctgtcctccagcttagccctgcttcctcccacccccccagaactcaactttggggccatcactctcaacagcatggatgccactagtgagcgggactttgtgggtgagtcctggggagccagtcccctgccctgtgcctcactttagtccttcagcccagcttctctccagtttcctcccacacctccacggacaggctggttgtggtgatattgtacactgaagtataaaccttaaatgggtaaagtgggtggggcatggtggttcaccatgcccagcactggccaacatggtgaaaccccatctctactaagaatacaaaatttagctgggtgtgtggtggcaggtgcctgtaatcccagctactcaggagttctgaggccagagaatcacttgaacccaggaggcggaggctgcagtgagccaagatcacgccagtgcactccagcctgggcaacaagagcgaaactccatctcaaaaaataaaataaaataaaataaaaataaataggccaggcatggtggctcacgcccgtaatcctagcactttgggagtccgaggcaggtggatcacatgaggtcaggagtttaagaccagcctggccaacatggtgaaaccccatctctactaaaagcacaaaaattagctgggcatggtggtgcatgcctgtaatcccagctactcgggaggctaaggaaggagattcgctggaacctgggaggtagaggttgcagtgagccaagattgtgccactgtactccagcctgtgcattgggagcgagactccatctcaataaataaataaataaataaatggataaattgtatgtgagtgataactcagtaaagctggtttatttaaaacaacaacaataacaaaaaacacgctaggtgcaatggcttacgtttgtaatcctagcactttgggaggccaaagcagaaggattgcttgagcccacaagtttcagaacagcttgggcgacatagcacgaccccatctttgcgaaaaatgaaaatttagccgggtccccccaccgcctaacctcctcctgccccctgtatggtcaggctgggtggggatgggagaggcctggtgactgggaaccttttctcccagccgagttcctgttctgggcttcgctgtgcatgacccatctcagcaggatggccgaggacctcatcctctactgcaccaaggaattcagcttcgtgcagctctcagatgcctacaggtaagccctgaactgccacctccatctgccgctgccggcctctgtatcccccgccgcccgcggacgtggctgccttcctccccgtcccacccctccgccagacctggccattgcggcgctggaccagccaagggtccagccccttcagcgccagcacctctgtccccagcacgggaagcagcctgatgccccagaagaaaaaccccgacagtttggagctgatccggagcaaggctgggcgtgtgtttgggcgggtgagcaaggcagggggaggggcggggcctctgggctgatggtgggtggccaggggggcaggatcccgggtccagcccctgtgcctccctcttcccgcagtgtgccgggctcctgatgaccctcaagggacttcccagcacctacaacaaagacttacaggtgcgaggccgggggaggcctggctagtacgtgccagttctcagggctctggcacactcaggcagggccccaccccgggattgccatacatcctcccatcctgtgcacacagctccatccgtggctgcccttgaactctctgcccttcctttgttggggtattgagtgttcttcccatggaaggcagtggggatgcctcagtgggggggtggggctgtggggaccctgggtgccagggggctgctaggccctcacctcctgccatgtgcctcccaggaggacaaggaagctgtgtttgaagtgtcagacactatgagtgccgtgctccaggtggccactggcgtcatctctacgctgcaggcaagacatcacccccctgcttctcctcccctaggtcccaggcactggggtgggcatgcggggagggtggccttgggaggaggtgaggtggggctggaggacctggggcagggaaggagaggtgtgctcgctcctgctcctggggaacagggaaaggacagaaactgctgccatgcagtggaagtagatgagactcagggggcctggggcctgtcaaatggcctgaccagaactctttaaaaaaagaaaatctaaacaaaaggccaggtgcagtggctcatgcctggaatctcacactttgggaggccgaggcagatggagcacttgaggtcaggggtttgagaccagcctggccaacatggcgtaaccacgtctctactaaaaatacaaaaattagccaggcgtgatggcccacacctgtaatcccagctactcaggaggcggaggcagaagaatagcttgaacccaggagatggaagttgtagtgagccaagatcatgccgctgcactccagcctggaccacagagtaaaactccatctacaaatatataaattaaattaaattaaattaaatatctttaaaaaacattttttagagacagggtcactctctgtcgcccaggctggagtgcagtggtgcggtcgtagctcactgcagcctcaaactcttgggctcaagtgatcttcccacctcagtctccagagtagctgggactacaaacatgcgccaccacgcctggctaatttttttattttttgtagagacagggtctccctatgtttcccaggctggtctcaaattcctggccccaagccatcctcccaccttggtctcccaaggtgctgggattataggcatgagccactttgcctggctgatttcttttaaaatcaattattatgggaaatttatgtatataacagctagagaatgcataatgaaccctatgtaccgacacccagcttcaatgataatcaactcacggacatcctggctccagctgtctttacccacagctctctcccactcccttacccccttattttgaagcaaattcccatcatcacatcatttcattcctaaatagttcaggatatgtcttgaaatcagtgtttcttggctgggtgcagagcctcatgcctgtaatcccatcaatttgcgagactaaggtgggcagatcactcgaggtcaggagttcgagaccagcctggccaacatggcgaaaccccgtctttactaaaaatataaaaattagctgggtgtggtggtacacgcctgtaatcccagctactcaggaggctgaggcaggagaattgcttgaacccgggagatggagactgcagtgagcagagatcacgccactgcgctccagcctgtgtgacagtgcaagactccatctcaaaaaaaaaaaaaaaaaaaaaaggcagtgtttctggaggctagtcccccaactagcagcaccagcatcacctcagaagtcctgagaaatgtgatgtgaggccccactccagatggctgaatcagagactctgggggtgctccccagcaatttgtatttttcttagtaaattctccagtggctaggcctggtggctcatgcttgtaatcccagcactttgagaagctgaggcaggagaagcgcttgagcccaggagttcaaaaccagcctgagcaacatagcgagaccttgtctgtaaaattaaaaaaattaaattagccagtcgtgatggcgtgtacctgtggtcccagccacttaggagactgatgtgggaggatcccttgagcccaggagctcaaggatgcagagagccaggattgtgccattgcactccaacatgggcgaccctgtctcaaaaaagcccaaaacaacaacaacaaattagctaggcacggtggtgtgcatggctgtagtcccagctacttgggaggctgaggctggaagatcccttgagtccaggctgcagagggctataatggccactgcactccagcctggacaacagagcaagaccctgtctcctaaaacagaaaacaaatcctccaggaacatctgatgcatgctgaagataaggactctttgaaaacataaaggccagtaaaacatacaggccagtaagtgttcatagcacatgtaaatattatcgataattatgagaagatggttcaagttgagagtgagacagagccgagtgggtaagagagtatctgcccaaggcagggatgtcctggcagaggggcaggtcctgggcctggcagcttcagatcccagggtccccagggctcaccactcgcccacctgtgcccccagattcaccaagagaacatgggacaggctctcagccccgacatgctggccactgaccttgcctattacctggtccgcaaaggggtaagtgtgtagcagccagggggagggtgaggagatggggtgccccccccagagggtgggggagctcaggaatgggtgcaagcggcccagcctggtggctcacccctgtaatcccagcactttgggaagccgaggtgggcgggtcacttgaggccaggagttcgagaccagcctggtcaacatggtgaaaccccgtctcttttgatgtaaaaatacaacaattagctgggtgtggtggcacactcctgtaatcccagttactcgggaggctgaggcaggagaattgattgaactgggaggtggagtttgcggtgaggtgagatcgcgccactgcactccagcctgggcaacagagcgagactttgtgtcaaaaagaaaaaaaaaaaaaaaaggaagggggtgcaggcaatggaggcagatcagggcatggagaaacctgcctcagcgccatcttcctccctggcacccagatgccattccgccaggcccacgaggcctccgggaaagctgtgttcatggccgagaccaagggggtcgccctcaaccagctgtcactgcaggagctgcagaccatcaggtacggcccatccccttccccatgctgcctcctaggaagtgagcctgggtgcctggagcccagggtggcctggcgccctggcccacctcttcctctctccccagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcctaggtcctcccacacctgccccctaataaagtgggcgcgagaggaggctgctgtgtgtttcctgccccagcctggctccctcgttgctgggctttcggggctggccagtggggacagtcagggactggagaggcagggcagggtggcctgtaatcccagcactttggaagggcaaggtgcgaggatgcttgaggccaggagtttgacacagcctgggcaacacagggagacccccatctctactcaataataaaacaaatagcctggcgtggtggcccatgcatatagtcccagctacttgtaaggctgaggtgagaggacacttgtgcccaggagtggaggctgcagtgagctatgatcacgccactgcattccagcctggataacagagtgagaacctatctctaaaaataaataaataaacgaaaaataaa

SEQ ID NO: 31 - DNA, 8184 bp, Artificial Sequence
  sequence encoding ODC1
gaagccgggggcgggggccacgcgtggggcaggcggtgctcggctcggctgacgtcggcccgccggcgccccaccagctccgcgcgggcccgggttggccaccgccgggcccccgcccctcccccggcggtgtcccggccggaaccgatcgtggctggtttgagctggtgcgtctccatggcgacccgccggtgctataagtagggagcggcgtgccgtggggctttgtcagtccctcctgtagccgccgccgccgccgcccgccgcccctctgccagcagctccggcgccacctcgggccggcgtctccggcgggcgggagccaggcgctgacgggcgcggcgggggcggccgagcgctcctgcggctgcgactcaggctccggcgtctgcgcttccccatggggctggcctgcggcgcctgggcgctctgaggtgagggactccccggccgcggaggaagggagggagcgagggcgggagccggggcgggctgcgggccccgggccccgggcacgtgtgcggcgcgcctcgccggcctgcggagacacgtggtcgccgagcgggccacgaccttgaggcgccgcttcctcccggcccggggttctcccgcggctggataagggtgatccgggcgcctcgttctgcccccgtcttcacagctcggggctggaggggcctaggggagacccacccggagaccctgcggccccgcgccggcctctttcccaacccttcggcggccgcgcgctggccggggagccgttggggaggccctggcggccgcgcagcaggtgcaggggcgcagagcccgggctcgccttggtacagacgagcgggccccggccttggcgccttcagtttccttccagtttttattttcgctgtgtctacagagcagatgacaccaatttggaaacccgcgagagtgggtagagctaagatagtcttgctgtagtagctgtgatattagatgctcggccatgacttagaggtgtttatttaaggactgtgaatgactcggtgatttcggaaaagcttggcttagatgaacggacatacacaggggagacagccctaaggtttgcagaaaaggctgattgtgctgtttgcgaagtcgaaataattggtgaaagtgtagaaggcagaacctctcaggaatgtctggggaggacaaagaatgtgttggctgactttgtttaaacataaaattgggcagactttaattgatttgtgaaatttttttcaaagtttgtttgaattagcccctatctcttctaacattatcctcttgtgctaattgattgaccattttaaataacttagctgttacagaaagaccgaaaggtgttcttcagtaaaatatattcaagtaagttacttaagtaacgccttaaaagatacagaaaagcaaaaaagtattggcgtattaaaaagaaatcaaaactttccaagtttaggcctgaacattgccttaaaaatatttaataaggcctcaaatgacccagtccgagactgcatgagcctatttattattaaattgtaaatattcttcatataaacaaaaatatataaccatgtctgtaacaaaaatggttttgctagcgttgttactctcttcccttctccgaggggtgatttaggcaacttcggaggttgacaatgccaagcagtcacaatagatagagctttaaagcaaattctatgcatgggtttggatttatgacaggcccgtcaccctgggcctgtcatagtaccccatgccagagcaaactgtgtccccgaaccattgcctggcctctgtgcccgtaggctgctggcactgaagtgggttgcacagtggaaaagaagaaagctctacctggcagaaatttttaaaggttaaaataaataattttaagaaagctggttcacaaggtgccacatttgatgaaagcaaaatacagtggcttttattgttactagagtgatgttcttgcttgtttttcttttttggtgaagttagccccaaattattctcatagctaagcaaatacgagagtgactgtaaggacagttggcattcccggaattgctaaacttggtaggcaacgctggtttaagaatactgagttctagccgggcgtggtggctcacgcctgtaatcccaacactttgggaggctgaggcaggcggatcacctgaggtcgggagttggagaccagcctgactaacatggagaaacgccatctccactaaaaatataaaattagccaggccccgggtgtggtggcacatgccggtaatcccagctactcgggagactgaggcaggagaatcgcttgaacccaggaggcggaggttgaggtgagccgagatcatgccattgcactccagcctgggcaacaagagtaaaactctgtctcaaaaaaaaaaaaaaaaaatactgaattctgatcaggtaacagcaactgtaatacaatgtgataagttgacttgaagattacagtttttaagaagtatatacccagctaatacatgaaaattaactcgtaaaatctcaaatgctccagacatttccatgatgcctgttggtcagtaaaaatcattctaagacttagtggaagtaggaaatgtttgtatggctgtgtataaaggctataatgtaatcccagcactttggaagaccgaggcgggtggatcacctggggtcaggagtttgagacccacctggacaacgtggtgaaatcctgtctctactaaaaacacaaaaattagccgggcatggtggcaggcgcctgtaatcccagctgctggggaggctgaggcaggagaatcgcttgaacccgggaggcagaggttgcagtgagccaagattgcaccgctgcactccagcctgggtgacagcgtgagactctgtctcaaaaaaaataaaaaagtctataatgctattttaagtttctaaggaactgaaactgctctgaaataaatcagaccattataagacttttttccatatcagtgagctaagtgcagataagcttctgaaacttgcatgctagatttttttggtacaaatatttgaaatgcttagtgtgctgccttggaaaaacctggtattttttgttgtgtccttatactgccaaggtttatggaatcatgtaccttatgcctagtaataattaggatgaccaggccagtgagtggttcatatccggggcatgattagctctgcgtgtgctcagccagtgccccatcttcaactcgatgtgttcctaaggtagacagcaaattccctattttatttctcagattgtcactgctgttccaagggcacacgcagagggatttggaattcctggagagttgcctttgtgagaagctggaaatatttctttcaattccatctcttagttttccatgtaagtattcagtttacatttatgttgcaggttaatcttaagaattgtattgctaaggcttctaagtgaatttctccactctatttgcattttgttgcatttcagaggaacatcaagaaatcatgaacaactttggtaatgaagagtttgactgccacttcctcgatgaaggttttactgccaaggacattctggaccagaaaattaatgaagtttcttcttctgtaagtatatgaggcccatgctggcagtgcagctgagagtgccaggcaagtggaaaactttggcaaggtctaaggaagagcaatgaggcttacatgtcttgttatggaatgtagaaattaattcactggtggtaaattaatagtgataatggtgatactcatatcagtggctagactcaaaagagcaggattcattgtgactgatgggaatgaaggtcgctggctattggtgtggtgtgtggtgaggctgctagtgagtcacctgtgaccactcttgtttcaggatgataaggatgccttctatgtggcagacctgggagacattctaaagaaacatctgaggtggttaaaagctctccctcgtgtcacccccttttatgcagtcaaatgtaatgatagcaaagccatcgtgaagacccttgctgctaccgggacaggatttgactgtgctagcaaggtaagcgatagcagcaggcctcaaaagcgttgtataaaatgggcctggtattccccacgaggcagatacaagttgtgttttttgggcaataaatgctcactaaaggcaaatggggcgggggggtacatgacaacttcccatgcttttctgtttattccacgtgttaagccacatatggatagcatgacaccactcttctttttcagactgaaatacagttggtgcagagtctgggggtgcctccagagaggattatctatgcaaatccttgtaaacaagtatctcaaattaagtatgctgctaataatggagtccagatgatgacttttgatagtgaagttgagttgatgaaagttgccagagcacatcccaaagcaaagtgagttattcccccatctgagggcaagatcgggagcataagatatgtggattcttatcaaacaaacttaaatttctgattattatatttctatactttagtagaaagtagttgaaacccccattgagtcatgaagcctgggactcaaactacagaatatatcagcgacagtatttagaacaggattgtttttattttaattgtggctataagtgaacatctatcatgagacatttgctgcactttccttgcttgtaggttggttttgcggattgccactgatgattccaaagcagtctgtcgtctcagtgtgaaattcggtgccacgctcagaaccagcaggctccttttggaacgggcgaaagagctaaatatcgatgttgttggtgtcaggtgagattttggtgggatagctagaggtcaagacattgaacagtttgagttttacaggctttctcctagtgtttgctattattttaagaaatactaagacacagtgtctcgtctctttattttaccccagcttccatgtaggaagcggctgtaccgatcctgagaccttcgtgcaggcaatctctgatgcccgctgtgtttttgacatgggggtgagtatacgtgaccctgttagggaagggcgggacacaactgacaataactagtcttaattctagagttaactttttatggcagttggttctgtattacatgggtttcagcctatctgctgcatacatttttgttattagctgtggatctggctgacttattttcttgattctaggctgaggttggtttcagcatgtatctgcttgatattggcggtggctttcctggatctgaggatgtgaaacttaaatttgaagaggtaatttagaacaaaactgtaatactcagtagccgttctaataaattcctttttggaatatttcaaaatttaagtgtcttaactaataccacaatgggctgaagtgtcttggtgtgatattttgagtgatttctttgtgctgtctgacattacacttgataccatttggttttctaaagtgtgaatcagctttcccagaagtcttggataattggttacattggaaatcatggctcacacctgtaatccagcacttggggaggccaaggtggtaggatcacttgagcccaggagtttgagaccagcctgggcaacacagtgagaccccatctctacaaaaaaaattttaaaattagcctggtgtggtggcgggcacctgtaatcccagctacttggaaggctgaggtgggaggatcacttgagcccaggaggttgaggctgcagtgagccatgatcatgccactgcactcagcctgggctacagagtgagaccctgtctcaaaaaaaaaaaagaaaaagcatgttgctgtgggcttcctagagaatatgctgactgtagcacatcatcaccccaaatgtgctttgctagacctatgcttcctctccttaaaatacttgaaatgtttagtcacttaggaagttaagccattatattggtgcttgaatttataaaatatatccacatggtttgttaaaatcatgacgtaggcagaataggatttttatcctgttggcatgtatttgttaaaatgttttgacatcttgatgccttcctaggtagtagttagttgcgtactgttctttgataaaaatcatacccataacatcctaaaggagatagggtgcctggaggggaatgaaaacgagccacctgggatatgtagcctggttttcagggagatgttgatgtttttttgcttttgttactttaatgataaacctgtctgttgatgcctggtctcatgatgtcatgtcacaaggccctgtgatgttactcccccatgtgaatttcccacaatgaaggctgctctttcttttctgtttcactctcttagatcaccggcgtaatcaacccagcgttggacaaatactttccgtcagactctggagtgagaatcatagctgagcccggcagatactatgttgcatcagctttcacgcttgcagttaatatcattgccaagaaaattgtattaaaggaacagacgggctctgatggtatgtataaaggacgaatcacttcatgtataactgaaagctgatgcaaaaagtcattaagattgttgatctgcctttctagacgaagatgagtcgagtgagcagacctttatgtattatgtgaatgatggcgtctatggatcatttaattgcatactctatgaccacgcacatgtaaagccccttctgcaaaaggtaatttctgagcatactgtataaaacaattaagaggactggtcacaacacgtgtaattaagtagtacttcctctctccgtctctttatatagagacctaaaccagatgagaagtattattcatccagcatatggggaccaacatgtgatggcctcgatcggattgttgagcgctgtgacctgcctgaaatgcatgtgggtgattggatgctctttgaaaacatgggcgcttacactgttgctgctgcctctacgttcaatggcttccagaggccgacgatctactatgtgatgtcagggcctgcgtggtaagtaagccatgcatgttgatggtgctgccaagaataggcaccttcttggatgtgtgcttcttgtctagacgaataagaaattgtcttgcctaagattaaatatatatggatatttttcctaagaaaagttttagaaaagactgatgagtgtatttctatgtaattggaatatatttaagttcatgccatgtgtcttgtggtttccttattaccaaaacggtgactgaagaaacgcttgctttagaaatacattgaattggccaggtgtgctggctcacacctgaaatcacaacacattgggaggccaaggcagaaggatcacttgagcccaggagttcgagcctgggcaacatagtgagaccctgtctctacaaaaaattaaaaaattagttggccatggtagtgggcgcctgtagtcccagctgcttggctaaggtgagaggtttgcttgagcctgggaggttgaggctgcggtgagctatgatagcaccattgtattccagcctgagtaacagagaaagaccctgtctcagaaaaaaaaaaaatacattgaattgtttcctgatgggaagtaaatactctcatgcccagttaggagtgagtcagggtttttaatatgccactttttctttctcaggcaactcatgcagcaattccagaaccccgacttcccacccgaagtagaggaacaggatgccagcaccctgcctgtgtcttgtgcctgggagagtgggatgaaacgccacagagcagcctgtgcttcggctagtattaatgtgtagatagcactctggtagctgttaactgcaagtttagcttgaattaagggatttggggggaccatgtaacttaattactgctagttttgaaatgtctttgtaagagtagggtcgccatgatgcagccatatggaagactaggatatgggtcacacttatctgtgttcctatggaaactatttgaatatttgttttatatggatttttattcactcttcagacacgctactcaagagtgcccctcagctgctgaacaagcatttgtagcttgtacaatggcagaatgggccaaaagcttagtgttgtgacctgtttttaaaataaagtatcttgaaataattaggcattgggacgtt

SEQ ID NO: 32 - DNA, 1344 bp, Artificial Sequence
  sequence encoding ArgG
atgacgacgattctcaagcatctcccggtaggtcaacgtattggtatcgctttttctggcggtctggacaccagtgccgcactgctgtggatgcgacaaaagggagcggttccttatgcatatactgcaaacctgggccagccagacgaagaggattatgatgcgatccctcgtcgtgccatggaatacggcgcggagaacgcacgtctgatcgactgccgcaaacaactggtggccgaaggtattgccgctattcagtgtggcgcatttcataacaccaccggcggcctgacctatttcaacacgacgccgctgggccgcgccgtgactggtaccatgctggttgctgcgatgaaagaagatggcgtgaatatctggggtgacggtagcacctacaaaggaaacgatatcgaacgtttctatcgttatggtctgctgaccaatgctgaactgcagatttacaaaccgtggcttgatactgactttattgatgaactgggcggccgtcatgagatgtctgaatttatgattgcctgcggtttcgactacaaaatgtctgtcgaaaaagcctactccacagactccaacatgcttggtgcaacgcatgaagcgaaggatctggaatacctcaactccagcgtcaaaatcgtcaacccgattatgggcgtgaaattctgggatgagagcgtgaagatcccggcagaagaagtcacagtacgctttgaacaaggtcatccggtggcgctgaacggtaaaacctttagcgacgacgtagaaatgatgctggaagctaaccgcatcggcggtcgtcacggcctgggcatgagcgaccagattgaaaaccgtatcatcgaagcgaaaagccgtggtatttacgaagctccggggatggcactgctgcacattgcgtatgaacgcctgttgaccggtattcacaacgaagacaccattgagcagtatcacgcgcatggtcgtcagttgggccgtctgctgtaccaggggcgttggtttgactcccaggcgctgatgctgcgtgactctctgcaacgctgggttgccagccagatcactggtgaagttaccctggagctgcgccgtgggaacgattattcaatcctgaataccgtctcagagaacctgacctacaagccagagcgtctgacgatggaaaaaggcgactcggtgttctcgccagatgatcgtattggtcaattgaccatgcgtaacctggatatcactgatacccgcgagaaacttttcggttatgccaaaactggcctgctttcctcctctgccgcttcaggcgtgccgcaggtggagaatctggaaaacaaaggccagtaa

SEQ ID NO: 33 - DNA, 1374 bp, Artificial Sequence
  sequence encoding ArgH
atggcactttggggcgggcgttttacccaggcagcagatcaacggttcaaacaattcaacgactcactgcgctttgattaccgtctggcggagcaggatattgttggctctgtggcctggtccaaagccctggtcacggtaggcgtgttaaccgcagaagagcaggcgcaactggaagaggcgctgaacgtgttgctggaagatgttcgcgccaggccacaacaaatccttgaaagcgacgccgaagatatccatagctgggtggaaggcaaactgatcgacaaagtgggccagttaggcaaaaagctgcataccgggcgtagccgtaatgatcaggtagcgactgacctgaaactgtggtgcaaagataccgttagcgagttactgacggctaaccggcagctgcaatcggcgctggtggaaaccgcacaaaacaatcaggacgcggtaatgccaggttacactcacctgcaacgcgcccagccggtgacgttcgcgcactggtgcctggcctatgttgagatgctggcgcgtgatgaaagccgtttgcaggatgcgcttaagcgtctggatgtcagcccgctaggctgtggcgcgctggcgggaacggcctatgaaatcgaccgtgaacagttagcaggctggctgggctttgcttcggcgacccgtaacagtctcgacagcgtttctgaccgtgaccatgtgttggaactgctttctgctgccgctatcggcatggtgcatctgtcgcgttttgctgaagatctgattttctttaacaccggcgaagcggggtttgtggagctttctgaccgcgtgacttccggttcatcattaatgccgcagaagaaaaacccggatgcgctggagctgattcgcggtaaatgcggccgggtgcagggggcgttaaccggcatgatgatgacgctgaaaggtttgccgctggcttacaacaaagatatgcaggaagacaaagaaggtctgttcgacgcgctcgatacctggctggactgcctgcatatggcggcgctggtgctggacggcattcaggtgaaacgtccacgttgccaggaagcggctcagcagggttacgccaacgccaccgaactggcggattatctggtggcgaaaggcgtaccgttccgcgaggcgcaccatattgttggtgaagcggtggtggaagccattcgtcagggcaaaccgctggaagatctgccgctcagtgagttgcagaaattcagtcaggtgattgacgaagatgtctatccgattctgtcgctgcaatcgtgcctcgacaagcgtgcggcaaaaggcggcgtctcaccgcagcaggtggcgcaggcgattgcttttgcgcaggctcggttagggtaa

SEQ ID NO: 34 - DNA, 1005 bp, Artificial Sequence
  sequence encoding ArgF
atgtccgatttatacaaaaaacactttctgaaactgctcgactttacccctgcacagttcacttctctgctgacccttgccgcacagctcaaagccgataaaaaaaatggcaaggaagtacagaagcttaccggtaaaaacatcgcgctcatcttcgaaaaagactcgactcgtacccgttgctctttcgaagttgccgcatttgaccagggcgcgcgcgttacctatttagggccgagcggcagccagattgggcataaagagtcaattaaggacaccgcgcgggttctcgggcggatgtatgacggcattcagtatcgcggtcacggccaggaagtggtcgaaacgctggcgcagtatgcgggcgtgccggtgtggaacgggctgaccaacgagttccacccgacccagctgctggcggacctgatgaccatgcaggagcacctgccgggcaaggcgtttaacgagatgacgctggtctacgcgggcgatgcgcgcaacaacatgggcaactcgatgctggaagcggcggcgctgaccgggctggatctgcgcctgttggccccgaaagcctgctggccggaagagagcctggtggcggagtgcagcgcgctggcggagaagcacggcgggaaaattactctgacggaagacgtggcggcaggcgttaagggcgcggactttatctataccgacgtgtgggtgtcgatgggcgaggccaaagagaagtgggcagagcggattgcgctgctgcgcgggtatcaggtgaacgcgcagatgatggcgctgaccgacaacccgaacgtgaagttcctgcactgtctgccggcgttccatgacgaccagactacgctcggcaagcagatggcgaaggagttcgatctgcacggcgggatggaggtgacggacgaggtgtttgagtcggcggcgagcatcgtgttcgaccaggcggaaaaccggatgcatacgattaaggcggtgatgatggcaacgcttggggagtga

SEQ ID NO: 35 - DNA, 2124 bp, Artificial Sequence
  sequence encoding Trp5
atgtcagaacaactcagacaaacatttgctaacgctaaaaaagaaaacaggaacgccttggtcacatttatgaccgcaggttacccaacagtcaaagacactgtccctattctcaagggtttccaggatggtggtgtagatatcatcgaattgggtatgcccttctctgatccaattgcagatggtcctacaattcaattatctaatactgtggctttgcaaaacggtgttaccttgcctcaaactctagaaatggtctcccaagctagaaatgaaggtgttaccgtacccataatcctaatgggttactataaccctattctaaactacggtgaagaaagatttattcaggacgctgccaaggctggtgctaatggttttatcatcgtcgatttgccaccagaggaggcgttgaaggtcagaaactacatcaatgataatggtttgagcctgatcccactagtggctccttctaccaccgacgaaagattggaattactatcgcatattgccgattcgtttgtctacgttgtgtctagaatgggtactactggtgttcaaagttctgtggccagtgatttggatgaactcatctctagagtcagaaagtacaccaaggatactcctttggccgttgggtttggtgtctctaccagagaacatttccaatcagttggtagtgttgctgacggtgtagtgattggttccaaaatcgtcacattatgtggagatgctccagagggcaaaaggtacgacgttgctaaggaatatgtacagggaattctaaatggtgctaagcataaggttctgtccaaggacgaattctttgcctttcaaaaagagtccttgaagtccgcaaacgttaagaaggaaatactggacgaatttgatgaaaatcacaagcacccaattagatttggggactttggtggtcagtatgtcccagaagctcttcatgcatgtctaagagagttggaaaagggttttgatgaagctgtcgccgatcccacattctgggaagacttcaaatccttgtattcttatattggccgtccttcttcactacacaaagctgagagattaactgagcattgtcaaggtgctcaaatctggttgaagagagaagatcttaaccacacgggatctcacaagatcaacaatgctttagcacaagttcttctagctaaaagattaggcaagaagaacgttattgctgaaaccggtgctggtcaacacggtgttgccactgccactgcatgtgctaaatttggcttaacctgtactgtgttcatgggtgcagaagatgttcgtcgccaagctttaaacgtcttcagaatgagaattctcggtgctaaagtaattgctgttactaatggtacaaagactctaagagacgctacttcagaggcattcagattttgggttactaacttgaaaactacttactacgtcgtcggttctgccattggtcctcacccatatccaactttggttagaactttccaaagtgtcattggtaaagaaaccaaggaacagtttgctgccatgaacaatggtaaattacctgacgcagttgttgcatgtgttgggggtggttccaactctacaggtatgttttcaccattcgagcatgatacttccgttaagttattgggtgtggaagccggtggtgatggtgtagatacaaagttccactctgctactctaactgccggtagacctggtgtcttccatggtgtcaagacttatgtcttgcaagatagtgatggtcaagtccatgatactcattctgtttctgctgggttagactacccaggtgtcggtccagaattggcatattggaaatctactggccgtgctcaattcattgcagctactgacgctcaggctctgcttggctttaaattattatctcaattagaaggtattattcccgctttggaatcttctcatgctgtttatggcgcttgcgaattggctaagacgatgaagcctgatcaacatttggttatcaatatttctggtagaggtgataaagatgtccaaagtgtcgctgaagtcttgccgaaattaggtccaaagataggttgggatttgagattcgaagaagacccatctgcctaa

SEQ ID NO: 36 - DNA, 2124 bp, Artificial Sequence; identical to SEQ ID NO: 35
  sequence encoding IDO

SEQ ID NO: 37 - DNA, 5691 bp, Artificial Sequence
  sequence encoding exemplary CAR GD2 ASS1+OTC without MP71 vector
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgaacggctggagctggagcctgtcgaccgagtgaagcagaccctgaactttgatctgctgaagctggccggcgacgtggagtccaaccccgggccagggaatatggccttaccagtgaccgccttgctcctgccgctggccttgctgctccacgccgccaggccggatattctgctcacacagaccccactctccctgcccgtgtcactcggggatcaggctagcatttcttgccgctcatctcagtctctggtccaccggaatgggaacacatacctccattggtacctccagaaacctggacagagccctaaactgctcatccacaaagtctcaaatcggttctccggcgtgcccgatcgctttagcggatccggatctgggaccgacttcacactgaaaatctcacgagtggaggctgaggatctcggcgtctacttctgtagtcagagtacccacgtcccacccctcacctttggcgctggaacaaaactggagctgaaacgagccgatgctgctcctaccgtgtccatctttcctggctccgggggaggcgggagcggaggcgaagtgaaactccagcagtctggcccttctctcgtggaacctggcgcttctgtgatgatctcctgtaaggcctctggatcttcctttaccggctacaacatgaactgggtccggcagaacattggcaaatccctggaatggattggcgccatcgatccttactacggcggcacatcatacaatcagaaattcaaggggcgagcaacactcactgtcgacaaatcttcatccaccgcctacatgcacctgaaatctctcacatccgaggatagtgctgtctactactgtgtctctggcatggaatactggggacagggaacttctgtcaccgtgtctagtgccaaaaccacacctccctccgtgtacggacgagtcactgtctcatctgctgaaccaaaatcctgtgacaaaacacacacatgcccaccttgtcctgcccctgaactgctcggcggaccttccgtctttctgtttccccccaaacccaaggatacactcatgatttctaggacccccgaagtcacttgtgtcgtggtcgatgtgtctcacgaggatcctgaagtgaaattcaactggtacgtggacggagtcgaggtccacaatgccaaaacaaaaccccgggaggaacagtacaatagcacctaccgagtcgtgtccgtgctcaccgtcctccatcaggattggctgaacggcaaagagtacaagtgtaaagtgagtaacaaggctctccccgctcctattgaaaaaaccatctcaaaagcaaaaggccagcctagggagcctcaggtctacacactgccaccctcacgggacgaactcaccaaaaatcaggtgtccctcacttgcctggtgaaaggcttctacccttccgatatcgctgtggaatgggagtcaaatgggcagcccgaaaacaactacaaaacaaccccccctgtgctcgattccgatggctcttttttcctgtactccaaactcaccgtggacaaatcacgctggcagcaggggaatgtcttttcttgctccgtgatgcacgaggccctccacaatcattacacccagaaatccctctcactctcacccggcaaaaaggaccctaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggcagcggcgccaccaacttcagcctgctgaagcaggccggcgacgtggaggaaaaccctggccccatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaaacttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaaggaagcggagagggcagaggaagtctgctaacatgcggtgacgtcgaggagaatcctggacctatgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggcttagcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttaa

SEQ ID NO: 38 - DNA, 4566 bp, Artificial Sequence
  sequence encoding exemplary CAR of GD2 ASS1 without MP71 vector
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgaacggctggagctggagcctgtcgaccgagtgaagcagaccctgaactttgatctgctgaagctggccggcgacgtggagtccaaccccgggccagggaatatggccttaccagtgaccgccttgctcctgccgctggccttgctgctccacgccgccaggccggatattctgctcacacagaccccactctccctgcccgtgtcactcggggatcaggctagcatttcttgccgctcatctcagtctctggtccaccggaatgggaacacatacctccattggtacctccagaaacctggacagagccctaaactgctcatccacaaagtctcaaatcggttctccggcgtgcccgatcgctttagcggatccggatctgggaccgacttcacactgaaaatctcacgagtggaggctgaggatctcggcgtctacttctgtagtcagagtacccacgtcccacccctcacctttggcgctggaacaaaactggagctgaaacgagccgatgctgctcctaccgtgtccatctttcctggctccgggggaggcgggagcggaggcgaagtgaaactccagcagtctggcccttctctcgtggaacctggcgcttctgtgatgatctcctgtaaggcctctggatcttcctttaccggctacaacatgaactgggtccggcagaacattggcaaatccctggaatggattggcgccatcgatccttactacggcggcacatcatacaatcagaaattcaaggggcgagcaacactcactgtcgacaaatcttcatccaccgcctacatgcacctgaaatctctcacatccgaggatagtgctgtctactactgtgtctctggcatggaatactggggacagggaacttctgtcaccgtgtctagtgccaaaaccacacctccctccgtgtacggacgagtcactgtctcatctgctgaaccaaaatcctgtgacaaaacacacacatgcccaccttgtcctgcccctgaactgctcggcggaccttccgtctttctgtttccccccaaacccaaggatacactcatgatttctaggacccccgaagtcacttgtgtcgtggtcgatgtgtctcacgaggatcctgaagtgaaattcaactggtacgtggacggagtcgaggtccacaatgccaaaacaaaaccccgggaggaacagtacaatagcacctaccgagtcgtgtccgtgctcaccgtcctccatcaggattggctgaacggcaaagagtacaagtgtaaagtgagtaacaaggctctccccgctcctattgaaaaaaccatctcaaaagcaaaaggccagcctagggagcctcaggtctacacactgccaccctcacgggacgaactcaccaaaaatcaggtgtccctcacttgcctggtgaaaggcttctacccttccgatatcgctgtggaatgggagtcaaatgggcagcccgaaaacaactacaaaacaaccccccctgtgctcgattccgatggctcttttttcctgtactccaaactcaccgtggacaaatcacgctggcagcaggggaatgtcttttcttgctccgtgatgcacgaggccctccacaatcattacacccagaaatccctctcactctcacccggcaaaaaggaccctaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggcagcggcgccaccaacttcagcctgctgaagcaggccggcgacgtggaggaaaaccctggccccatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaaacttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaataa

SEQ ID NO: 39 - DNA, 4398 bp, Artificial Sequence
  sequence encoding exemplary CAR of GD2 OTC without MP71 vector
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgaacggctggagctggagcctgtcgaccgagtgaagcagaccctgaactttgatctgctgaagctggccggcgacgtggagtccaaccccgggccagggaatatggccttaccagtgaccgccttgctcctgccgctggccttgctgctccacgccgccaggccggatattctgctcacacagaccccactctccctgcccgtgtcactcggggatcaggctagcatttcttgccgctcatctcagtctctggtccaccggaatgggaacacatacctccattggtacctccagaaacctggacagagccctaaactgctcatccacaaagtctcaaatcggttctccggcgtgcccgatcgctttagcggatccggatctgggaccgacttcacactgaaaatctcacgagtggaggctgaggatctcggcgtctacttctgtagtcagagtacccacgtcccacccctcacctttggcgctggaacaaaactggagctgaaacgagccgatgctgctcctaccgtgtccatctttcctggctccgggggaggcgggagcggaggcgaagtgaaactccagcagtctggcccttctctcgtggaacctggcgcttctgtgatgatctcctgtaaggcctctggatcttcctttaccggctacaacatgaactgggtccggcagaacattggcaaatccctggaatggattggcgccatcgatccttactacggcggcacatcatacaatcagaaattcaaggggcgagcaacactcactgtcgacaaatcttcatccaccgcctacatgcacctgaaatctctcacatccgaggatagtgctgtctactactgtgtctctggcatggaatactggggacagggaacttctgtcaccgtgtctagtgccaaaaccacacctccctccgtgtacggacgagtcactgtctcatctgctgaaccaaaatcctgtgacaaaacacacacatgcccaccttgtcctgcccctgaactgctcggcggaccttccgtctttctgtttccccccaaacccaaggatacactcatgatttctaggacccccgaagtcacttgtgtcgtggtcgatgtgtctcacgaggatcctgaagtgaaattcaactggtacgtggacggagtcgaggtccacaatgccaaaacaaaaccccgggaggaacagtacaatagcacctaccgagtcgtgtccgtgctcaccgtcctccatcaggattggctgaacggcaaagagtacaagtgtaaagtgagtaacaaggctctccccgctcctattgaaaaaaccatctcaaaagcaaaaggccagcctagggagcctcaggtctacacactgccaccctcacgggacgaactcaccaaaaatcaggtgtccctcacttgcctggtgaaaggcttctacccttccgatatcgctgtggaatgggagtcaaatgggcagcccgaaaacaactacaaaacaaccccccctgtgctcgattccgatggctcttttttcctgtactccaaactcaccgtggacaaatcacgctggcagcaggggaatgtcttttcttgctccgtgatgcacgaggccctccacaatcattacacccagaaatccctctcactctcacccggcaaaaaggaccctaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccacctcgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggcagcggcgccaccaacttcagcctgctgaagcaggccggcgacgtggaggaaaaccctggccccatgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggcttagcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttaa

SEQ ID NO: 40 - DNA, 342 bp, Artificial Sequence
  sequence encoding an alternative CD3z
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 41 - DNA, 786 bp, Artificial Sequence
  sequence encoding an alternative EGFRvIII target binding moiety (EGFRvIII scFv sequence derived from MR1 antibody)
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctcta

SEQ ID NO: 42 - protein, 1283 aa, Artificial Sequence
  amino acid sequence of an exemplary CAR of EGFRvIII (employing the alternative EGFRvIII target binding moiety encoded by SEQ ID NO 41) including an ASS-1 domain
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPMDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 43 - protein, 1225 aa, Artificial Sequence
  amino acid sequence of an exemplary CAR of EGFRvIII (employing the alternative EGFRvIII target binding moiety encoded by SEQ ID NO 41) including an OTC domain
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPMDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 44 - protein, 1658 aa, Artificial Sequence
  amino acid sequence of an exemplary CAR of EGFRvIII (employing the alternative EGFRvIII target binding moiety encoded by SEQ ID NO 41) including an ASS-1 domain and an OTC domain
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPMDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAKGSGEGRGSLLTCGDVEENPGPMLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

(19) United States
(12) Patent Application Publication
(10) Pub. No.: US 2020/0308557 A1
(43) Pub. Date: Oct. 1, 2020

(54) FUSION PROTEINS

(71) Applicant: CANCER RESEARCH TECHNOLOGY LIMITED, 407 St John Street (GB)

(72) Inventors: Francis Jay Mussai, Birmingham West Midlands (GB); Carmela De Santo, Birmingham West Midlands (GB); Steven Lee, Birmingham West Midlands (GB)

(73) Assignee: CANCER RESEARCH TECHNOLOGY LIMITED, London (GB)

(21) Appl. No.: 16/955,684

(22) PCT Filed: Dec. 24, 2018
(86) PCT No.: PCT/GB2018/053771

§ 371 Date: Jun. 18, 2020

(30) Foreign Application Priority Data
Dec. 22, 2017 (GB) 1721833.0

(51) Int. Cl. C12N 9/10; A61P 35/00; A61P 31/12; C12N 9/00; A61K 38/53; A61K 38/45

(52) U.S. Cl. CPC C12N 9/1018; A61P 35/00; A61P 31/12; C12Y 201/03003; A61K 38/53; A61K 38/45; C12Y 603/04005; C12N 9/93

(57) ABSTRACT

Provided are fusion target-binding proteins comprising a target binding moiety, an intracellular signalling region and a domain that promotes synthesis of arginine or an arginine precursor. The domain may be an enzyme domain such as an argininosuccinate synthase (ASS-1) enzyme domain, or an ornithine transcarbamylase (OTC) enzyme domain. Also provided are cells comprising such a fusion target-binding protein (for example cells that express the fusion target-binding protein), and nucleic acids encoding such fusion target-binding proteins. The invention also provides fusion target-binding proteins comprising a target binding moiety, an intracellular signalling region and a domain that promotes synthesis of tryptophan or a tryptophan precursor. Pharmaceutical compositions, medical uses, and methods of treatment, all using the fusion target-binding proteins, cells, or nucleic acids are disclosed. The proteins, cells, nucleic acids and pharmaceutical compositions may be used in the prevention and/or treatment of cancer, such as neuroblastoma or acute myeloid leukaemia.

### CROSS-REFERENCE TO RELATED APPLICATIONS

This application is a National Phase of International Application No. PCT/GB2018/053771 filed Dec. 24, 2018, which designated the U.S. and that International Application was published under PCT Article 21(2) in English. This application also includes a claim of priority under 35 U.S.C. § 119(a) and § 365(b) to British Application No. GB 1721833.0 filed Dec. 22, 2017, the entirety of which is hereby incorporated by reference.

### REFERENCE TO SEQUENCE LISTING

The Sequence Listing submitted Jun. 18, 2020, as a text file named “SequenceListing” created on Jan. 16, 2019 and having a size of 275 kilobytes, is hereby incorporated by reference.

### FIELD OF THE INVENTION

The present invention relates to fusion target-binding proteins, and to cells comprising such proteins. It also relates to nucleic acids encoding fusion target-binding proteins. The invention relates to pharmaceutical compositions, medical uses, and methods of treatment, all using the fusion target-binding proteins, cells, or nucleic acids disclosed.

### BACKGROUND

Fusion proteins with target-binding capabilities have been used in a number of therapeutic applications. Most notably, T cells engineered to express chimeric antigen receptors (CARs) have been used in the treatment of cancer. However, as discussed further below, despite showing considerable clinical promise, such treatments have not been universally effective.

### CAR-T Failure in Pre-Clinical and Clinical Studies

Despite advances in cytotoxic chemotherapy for both adult and paediatric cancers, it is clear that a number of major cancer subtypes still have an extremely poor prognosis. Immune therapies provide an alternative approach to targeting the malignant cancer cells directly, and avoid the toxic side-effects to normal cells of standard approaches.

Chimeric Antigen Receptor (CAR)-T cells (CAR-T) are autologous patient-derived T cells which have been engineered, typically with an antibody fragment (scFv), to specifically recognise surface antigens on tumour cells. The proof-of-principle of using CAR-T cells to successfully treat paediatric cancers has been established in patients with chemo-resistant, relapsed paediatric B Acute Lymphoblastic Leukaemia who underwent rapid and sustained remissions using anti-CD19 CAR T cells. In solid tumours neuroblastoma, the most common solid cancer of childhood, has been the model of choice and proved highly informative in the response of solid tumours to CAR-T cell therapy. Preclinical studies indicate that CAR T cells that recognise disialoganglioside 2 (GD2) antigen could represent a powerful new way of killing neuroblastoma cells. Although neuroblastoma has become the paradigm for CAR-T cell development against solid tumours, only limited anti-tumour efficacy has been seen in preclinical models and early phase trials. First generation anti-GD2 CAR T cells failed to persist in vivo and had minimal anti-tumour effects. Second generation anti-GD2-CAR T cells (with CD28 or 4-1BB costimulatory domains) had improved persistence in vivo, leading to moderate tumour regressions, but become functionally exhausted in the presence of neuroblastoma. In humans, a study of anti-GD2 CAR T cells made the key observation that despite infusion of large numbers of these cells, CAR T cell numbers become low or undetectable within weeks, and that the majority of patients with active disease did not achieve a complete remission. Importantly patients who had low-level persistence of CAR T cells had a longer survival. These findings suggest that the local and systemic tumour microenvironment impairs persistence of CAR-T cells, despite the presence of large target antigenic load on residual neuroblastoma tumours.

CAR-T cell therapy has also been tested against a limited number of other solid tumours in vitro, in vivo, and in man. In each case results against these malignancies have failed to replicate the exciting data found for anti-CD19 CAR-T cells in ALL.

### Acute Myeloid Leukaemia

Acute Myeloid Leukaemia, is the most common acute leukaemia of adults and the second most common leukaemia of childhood. Incidence increases with age, and for patients with high risk or relapsed disease the prognosis is extremely poor with survival <12 months in adults, despite haematopoietic stem cell transplant. For elderly patients or those with co-existing morbidities standard chemotherapeutic regimens are poorly tolerated leading to sub-optimal treatment, and an in ability to achieve cure. Few effective new drugs have been developed for AML, as such immunotherapeutics offers the potential of a different approach. CD33 is almost universally expressed on AML blasts and has proved to be an effective target for immunotoxin-based therapeutics (Gemtuzumab ozogamicin). Anti-CD33 CAR-T cells are cytotoxic to AML blasts in vitro and eradicate leukaemic burden in vivo. On this basis a Phase I clinical trial of anti-CD33 CAR T cells has been initiated in China (NCT01864902 and NCT02958397). Reports from 1 patient with chemo-refractory AML showed a reduction in bone marrow AML blasts. These results provide proof-of principle that anti-CD33 CAR T cells can be effective. However disease relapsed by 9 weeks post CAR infusion despite measurable CAR-T cells remaining in both the blood and bone marrow. The finding suggests that the CAR-T cells have been rendered inactive, by the leukaemia microenvironment (no evidence for CD33 loss on AML blasts as a mechanism of escape).

### Mesothelioma, Ovarian and Pancreatic Cancer

Mesothelioma, an asbestos related tumour with almost universally poor prognosis in adults, expresses the cell surface glycoprotein mesothelin. Mesothelin is also expressed on epithelial cancers, such as ovarian, lung adenocarcinoma, and pancreatic cancer. Mesothelin has been demonstrated to be an effective and selective target for passive immunotherapy with immunotoxins such as SS1P leading to its choice for development in CAR T technologies. In murine models anti-mesothelin CAR-T cells demonstrate clear and persistent anti-tumour activity. Anti-mesothelin CAR-T cells have also been administered to patients with these tumours and although limited responses were detected (PR, SD) in each case the tumours progressed. CAR-T cell persistence was extremely poor with cells becoming undetectable within only days of initial or repeat administration. Even when CAR-T cells are placed within the tumour, and hence in close proximity to target antigen, responses remain muted suggesting a strong immunosuppressive microenvironment that reduces the function of the T cells.

### Glioblastoma

Glioblastoma is one of the most devastating brain tumours of both adults and children, with patients frequently experiencing a rapid disease progression and treatment failure despite intensive chemotherapy and radiotherapy based regimens. Glioblastomas express a variant of the Epidermal Growth Factor Receptor—EGFRvIII, providing a tumour-specific antigen which can be targeted by immunotherapy. EGFRvIII may also be expressed on approximately one third of advanced colorectal cancers. Anti-EGFRvIIII CAR-T cells demonstrated disease control of glioblastomas in orthotopic murine xenografts. However in all cases tumours continue to grow, leading to murine death, despite detectable levels of CAR-T cells in all organs including the brain. Again this data suggests that the CAR-T cells are inactivated by the tumour microenvironment. A Phase I trial based on this rationale is currently underway (NCT02844062, NCT02664363).

### Arginine and the Immunosuppressive Microenvironment

Arginine is a semi-essential amino acid, required by healthy tissues for a number of cell processes including cell viability, proliferation and protein synthesis. Whole body arginine levels are maintained principally through dietary intake, and to a lesser extent by synthesis from precursors in an ‘intestinal-renal axis’. At a cellular level, arginine is imported from the extracellular fluid via Cationic Amino Acid (CAT; SLC7A) family of transporters and enters the urea cycle. In conditions of high demand such as inflammation, pregnancy, and cancer, arginine levels can become limited in the local tissue microenvironment and systemically. Some tissues and cells can protect themselves by resynthesizing arginine from precursors, through the expression of ArgininoSuccinate Synthase (ASS1) and Ornithine Transcarbamylase (OTC). Cells which lack expression of at least one of these enzymes are dependent on import of extracellular arginine, a state known as arginine auxotrophism.

Previous studies have suggested that inhibition of arginase at the tumour site may be beneficial in addressing the issues of poor CAR T cell activity in vivo.

### SUMMARY OF THE INVENTION

In a first aspect, the invention provides a fusion target-binding protein comprising a target binding moiety, an intracellular signalling region and a domain that promotes synthesis of arginine or an arginine precursor. For the sake of brevity, fusion target-binding proteins in accordance with the various aspects and embodiments of the invention will be referred to herein as “proteins of the invention”.

In a second aspect, the invention provides a cell comprising a fusion target-binding protein comprising a target binding moiety, an intracellular signalling region and a domain that promotes synthesis of arginine or an arginine precursor. The cell may express the fusion target-binding protein.

In a third aspect, the invention provides a nucleic acid encoding a fusion target-binding protein comprising a target binding moiety, an intracellular signalling region and a domain that promotes synthesis of arginine or an arginine precursor. It will be appreciated that nucleic acids in accordance with the third aspect of the invention may be expressed to yield a fusion target-binding protein in accordance with the first aspect of the invention or a cell in accordance with the second aspect of the invention.

In a fourth aspect, the invention provides a fusion target-binding protein comprising a target binding moiety, an intracellular signalling region and a domain that promotes synthesis of tryptophan or a tryptophan precursor.

In a fifth aspect, the invention provides a cell comprising a fusion target-binding protein comprising a target binding moiety, an intracellular signalling region and a domain that promotes synthesis of tryptophan or a tryptophan precursor. The cell may express the fusion target-binding protein.

In a sixth aspect, the invention provides a nucleic acid encoding a fusion target-binding protein comprising a target binding moiety, an intracellular signalling region and a domain that promotes synthesis of tryptophan or a tryptophan precursor. It will be appreciated that nucleic acids in accordance with the sixth aspect of the invention may be expressed to yield a fusion target-binding protein in accordance with the fourth aspect of the invention or a cell in accordance with the fifth aspect of the invention.

In a seventh aspect, the invention provides a pharmaceutical composition comprising a fusion target-binding protein or cell in accordance with the first, second, fourth or fifth aspect of the invention, or a nucleic acid in accordance with the third or sixth aspect of the invention.

In an eighth aspect, the invention provides a fusion target-binding protein in accordance with the first or fourth aspect of the invention for use as a medicament.

In a ninth aspect, the invention provides a cell in accordance with the second or fifth aspect of the invention for use as a medicament.

In a tenth aspect, the invention provides a nucleic acid in accordance with the third or sixth aspect of the invention for use as a medicament.

In an eleventh aspect, the invention provides a pharmaceutical composition in accordance with the seventh aspect of the invention for use as a medicament.

In a twelfth aspect, the invention provides a method of preventing and/or treating a disease in a subject in need of such prevention and/or treatment, the method comprising providing the subject with a fusion target-binding protein of the invention. The fusion target-binding protein may be in accordance with the first, fourth, or eleventh aspect of the invention. The protein of the invention may be provided as part of a cell of the invention, such as a cell of the second or fifth aspects of the invention.

As discussed further elsewhere in the specification, the fusion target-binding proteins, cells, nucleic acids, and pharmaceutical compositions of the invention may be used in the prevention and/or treatment of one or more disorders selected from the group consisting of: cancer; infections, such as viral infections; and autoimmune diseases.

### BRIEF DESCRIPTION OF THE DRAWINGS

FIG. 1. Illustrates the optimisation of fusion target-binding protein containing viral titres. Panel A shows concentrations of retroviral particles increased over 72 hours in the supernatants of AMPHO Phoenix cells. Panel B shows fusion target-binding protein T cell transduction efficiency was assessed by flow cytometry detection of tCD34. No difference in transduction efficiency of PBMCs was seen using AMPHO cell line supernatants

FIG. 2. Shows that arginine pathway enzymes demonstrate activity in transduced Jurkat cells. Panel A shows protein-enzyme constructs can be produced to a high degree of purity, assessed by measuring expression of tCD34 using flow cytometry. Panel B illustrates an increase in the expression of ASS-1 and OTC in the transduced cells. Panel C illustrates the ability of the domains that promote the synthesis of arginine or an arginine precursor in the transduced cells to perform their function. Panel D shows the results of a study of cellular catabolism of ornithine into citrulline (by OTC). The amount of citrulline produced by cells expressing fusion target-binding proteins containing OTC enzyme domains (“GD2-OTC”), or fusion target-binding proteins containing both ASS-1 and OTC enzyme domains (“GD2-ASS-OTC”), was assessed and compared to cells expressing control constructs without an OTC enzyme domain (“GD2”), or fusion target-binding proteins containing an ASS-1 enzyme domain (“GD2-ASS”). Panel E shows the persistence of fusion target-binding protein T cells transduced with constructs comprising domains (ASS-1) that promote the synthesis of the arginine precursor argininosuccinate in a tumour microenvironment in mice. Panel F shows that fusion target-binding protein T cells comprising an OTC domain (GD2-OTC) showed a significantly enhanced persistence compared to T cells without the fusion target-binding protein comprising the OTC domain (GD2 only).

FIG. 3. Illustrates that arginine pathway enzymes can be transduced into PBMCs from human donor cells. Panel A shows that the fusion target-binding protein-enzyme constructs can be produced to a high degree of purity in the PBMCs, by measuring expression of tCD34 using flow cytometry. Panel B shows an increase in expression of ASS-1 and OTC in transduced cells. Panel C shows that there were no differences in expression of the co-inhibitory receptors LAG-3, TIM-3, and PD-1 in fusion target-binding protein T cells also containing the constructs comprising an enzyme domain that promotes synthesis of arginine or an arginine precursor. Panel D shows persistence of PBMCs transduced with the constructs comprising a domain that promotes the synthesis of arginine or an arginine precursor, measured during a 7 day expansion, as detected by flow cytometry of tCD34.

FIG. 4. Shows ASS-1 and OTC enzyme domains confer a significant metabolic and proliferative advantage in low arginine tumour conditions. Panel A shows fusion target-binding protein T cells comprising ASS-1 enzyme domain (GD2-ASS) and OTC enzyme domain GD2-OTC) to enhance citrulline metabolism when cultured in normal arginine and 75% arginine depleted media conditions, detected by ELISA of culture supernatants. Panel B illustrates specific cell lysis of neuroblastoma and myeloenous leukaemia cell lines by T cells expressing fusion target-binding proteins of the invention (comprising an ASS-1 domain (“GD2-ASS”), or an OTC domain (“GD2-OTC”)) as assessed against lysis by T cells expressing a control protein (“GD2-BB” without ASS-1 or OTC). Panel C shows fusion target-binding protein T cells comprising ASS-1 or OTC enzyme domain showed a significant rescue of proliferation in low arginine conditions compared to the control (GD2 without enzyme). The conditions shown on the graph are, normal arginine (RPMI+10% FCS), neuroblastoma-derived low arginine supernatants (Lan-1 TCM), or 75% arginine deplete media.

FIG. 5. Shows modified fusion target-binding protein T cells have enhanced anti-tumour activity in vivo and can be applied to non-GD2 fusion target-binding protein T cells. Panel A show the relative tumour volume of NOG-SCID mice engrafted with GD2+ tumour cells and administered with the fusion target-binding protein T cells comprising an ASS-1 domain (GD2-ASS) and without (GD2 only). The relative tumour growth was measured over time. Panel B shows the percentage survival of the mice following administration with GD2-ASS-land GD2 only fusion target-binding protein T cells. Panel C illustrates the viability of CD33 and CD33-ASS-1fusion target-binding protein T cells in 50%-75% AML cell line condition media (low in arginine) or 75% arginine depleted media.

FIG. 6. Shows increased persistence of cells of the invention in arginine-depleted conditions in vivo. Demonstrated in NOG-SCID mice engrafted with 5×10⁶ anti-GD2 CAR-T Jurkat cells (control cells), or Jurkat cells expressing proteins of the invention comprising a GD2 target moiety and an ASS-1 domain (GD2-ASS), or cells expressing proteins of the invention comprising a GD2 target moiety and an OTC domain (GD2-OTC), administered intravenously. The GD2-ASS-1 and GD2-OTC CAR-T cells showed significantly enhanced persistence as compared to the control cells comprising the unmodified GD2 CAR-T construct.

FIG. 7. Shows arginine pathway enzymes can be transduced into PBMCs from human donors comprising various target binding moieties. Western blots show that expression of ASS-1 and OTC is increased in PBMC cells transduced with proteins of the invention compared to a control (panel A). Panel B shows expressions of LAG-3, TIM-3, and PD-1 assessed by flow cytometry.

FIG. 8. Shows the cytocidal activity of CAR T cells expressing a CD33 targeting domain in combination with either an ASS-1 domain, an OTC domain or an ASS-1 and OTC domain, cultured in the presence of K562 leukaemia cells at difference effector to target rations for 4 hours. Cells comprising proteins of the invention maintain cytocidal activity.

FIG. 9. Shows CAR T cells expressing proteins of the invention comprising either anti-GD2, anti-CD33, anti-mesothelin, or anti EGFRvIII targeting moiety in combination with either an ASS-1 domain, an OTC domain or an ASS-1 and OTC domain confer a significant metabolic and proliferative advantage in low arginine tumour conditions compared to control cells. Unmodified CAR-T cells sharing the same binding domains (i.e. anti-GD2, anti-CD33, anti-mesothelin, or anti EGFRvIII), but lacking the enzyme domains, were used as controls CAR T cells expressing proteins of the invention.

FIG. 10. Shows cells expressing proteins of the invention comprising a GD2-binding moiety in combination with either: an ASS-1 domain, an OTC domain, or an ASS-1 and OTC domain, cultured in tumour conditioned media (TCM) confer a significant metabolic and proliferative advantage (panel A). Panel B shows that cells expressing proteins of the invention comprising a CD33-binding moiety in combination with either: an ASS-1 domain, an OTC domain, or an ASS-1 and OTC domain, cultured in tumour conditioned media (TCM) also confer a significant metabolic and proliferative advantage.

FIG. 11. Shows CAR-T cells expressing a protein of the invention comprising an ASS-1 domain (Anti-CD33-ASS-1CAR-T cells) significantly enhance AML clearance from the bone marrow of leukaemia bearing mice. This data relates to clearance of HL-60 acute myeloid leukaemia (AML) cells engrafted into NOG-SCID mice.

FIG. 12. Panel A illustrates increased persistence of the cells of the invention within the spleens of neuroblastoma xenograft mice treated with cells expressing a protein of the invention comprising a GD2-binding moiety and an ASS-1 domain. Improved ability of the cells of the invention to proliferate in response to antigen stimulation is demonstrated in Panel B.

FIG. 13. Panel A illustrates increased persistence of the cells of the invention within the spleens of AML xenograft mice were treated with cells expressing a protein of the invention comprising a CD33-binding moiety and one of: an ASS-1 domain, an OTC domain; or an ASS-1 domain and an OTC domain. Improved ability of the cells of the invention to proliferate in response to antigen stimulation is demonstrated in Panel B.

FIG. 14. Sets out details of constructs, comprising nucleic acids of the invention, that have been used in successful lentiviral production of cells of the invention.

### DETAILED DESCRIPTION OF THE INVENTION

The present invention is based upon the inventor's recognition that cells expressing fusion target-binding proteins that incorporate a domain that promotes synthesis of arginine or an arginine precursor (and/or a domain that promotes synthesis of tryptophan or a tryptophan precursor) exhibit significant advantages in vivo. In particular, the inventors have found that cells expressing such proteins are able to overcome the immunosuppressive effects associated with the tumour microenvironment, which the inventors believe have contributed to the failures of many prior art CAR-based therapies.

One of the particular advantages exhibited by cells expressing proteins of the invention is their increased persistence and proliferation in the tumour microenvironment. It is known that this microenvironment can otherwise dramatically reduce effectiveness of CAR T cells of the prior art.

Furthermore, cells expressing proteins of the invention exhibit an improved ability to proliferate in the conditions of the immunosuppressive tumour microenvironment. Proliferation of cells expressing CARs is usually dramatically inhibited by arginine-depleted conditions.

Both of these advantages allow for improved treatments, in which the numbers of cells expressing proteins of the invention are increased, and these cells have prolonged residency at the tumour site, thus allowing improved killing of cancer cells. In this respect, it is important to note that the modifications made in respect of the proteins and cells of the invention do not significantly detract from their ability to kill cancer cells (whether by cytotoxic action, or specific cell lysis).

Accordingly, it will be recognised that the proteins and cells of the invention provide improved therapeutic agents as compared to CAR-based therapies of the prior art. The various aspects and embodiments of the invention described herein arise from, or contribute to, these improvements.

For the purposes of understanding the invention, it will now be further described with reference to the following definitions. For the sake of brevity the paragraphs that follow may refer to particular embodiments only in the context of proteins of the invention, but it will be appreciated that, except for where the context requires otherwise, embodiments referred to in connection with proteins of the invention may be employed in any of the other aspects of the invention disclosed herein.

### Fusion Target-Binding Proteins

Fusion target-binding proteins are artificial fusion proteins that enable a desired specificity to be conferred on desired biological properties of a cell by which the protein of the invention is expressed. For the sake of brevity, they will also be referred to as “proteins” or “proteins of the invention” in the present disclosure. Different types of cells, and the desired biological properties that they are respectively able to provide in the context of the present invention, are discussed further elsewhere in the specification. Typically, in the context of medical uses of fusion target-binding proteins and cells expressing such proteins, cytocidal activity targeted against cells associated with a disease (such as cancer cells or infected cells) confers the required therapeutic utility.

Proteins of the invention comprise at least a target binding moiety, an intracellular signalling region and a domain that promotes synthesis of arginine or an arginine precursor and/or a domain that promotes synthesis of tryptophan or a tryptophan precursor. These terms are defined elsewhere within the present specification. The skilled person will appreciate that such proteins may also incorporate various other optional domains or regions.

The different portions of the fusion target-binding protein (target binding moieties, intracellular signalling regions, and domains that promote synthesis of arginine or an arginine precursor and/or domains that promote synthesis of tryptophan or a tryptophan precursor) may be derived from two or more different “sources”. Thus, the different portions may be derived from two or more naturally occurring molecules, such as proteins. Additionally, the different portions may be derived from different sources in terms of different originating kingdoms or species.

A class of fusion target-binding proteins of particular interest in the context of the present invention are chimeric antigen receptor (CAR) proteins. CARs utilise antibodies, or fragments thereof, to confer specificity of binding, and intracellular signalling regions to determine the specific biological activity required. Various different generations of CARs are known, and each of these different generations represents a suitable example of a fusion target-binding protein of the invention, unless the context of the present disclosure requires otherwise.

For the avoidance of doubt, proteins of the invention may also be taken as encompassing T cell receptors (TCRs) modified to comprise a domain that promotes synthesis of arginine or an arginine precursor and/or a domain that promotes synthesis of tryptophan or a tryptophan precursor. In such embodiments, the target binding moiety may be provided by the TCR α and TCR β chains of the receptor. Since the target binding moiety and domain that promotes synthesis of arginine or an arginine precursor and/or a domain that promotes synthesis of tryptophan or a tryptophan precursor are from different sources, such modified TCRs are chimeric for the purposes of the present invention.

Proteins of the invention typically further comprise additional portions, including one or more from the group consisting of: a transmembrane portion, a CH2CH3 spacer portion, a CD8 hinge portion, and a CD8a signalling portion.

The amino acid sequences of exemplary proteins of the invention are set out in SEQ ID NOs: 12 to 23. It will be appreciated that a molecule comprising or consisting of any of these sequences represents a protein in accordance with the first aspect of the invention. Any of the proteins set out in SEQ ID NOs: 12 to 23 may be utilised in the medical uses, methods of treatment, or pharmaceutical compositions of the invention.

### Fragments or Variants of the Sequences of the Exemplary Proteins of the Invention

The specification contains a number of exemplary protein and nucleic acid sequences. As well as the fusion target-binding proteins and nucleic acids encoding them, these include sequences of target binding moieties, intracellular signalling regions, and enzyme domains.

It will be appreciated that, except for where the context requires otherwise, the scope of the invention should not be limited to the specific exemplary sequences set out herein. In particular, the skilled reader will recognise that fragments or variants of the exemplary sequences may still be able to provide the required activity conferred by the exemplary sequences. Such suitable fragments or variants of the exemplary sequences may be utilised in the various aspects and embodiments of the invention.

Accordingly, references in the present specification to exemplary amino acid or nucleic acid sequences should, except for where the context requires otherwise, be taken as also encompassing functional fragments or variants of the exemplary sequences. For example, a suitable fragment may comprise at least 60%, at least 65%, at least 70%, at least 75%, at least 80%, at least 85%, at least 90%, or at least 95% of the full length of a relevant exemplary sequence. Indeed, a suitable variant may comprise at least 96%, at least 97%, at least 98%, or at least 99% of the full length of the exemplary sequence.

A suitable variant may share at least 60%, at least 65%, at least 70%, at least 75%, at least 80%, at least 85%, at least 90%, or at least 95% identity with a relevant exemplary sequence. Indeed, a suitable variant may share at least 96%, at least 97%, at least 98%, or at least 99% identity with the relevant exemplary sequence.

That a fragment or variant is “functional” may be assessed experimentally, with reference to assays known to those skilled in the art, including those assays described in the Examples.

### Arginine or an Arginine Precursor

The present invention relates to fusion target-binding proteins that comprise a domain that promotes synthesis of arginine or an arginine precursor.

Arginine (frequently abbreviated to “Arg” or “R”) is a semi-essential amino acid. It has a molecular mass of 174.2 g/mol-1, and may also be referred to as 2-amino-5-guanidinopentanoic acid.

In the context of the present disclosure, references to arginine precursors should be construed in the context of the arginine pathway, the series of chemical reactions by which metabolic arginine is imported, catabolised, or recycled. Thus, a precursor of arginine may, for the present purposes, be taken as encompassing any compound that is either directly or indirectly converted to arginine.

A Domain that Promotes Synthesis of Arginine or an Arginine Precursor

The proteins of the invention comprise a domain that promotes synthesis of arginine or an arginine precursor. The ability of a domain to fulfil this function, that is to say to promote synthesis of arginine or an arginine precursor, may be investigated by any suitable means or assay.

The skilled person will appreciate that a suitable means or assay may be selected with reference to the compound, for example arginine or an arginine precursor, synthesis of which is to be promoted. Merely by way of example, suitable assays by which the ability of a domain to promote the requisite synthesis are described further in the Examples, in relation to the characterisation of exemplary cells of the invention.

Suitably the domain that promotes synthesis of arginine or an arginine precursor may be an enzyme domain capable of promoting the synthesis of arginine or an arginine precursor. In such an embodiment the enzyme domain may comprise the full length enzyme domain, or a fragment or a variant of such a domain, as long as the domain exhibits the requisite activity.

In a suitable embodiment, the arginine precursor, synthesis of which is promoted, is arginosuccinate. In such an embodiment, the domain that promotes such synthesis may be selected from; an argininosuccinate synthase (ASS-1) enzyme domain; and argininosuccinate sythetase (ArgG) domain. Suitably, the domain that promotes such synthesis is an argininosuccinate synthase (ASS-1) enzyme domain.

In a suitable embodiment, the arginine precursor, synthesis of which is promoted, is arginosuccinate. Catalysis of argininosuccinate, such as by argininosuccinate lyase yields arginine. In such an embodiment, the domain that promotes such synthesis may be selected from; an argininosuccinate lyase (ASL) enzyme domain; and argininosuccinate lyase (ArgH) enzyme domain.

In a suitable embodiment, the arginine precursor, synthesis of which is promoted, is citrulline. In such an embodiment, the domain that promotes such synthesis may be selected from; an ornithine transcarbamylase (OTC) enzyme domain; ornithine decarboxylase (ODC1); and an ornithine carbamoyltransferase (ArgF) enzyme domain. Suitably, the domain that promotes such synthesis is an ornithine transcarbamylase (OTC) enzyme domain.

Accordingly, in a suitable embodiment the domain that promotes synthesis of arginine or an arginine precursor comprises an enzyme domain selected from the group consisting of: an ASS-1 domain; an OTC domain; an ASL domain; an OCD1 domain; an ArgG domain; an ArgH domain; and an ArgF domain. The domain may be selected from an ASS-1 domain and/or an OTC domain.

Suitably a protein in accordance with the invention may comprise a plurality of domains that promote synthesis of arginine or an arginine precursor. Suitably such a plurality may comprise a plurality of enzyme domains. The plurality may comprise more than one copy of an individual enzyme domain, and/or a combination of multiple enzyme domains. For example, a protein in accordance with the invention may comprise a combination of enzyme domains selected from the group consisting of: an ASS-1 domain; an OTC domain; an ASL domain; an OCD1 domain an ArgG domain; an ArgH domain; and ArgF domain. Merely by way of example, a protein of the invention may comprise both an ASS-1 domain and an OTC domain.

A suitable enzyme domain that promotes synthesis of arginine or an arginine precursor may be a human enzyme domain. In such an embodiment, this may be ASS-1, OTC, ASL or ODC1.

A suitable enzyme domain that promotes synthesis of arginine or an arginine precursor may be a naturally occurring enzyme domain. Alternatively, a suitable enzyme domain that promotes synthesis of arginine or an arginine precursor may be a fragment or derivative of a naturally occurring enzyme domain that is able to recapitulate the synthetic activity of the naturally occurring domain. Such a fragment or derivative may have synthetic activity that is 50%, or more, of that of the naturally occurring domain; 60%, or more, of that of the naturally occurring domain; 70%, or more, of that of the naturally occurring domain; 80%, or more, of that of the naturally occurring domain; 90%, or more, of that of the naturally occurring domain; or 95%, or more, of that of the naturally occurring domain. Indeed, a suitable fragment or derivative may have greater synthetic activity than the naturally occurring domain from which it is derived, which is to say it may have 100%, or more, of the synthetic activity of the naturally occurring domain.

Further details of suitable enzyme domains which promote synthesis of an arginine precursor for the purposes of the present disclosure, are set out below.

### An ASS-1 Enzyme Domain

An example of an ASS-1 enzyme domain suitable for incorporation in the proteins of the invention is set out in SEQ ID NO: 1.

Alternatively, a fragment or derivative of the sequence set out in SEQ ID NO: 1 may be used as a suitable ASS-1 enzyme domain for incorporation in a protein of the invention.

Any enzyme domain that confers ASS-1 activity on the proteins of the invention may be used in such an embodiment. ASS-1 activity may be assessed by an assay described in the Examples section of the disclosure.

A suitable level of ASS-1 activity may be equivalent to that conferred by SEQ ID NO: 1. Alternatively, enzyme domains conferring lower or higher levels of ASS-1 activity may be still of benefit.

A suitable fragment of the ASS-1 enzyme domain set out in SEQ ID NO: 1 may comprise all but 1 of the amino acid residues set out in SEQ ID NO: 1, all but 2 of the amino acid residues set out in SEQ ID NO: 1, all but 3 of the amino acid residues set out in SEQ ID NO: 1, all but 4 of the amino acid residues set out in SEQ ID NO: 1, all but 5 of the amino acid residues set out in SEQ ID NO: 1, all but 6 of the amino acid residues set out in SEQ ID NO: 1, all but 7 of the amino acid residues set out in SEQ ID NO: 1, all but 8 of the amino acid residues set out in SEQ ID NO: 1, all but 9 of the amino acid residues set out in SEQ ID NO: 1, or all but 10 of the amino acid residues set out in SEQ ID NO: 1.

Merely by way of example, a suitable variant of the sequence set out in SEQ ID NO: 1 may share at least 75% identity with SEQ ID NO: 1, or with a fragment of SEQ ID NO: 1 as defined above. A suitable variant of may share at least 80% identity with SEQ ID NO: 1, or a fragment thereof; at least 85% identity with SEQ ID NO: 1, or a fragment thereof; at least 90% identity with SEQ ID NO: 1, or a fragment thereof; at least 95% identity with SEQ ID NO: 1, or a fragment thereof; at least 96% identity with SEQ ID NO: 1, or a fragment thereof; at least 97% identity with SEQ ID NO: 1, or a fragment thereof; at least 98% identity with SEQ ID NO: 1, or a fragment thereof; or at least 99% identity with SEQ ID NO: 1, or a fragment thereof. In order to be suitable for incorporation in the CARs of the invention, such variants should retain synthetic activity of ASS-1 as referred to above.

A suitable ASS-1 enzyme domain may provide at least 50% of the activity of that provided by the domain of SEQ ID NO: 1. Suitably, it may provide at least 60%, at least 70%, at least 80%, at least 90% of the activity of that provided by the domain of SEQ ID NO: 1. Suitably, it may even provide at least 100% of the activity of that provided by the domain of SEQ ID NO: 1.

In fact, a suitable ASS-1 domain may provide at least 110% of the activity of that provided by the domain of SEQ ID NO: 1. Suitably, it may provide at least 120%, at least 130%, at least 140%, at least 150%, at least 160%, at least 170%, at least 180%, at least 190% of the activity of that provided by the domain of SEQ ID NO: 1. Suitably, it may even provide at least 200% of the activity of that provided by the domain of SEQ ID NO: 1.

### An OTC Enzyme Domain

An example of an OTC enzyme domain suitable for incorporation in the proteins of the invention is set out in SEQ ID NO: 2.

Alternatively, a fragment or derivative of the sequence set out in SEQ ID NO: 2 may be used as a suitable OTC enzyme domain for incorporation in a protein of the invention.

Any enzyme domain that confers OTC activity on the proteins of the invention may be used in such an embodiment. OTC activity may be assessed by an assay described in the Examples section of the disclosure.

A suitable level of OTC activity may be equivalent to that conferred by SEQ ID NO: 2. Alternatively, enzyme domains conferring lower or higher levels of OTC activity may be still of benefit.

A suitable fragment of the OTC enzyme domain set out in SEQ ID NO: 2 may comprise all but 1 of the amino acid residues set out in SEQ ID NO: 2, all but 2 of the amino acid residues set out in SEQ ID NO: 2, all but 3 of the amino acid residues set out in SEQ ID NO: 2, all but 4 of the amino acid residues set out in SEQ ID NO: 2, all but 5 of the amino acid residues set out in SEQ ID NO: 2, all but 6 of the amino acid residues set out in SEQ ID NO: 2, all but 7 of the amino acid residues set out in SEQ ID NO: 2, all but 8 of the amino acid residues set out in SEQ ID NO: 2, all but 9 of the amino acid residues set out in SEQ ID NO: 2, or all but 10 of the amino acid residues set out in SEQ ID NO: 2.

Merely by way of example, a suitable variant of the sequence set out in SEQ ID NO: 2 may share at least 75% identity with SEQ ID NO: 2, or with a fragment of SEQ ID NO: 2 as defined above. A suitable variant of may share at least 80% identity with SEQ ID NO: 2, or a fragment thereof; at least 85% identity with SEQ ID NO: 2, or a fragment thereof; at least 90% identity with SEQ ID NO: 2, or a fragment thereof; at least 95% identity with SEQ ID NO: 2, or a fragment thereof; at least 96% identity with SEQ ID NO: 2, or a fragment thereof; at least 97% identity with SEQ ID NO: 2, or a fragment thereof; at least 98% identity with SEQ ID NO: 2, or a fragment thereof; or at least 99% identity with SEQ ID NO: 2, or a fragment thereof. In order to be suitable for incorporation in the CARs of the invention, such variants should retain synthetic activity of OTC as referred to above.

A suitable OTC enzyme domain may provide at least 50% of the activity of that provided by the domain of SEQ ID NO: 2. Suitably, it may provide at least 60%, at least 70%, at least 80%, at least 90% activity of the activity of that provided by the domain of SEQ ID NO: 2. Suitably, it may even provide at least 100% of the activity of that provided by the domain of SEQ ID NO:

2.

In fact, a suitable OTC domain may provide at least 110% of the activity of that provided by the domain of SEQ ID NO: 2. Suitably, it may provide at least 120%, at least 130%, at least 140%, at least 150%, at least 160%, at least 170%, at least 180%, at least 190% of the activity of that provided by the domain of SEQ ID NO: 2. Suitably, it may even provide at least 200% of the activity of that provided by the domain of SEQ ID NO: 2.

### An Arginosuccinate Lyase (ASL) Enzyme Domain

An example of an ASL enzyme domain suitable for incorporation in the proteins of the invention is encoded by the nucleic acid sequence set out in SEQ ID NO: 30.

Alternatively, a fragment or derivative of the sequence set out in SEQ ID NO: 30 may be used to encode a suitable ASL enzyme domain for incorporation in a protein of the invention.

Any enzyme domain that confers ASL activity on the protein of the invention may be used in such an embodiment.

A suitable level of ASL activity may be equivalent to that conferred by a protein encoded by SEQ ID NO: 30 Alternatively, enzyme domains conferring lower or higher levels of ASL activity may be still of benefit.

### An Ornithine Decarboxylase (ODC1) Enzyme Domain

An example of an ODC1 enzyme domain suitable for incorporation in the proteins of the invention is encoded by the nucleic acid sequence set out in SEQ ID NO: 31.

Alternatively, a fragment or derivative of the sequence set out in SEQ ID NO: 31 may be used to encode a suitable ODC1 enzyme domain for incorporation in a protein of the invention.

Any enzyme domain that confers ODC1 activity on the proteins of the invention may be used in such an embodiment.

A suitable level of ODC1 activity may be equivalent to that conferred by a protein encoded by SEQ ID NO: 31. Alternatively, enzyme domains conferring lower or higher levels of ODC1 activity may be still of benefit.

### An Arginosuccinate Synthetase (ArgG) Enzyme Domain

An example of an ArgG enzyme domain suitable for incorporation in the CARs of the invention is encoded by the nucleic acid sequence set out in SEQ ID NO: 32.

Alternatively, a fragment or derivative of the sequence set out in SEQ ID NO: 32 may be used to encode a suitable ArgG enzyme domain for incorporation in a protein of the invention.

Any enzyme domain that confers ArgG activity on the proteins of the invention may be used in such an embodiment.

A suitable level of ArgG activity may be equivalent to that conferred by a protein encoded by SEQ ID NO: 32 Alternatively, enzyme domains conferring lower or higher levels of ArgG activity may be still of benefit.

### An Arginosuccinate Lyase (ArgH) Enzyme Domain

An example of an ArgH enzyme domain suitable for incorporation in the proteins of the invention is encoded by the nucleic acid sequence set out in SEQ ID NO: 33.

Alternatively, a fragment or derivative of the sequence set out in SEQ ID NO: 33 may be used to encode a suitable ArgH enzyme domain for incorporation in a protein of the invention.

Any enzyme domain that confers ArgH activity on the proteins of the invention may be used in such an embodiment.

A suitable level of ArgH activity may be equivalent to that conferred by a protein encoded by SEQ ID NO: 33 Alternatively, enzyme domains conferring lower or higher levels of ArgH activity may be still of benefit.

### An Ornithine Carbamoyltransferase (ArgF) Enzyme Domain

An example of an ArgF enzyme domain suitable for incorporation in the proteins of the invention is encoded by the nucleic acid sequence set out in SEQ ID NO: 34.

Alternatively, a fragment or derivative of the sequence set out in SEQ ID NO: 34 may be used to encode a suitable ArgF enzyme domain for incorporation in a protein of the invention.

Any enzyme domain that confers ArgF activity on the proteins of the invention may be used in such an embodiment.

A suitable level of ArgF activity may be equivalent to that conferred by a protein encoded by SEQ ID NO: 34 Alternatively, enzyme domains conferring lower or higher levels of ArgF activity may be still of benefit.

### Tryptophan or a Tryptophan Precursor

The present invention relates to proteins that comprise a domain that promotes synthesis of tryptophan or a tryptophan precursor.

Tryptophan (frequently abbreviated to “Trp” or “W”) is a non-polar amino acid. It has a molecular mass of 204.2 g/mol-1, and may also be referred to as 2-Amino-3-(1H-indol-3-yl)propanoic acid.

For the purposes of the present invention, a “tryptophan precursor” may be taken as being any compound that precedes tryptophan in the tryptophan production cascade

A Domain that Promotes Synthesis of Tryptophan or a Tryptophan Precursor

The proteins of the invention comprise a domain that promotes synthesis of tryptophan or a tryptophan precursor. The ability of a domain to fulfil this function, that is to say to promote synthesis of tryptophan or a tryptophan precursor, may be investigated by any suitable means or assay.

The skilled person will appreciate that a suitable means or assay may be selected with reference to the compound, for example tryptophan or a tryptophan precursor, synthesis of which is to be promoted. Merely by way of example, suitable assays by which the ability of a domain to promote the requisite synthesis are described further in the Examples, in relation to the characterisation of exemplary cells of the invention.

Suitably the domain that promotes synthesis of tryptophan or a tryptophan precursor may be an enzyme domain capable of promoting the synthesis of tryptophan or a tryptophan precursor. In such an embodiment the enzyme domain may comprise the full length enzyme domain, or a fragment or a variant of such a domain, as long as the domain exhibits the requisite activity.

In a suitable embodiment, the tryptophan precursor, synthesis of which is promoted, is indoleglycerol phosphate. In such an embodiment, the domain that promotes such synthesis may be a tryptophan synthetase (TRP5) enzyme domain.

In a suitable embodiment, the synthesis of tryptophan is promoted. In such an embodiment, the domain that promotes such synthesis may be a indoleamine 2,3-dioxygenase (IDO) enzyme domain.

### An Tryptophan Synthetase (TRP5) Enzyme Domain

An example of an TRP5 enzyme domain suitable for incorporation in the proteins of the invention is encoded by the nucleic acid sequence set out in SEQ ID NO: 35.

Alternatively, a fragment or derivative of the sequence set out in SEQ ID NO: 35 may be used to encode a suitable TRP5 enzyme domain for incorporation in a protein of the invention.

Any enzyme domain that confers TRP5 activity on the proteins of the invention may be used in such an embodiment.

A suitable level of TRP5 activity may be equivalent to that conferred by a protein encoded by SEQ ID NO: 35 Alternatively, enzyme domains conferring lower or higher levels of TRP5 activity may be still of benefit.

### An Indoleamine 2,3-Dioxygenase (IDO) Enzyme Domain

An example of an IDO enzyme domain suitable for incorporation in the proteins of the invention is encoded by the nucleic acid sequence set out in SEQ ID NO: 36.

Alternatively, a fragment or derivative of the sequence set out in SEQ ID NO: 36 may be used to encode a suitable IDO enzyme domain for incorporation in a protein of the invention.

Any enzyme domain that confers IDO activity on the proteins of the invention may be used in such an embodiment.

A suitable level of IDO activity may be equivalent to that conferred by a protein encoded by SEQ ID NO: 36 Alternatively, enzyme domains conferring lower or higher levels of IDO activity may be still of benefit.

### Target Binding Moieties

The proteins of the invention comprise a target binding moiety. The target binding moiety confers specificity of binding of the proteins, and hence of the cytocidal activity of the cells expressing proteins of the invention, to target structures, such as cells, on which a target molecule, recognised by the target binding moiety, is found.

In particular, the target binding moieties confer specificity of the biological activities of the cells of the invention (for example, cytocidal activity, or cell proliferation in response to activation) that underpin their therapeutic utility. Except for where the context requires otherwise, references to specific binding in the present disclosure may be interpreted as referring to a target binding moiety's ability to discriminate between possible partners in the environment in which binding is to occur. A target binding moiety that interacts with one particular target molecule when other potential targets are present is said to “bind specifically” to the target molecule with which it interacts. In some embodiments, specific binding is assessed by detecting or determining degree of association between the target binding moiety and its target molecule; in some embodiments, specific binding is assessed by detecting or determining degree of dissociation of a binding moiety-target molecule complex; in some embodiments, specific binding is assessed by detecting or determining ability of the target binding moiety to compete an alternative interaction between its target molecule and another entity. In some embodiments, specific binding is assessed by performing such detections or determinations across a range of concentrations. In a suitable embodiment, specific binding is assessed by determining the difference in binding affinity between cognate and non-cognate targets. For example, a target binding moiety that is specific may have a binding affinity for a cognate target molecule that is about 3-fold, 4-fold, 5-fold, 6-fold, 7-fold, 8-fold, 9-fold, 10-fold or more than binding affinity for a non-cognate target.

In the context of the present disclosure, “specificity” is a measure of the ability of a particular target binding moiety to distinguish its target molecule binding partner from other potential binding partners.

A suitable target binding moiety may be directed to any desired target molecule. The target binding moiety may be directed to a target molecule expressed exclusively, or extensively, by a target against which it is desired to direct the cytocidal activity of a cell expressing a protein of the invention. For example, a target binding moiety may be directed to a target molecule associated with a disease. Suitably the target binding moiety may be directed to a target molecule associated with cancer, or with an infection.

In a suitable embodiment, the target binding moiety is selected from the group consisting of: a GD2 target binding moiety; a CD33 target binding moiety; a mesothelin target binding moiety; and an EGFRvIII target binding moiety.

Examples of such target binding moieties are set out in SEQ ID NOs: 3 to 6. It will be appreciated that fragments or variants (for example, differing from the exemplary sequence by 1, 2, 3, 4, 5, or more amino acid residues) may be used as alternative target binding moieties, as long as the fragment or variant retains the ability to bind the target molecule.

Without limitation, suitable target binding moieties may be selected from the group consisting of: antibodies, or fragments (such as scFvs) or derivatives thereof; TCRs, such as TCR α chains or TCR β chains; and aptamers.

### A GD2 Target Binding Moiety

A GD2 target binding moiety is a moiety capable of binding to disialoganglioside 2 (GD2), which may also be referred to as ganglioside GD2. A protein of the invention comprising a GD2 target binding moiety is suitable for use in circumstances in which it is desired to exert the cytocidal activity of a cell expressing a protein of the invention against a target comprising GD2 molecules, for example a cell expressing GD2.

GD2 is expressed by cancers of neuroectodermal origin, including neuroblastoma, osteosarcoma and melanoma. Therefore, it will be appreciated that a protein (such as a CAR) of the invention comprising a GD2 target binding moiety is suitable for use in circumstances in which it is desired to utilise a protein of the invention in a medical use for the prevention and/or treatment of any such cancers, and particularly neuroblastoma.

A GD2 target binding moiety suitable for incorporation in a protein in accordance with the invention may be an anti-GD2 antibody, such as an anti-GD2 monoclonal antibody, or an antigen binding fragment or derivative thereof. For example, a GD2 target binding moiety may be an anti-GD2 scFv antibody fragment. Merely by way of example, a suitable GD2 targeting domain comprising an scFv antibody fragment is set out in SEQ ID NO: 3.

The scFv antibody fragment set out in SEQ ID NO: 3 is also referred to as the 14g2a scFv, as described in U.S. Pat. No. 9,493,740 B2. It is derived from the ch14.18 antibody disclosed in U.S. Pat. No. 9,777,068 B2, and it will be appreciated that other ch14.18 antibody fragments or variants may be used as GD2 target binding moieties in the proteins of the invention.

Alternatively, a suitable GD2 target binding moiety may be selected from the group consisting of: an anti-GD2 aptamer; or a fragment or derivative thereof.

Suitably a GD2 target binding moiety is capable of binding specifically to GD2.

### A CD33 Target Binding Moiety

A CD33 target binding moiety is a moiety capable of binding to CD33 (also known as Siglec-3). CD33 is transmembrane protein. A protein of the invention comprising a CD33 target binding moiety is suitable for use in circumstances in which it is desired to exert the biological activity of a cell expressing a protein of the invention against a target comprising CD33 molecules, for example a cell expressing CD33.

CD33 is expressed by acute myeloid leukaemia (AML) cells. Therefore, it will be appreciated that a protein of the invention comprising a CD33 target binding moiety is suitable for use in circumstances in which it is desired to utilise a protein of the invention in a medical use for the prevention and/or treatment of AML.

A CD33 target binding moiety suitable for incorporation in a protein in accordance with the invention may be an anti-CD33 antibody, such as an anti-CD33 monoclonal antibody, or an antigen binding fragment or derivative thereof. For example, a CD33 target binding moiety may be an anti-CD33 scFv antibody fragment. Merely by way of example, a suitable CD33 targeting domain comprising an scFv antibody fragment is set out in SEQ ID NO: 4.

The scFv antibody fragment is set out in SEQ ID NO: 4 is derived from the humanised my96 clone monoclonal antibody. Details of the my96 antibody are set out in Leukemia. 2015 August; 29(8):1637-47, and details of the scFv fragment of SEQ ID NO: 4 are set out in US20160096892A1 (where this scFv is disclosed as SEQ ID NO: 147). It will be appreciated that other my96 antibody fragments or variants may be used as CD33 target binding moieties in the proteins of the invention.

Alternatively, a suitable CD33 target binding moiety may be selected from the group consisting of: an anti-CD33 aptamer; or a fragment or derivative thereof

Suitably a CD33 target binding moiety is capable of binding specifically to CD33.

### A Mesothelin Target Binding Moiety

A mesothelin target binding moiety is a moiety capable of binding to mesothelin. Mesothelin is a 40 kDa protein that is the product of the MSLN. A protein of the invention comprising a mesothelin target binding moiety is suitable for use in circumstances in which it is desired to exert the biological activity of a cell expressing a protein of the invention against a target comprising mesothelin molecules, for example a cell expressing mesothelin.

Mesothelin is expressed by cells of a number of different types of cancers. Mesothelin expressing cancers include, for example, epithelial cancers, such as ovarian cancer, lung adenocarcinoma, and pancreatic cancer. Therefore, it will be appreciated that a protein of the invention comprising a mesothelin target binding moiety is suitable for use in circumstances in which it is desired to utilise a protein of the invention in a medical use for the prevention and/or treatment of any mesothelin expressing cancer.

A mesothelin target binding moiety suitable for incorporation in a protein in accordance with the invention may be an anti-mesothelin antibody, such as an anti-mesothelin monoclonal antibody, or an antigen binding fragment or derivative thereof. For example, a mesothelin target binding moiety may be an anti-mesothelin scFv antibody fragment. Merely by way of example, a suitable mesothelin targeting domain comprising an scFv antibody fragment is set out in SEQ ID NO: 5.

The scFv antibody fragment is set out in SEQ ID NO: 5 is derived from the SS1 antibody. Details of this antibody, and an scFV derived therefrom, are set out in WO 2015/090230 A (where the amino acid sequence of murine SS1 scFv is provided in SEQ ID NO: 279). It will be appreciated that other SS1 antibody fragments or variants may be used as mesothelin target binding moieties in the proteins of the invention.

Alternatively, a suitable mesothelin target binding moiety may be selected from the group consisting of: an anti-mesothelin aptamer; or a fragment or derivative thereof.

Suitably a GD2 target binding moiety is capable of binding specifically to GD2.

An EGFRvIII target binding moiety

A EGFRvIII target binding moiety is a moiety capable of binding to epidermal growth factor receptor variant III (EGFRvIII). A protein of the invention comprising a EGFRvIII target binding moiety is suitable for use in circumstances in which it is desired to exert the biological activity of a cell expressing a protein of the invention against a target comprising EGFRvIII molecules, for example a cell expressing EGFRvIII.

EGFRvIII is expressed by a range of cancers of epithelial origin. Therefore, it will be appreciated that a protein of the invention comprising an EGFRvIII target binding moiety is suitable for use in circumstances in which it is desired to utilise a protein of the invention in a medical use for the prevention and/or treatment of cancers expressing EGFR, such as glioblastomas, and colorectal cancers. In particular, a protein of the invention comprising an EGFRvIII target binding moiety is suitable for use in the prevention and/or treatment of glioblastoma.

An EGFRvIII target binding moiety suitable for incorporation in a protein in accordance with the invention may be an anti-EGFRvIII antibody, such as an anti-EGFRvIII monoclonal antibody, or an antigen binding fragment or derivative thereof. For example, a EGFRvIII target binding moiety may be an anti-EGFRvIII scFv antibody fragment. Merely by way of example, a suitable EGFRvIII targeting domain comprising an scFv antibody fragment is set out in SEQ ID NO: 6.

The scFv antibody fragment is set out in SEQ ID NO: 6 is derived from the 139 antibody disclosed in WO 2012/138475 A1 (in which a human scFV of the 139 antibody is set out as SEQ ID NO: 5, and a CAR construct incorporating the scFv is set out as SEQ ID NO: 11). It will be appreciated that other 139 antibody fragments or variants may be used as mesothelin target binding moieties in the proteins of the invention.

An alternative EGFRvIII target binding moiety may be derived from the MR1 anti-EGFRvIII antibody. An example of such an EGFRvIII target binding moiety is the scFv (derived from MR1) encoded by the DNA sequence of SEQ ID NO: 41. This alternative EGFRvIII target binding moiety is incorporated in the exemplary proteins of the invention set out in SEQ ID NO: 42, SEQ ID NO: 43, and SEQ ID NO: 44. These exemplary proteins of the invention were utilised in the studies described in the Examples.

Alternatively, a suitable EGFRvIII target binding moiety may be selected from the group consisting of: an anti-EGFRvIII aptamer; or a fragment or derivative thereof.

Suitably a EGFRvIII target binding moiety is capable of binding specifically to EGFRvIII.

### Intracellular Signalling Regions

The proteins of the invention comprise at least one intracellular signalling region. The intracellular signalling region serves to couple binding of the target binding moiety to a target molecule with other biological activities of the cell expressing the protein. In particular, a suitable intracellular signalling region may couple binding of the target binding moiety to its target molecule with activation of the cell's cytocidal activity and/or to the cells ability to proliferate in response to activation.

As set out in the Examples, a suitable intracellular signalling region may activate cytotoxic or specific cytolytic activity in response to binding of the target molecule to the target binding moiety. Alternatively, or additionally, a suitable intracellular signalling region may facilitate activation-induced cell proliferation in response to binding of the target molecule to the target binding moiety.

In a suitable embodiment, the intracellular signalling region comprises a region selected from the group consisting of: a 4-1BB signalling region; an OX-40 signalling region; a CD28 signalling region; an ICOS signalling region; and a CD3ζ signalling region.

It will be appreciated that proteins in accordance with the invention may comprise a plurality of intracellular signalling regions. Suitably the plurality may comprise more than one copy of an individual intracellular signalling region. For example, a protein of the invention may comprise multiple copies of one, or more, of: a 4-1BB signalling region; an OX-40 signalling region; a CD28 signalling region; an ICOS signalling region; and a CD3ζ signalling region.

Additionally, or alternatively, a protein of the invention may comprise a combination of multiple intracellular signalling regions. For example, a protein in accordance with the invention may comprise a combination of intracellular signalling regions selected from the group consisting of: a 4-1BB signalling region; an OX-40 signalling region; a CD28 signalling region; an ICOS signalling region; and a CD3ζ signalling region. Merely by way of example, a protein of the invention may comprise both a 4-1BB signalling region and a CD3ζ signalling region.

A suitable a 4-1BB signalling region is one that is able to provide sufficient costimulatory signalling to a cell expressing a protein comprising such a signalling region to promote at least one of: activation of the cell, and/or function of the cell, such as cytokine release by the cell, and/or cytotoxicity by the cell; and/or proliferation and/or persistence of the cell. This persistence may be persistence of the in vivo or in vitro. The persistence may, in particular, be persistence of the cell in conditions of the immunosuppressive tumour microenvironment, or that replicate this microenvironment. By way of example, the cytokine release may include one or more cytokines from the group consisting of: IFN-gamma, and/or TNFα, and/or IL2.

Suitably the 4-1BB signalling region may comprise the full-length sequence of 4-1BB. Alternatively, a 4-1BB signalling region may comprise a truncated and/or modified form of the full-length sequence. Merely by way of example, a suitable 4-1BB signalling region may comprise the amino acid sequence set out in SEQ ID NO: 7, or a portion of this sequence. Suitably a 4-1BB signalling region for incorporation in a protein of the invention may consist of the amino acid sequence set out in SEQ ID NO: 7.

In a suitable embodiment, an OX-40 signalling region is one that is able to provide sufficient costimulatory signalling to a cell expressing a protein comprising such a signalling region to promote at least one of: activation of the cell, and/or function of the cell, and/or persistence of the cell. This persistence may be persistence of the in vivo or in vitro. The persistence may, in particular, be persistence of the cell in conditions of the immunosuppressive tumour microenvironment, or that replicate this microenvironment.

Suitably the OX-40 signalling region may comprise the full-length sequence of OX-40. Alternatively, an OX-40 signalling region may comprise a truncated and/or modified form of the full-length sequence. Merely by way of example, a suitable OX-40 signalling region may comprise the amino acid sequence set out in SEQ ID NO: 8, or a portion of this sequence. Suitably an 4-1 OX-40 BB signalling region for incorporation in a protein of the invention may consist of the amino acid sequence set out in SEQ ID NO: 8.

A suitable CD28 signalling region is one that is able to provide sufficient costimulatory signalling to a cell expressing a protein comprising such a signalling region to promote at least one of: activation of the cell, and/or function of the cell (, and/or persistence of the cell. This persistence may be persistence of the in vivo or in vitro. The persistence may, in particular, be persistence of the cell in conditions of the immunosuppressive tumour microenvironment, or that replicate this microenvironment.

Suitably the CD28 signalling region may comprise the full-length sequence of CD28. Alternatively, a CD28 signalling region may comprise a truncated and/or modified form of the full-length sequence. Merely by way of example, a suitable CD28 signalling region may comprise the amino acid sequence set out in SEQ ID NO: 9, or a portion of this sequence. Suitably a CD28 signalling region for incorporation in a protein of the invention may consist of the amino acid sequence set out in SEQ ID NO: 9.

An ICOS signalling region is one that is able to provide sufficient costimulatory signalling to a cell expressing a protein comprising such a signalling region to promote at least one of: activation of the cell, and/or function of the cell, such as cytokine release by the cell, and/or cytotoxicity by the cell; and/or proliferation and/or persistence of the cell. This persistence may be persistence of the in vivo or in vitro. The persistence may, in particular, be persistence of the cell in conditions of the immunosuppressive tumour microenvironment, or that replicate this microenvironment.

Suitably the ICOS signalling region may comprise the full-length sequence of ICOS (also known as CD278). Alternatively, an ICOS signalling region may comprise a truncated and/or modified form of the full-length sequence. Merely by way of example, a suitable ICOS signalling region may comprise the amino acid sequence set out in SEQ ID NO: 10, or a portion of this sequence. Suitably an ICOS signalling region for incorporation in a protein of the invention may consist of the amino acid sequence set out in SEQ ID NO: 10. A truncated or modified form of ICOS may comprise at least the YMFM motif found at residues 180-183 of the full-length ICOS protein.

A suitable CD3ζ signalling region is one that is able to activate a functional response within the T cell (e.g. cytokine release (e.g. interferon-gamma, TNFa and/or IL2), cytotoxicity and/or proliferation.)

Suitably the CD3ζ signalling region may comprise the full-length sequence of CD3 Alternatively, a CD3ζ signalling region may comprise a truncated and/or modified form of the full-length sequence. Merely by way of example, a suitable CD3ζ signalling region may comprise the amino acid sequence set out in SEQ ID NO: 11 or SEQ ID NO: 40, or a portion of these sequences. Suitably a CD3ζ signalling region for incorporation in a protein of the invention may consist of the amino acid sequences set out in SEQ ID NO: 11 or SEQ ID NO: 40.

### Proteins of the Invention Targeting GD2

A protein of the invention that targets GD2 may comprise a GD2 targeting moiety, in combination with a suitable intracellular signalling region (such as a 4-1BB intracellular signalling region and a CD3ζ intracellular signalling region). The protein may further comprise an ASS-1 domain; and/or an OTC domain; and/or an ASL domain; and/or an OCD1 domain; and/or an ArgG domain; and/or an ArgH domain; and/or an ArgF domain. Suitably, the protein may comprise an ASS-1 domain; and/or an OTC domain.

The inventors have found that proteins of the invention comprising a GD2 targeting moiety in combination with an ASS-1 domain and/or an OTC domain are particularly useful with respect to a number of properties that demonstrate their therapeutic utility.

For example, the inventors have found that cells expressing a protein of the invention comprising a GD2 targeting moiety in combination with an ASS-1 domain and/or an OTC domain exhibit viability that is comparable, or improved, as compared to GD2 CAR-T cells known in the prior art.

Advantageously, the inventors have found that cells expressing proteins of the invention comprising a GD2 targeting moiety in combination with an ASS-1 domain and/or an OTC domain are able to demonstrate increased persistence compared to comparable control CAR-T cells. Cells expressing proteins of the invention comprising a GD2 targeting moiety in combination with an ASS-1 domain demonstrate particularly advantageous increased persistence compared to control cells.

The inventors have also found that cells expressing proteins of the invention comprising a GD2 targeting moiety in combination with an ASS-1 domain and/or an OTC domain are able to demonstrate increased proliferation compared to control CAR-T cells in conditions representative of the tumour microenvironment (such as experimentally arginine-depleted conditions).

In particular, cells expressing proteins of the invention comprising a GD2 targeting moiety in combination with an OTC domain are able to demonstrate increased proliferation compared to control cells in conditions, such as arginine-depleted conditions, representative of the tumour microenvironment. Surprisingly, cells expressing proteins of the invention comprising a GD2 targeting moiety in combination with an ASS-1 and an OTC domain are able to demonstrate an even greater increase in proliferation compared to control cells in conditions, such as arginine-depleted conditions, representative of the tumour microenvironment.

As shown in the Examples, cells expressing proteins of the invention comprising a GD2 targeting moiety in combination with an OTC domain are able to demonstrate a 5-fold increase in proliferation compared to control cells in conditions of the tumour microenvironment such as arginine-depleted conditions.

Even more surprisingly, cells expressing proteins of the invention comprising a GD2 targeting moiety in combination with an ASS-1 domain and an OTC domain are able to demonstrate a 10-fold increase in proliferation compared to control cells in conditions of the tumour microenvironment such as arginine-depleted conditions.

The inventors have found that cells expressing proteins of the invention comprising a GD2 targeting moiety in combination with an ASS-1 domain and/or an OTC domain demonstrate cytocidal activity in respect of cancer cells. Merely by way of example, they have demonstrated that cells expressing proteins of the invention comprising a GD2 targeting moiety in combination with an ASS-1 domain or an OTC domain are able to demonstrate specific cytocidal activity that is comparable to GD2 CAR-T cells known in the prior art.

Advantageously, cells expressing proteins of the invention comprising a GD2 targeting moiety in in combination with an ASS-1 domain or an OTC domain are able to demonstrate cytocidal activity, in addition to increased persistence and proliferation, that improves the survival of recipients in an in vivo cancer model.

These advantages are discussed further in the Examples section of the specification.

The amino acid sequence of exemplary proteins of the invention that target GD2 are set out in SEQ ID NOs: 12 to 14. The present invention should be taken as encompassing not only these specific proteins, but also as encompassing variants of these proteins that share the biological activity (particularly the cytocidal activity and ability to promote proliferation in response to protein binding) of these exemplary proteins. Such variants may share at least 80% sequence identity with any of the proteins of SEQ ID NOs: 12 to 14. Suitably, such variants may share at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with any of the proteins of SEQ ID NOs: 12 to 14.

### Proteins of the Invention Targeting Mesothelin

A protein of the invention that targets mesothelin may comprise a mesothelin targeting moiety derived from the anti-mesothelin SS1 antibody, in combination with a suitable intracellular signalling region (such as a 4-1 BB intracellular signalling region and a CD3ζ intracellular signalling region). The protein may further comprise an ASS-1 domain; and/or an OTC domain; and/or an ASL domain; and/or an OCD1 domain; and/or an ArgG domain; and/or an ArgH domain; and/or an ArgF domain. Suitably, the protein may comprise an ASS-1 domain; and/or an OTC domain.

The inventors have found that proteins of the invention comprising a mesothelin targeting moiety in combination with an ASS-1 domain and/or an OTC domain are particularly useful with respect to a number of properties that demonstrate their therapeutic utility.

For example, the inventors have found that cells expressing a protein of the invention comprising a mesothelin targeting moiety in combination with an ASS-1 domain and/or an OTC domain exhibit viability that is comparable, or improved, as compared to mesothelin CAR-T cells known in the prior art.

Advantageously, the inventors have found that cells expressing proteins of the invention comprising a mesothelin targeting moiety in combination with an ASS-1 domain and/or an OTC domain are able to demonstrate increased proliferation compared to control CAR-T cells in conditions representative of the tumour microenvironment (such as experimentally arginine-depleted conditions).

Cells expressing proteins of the invention comprising a mesothelin targeting moiety in combination with an OTC domain demonstrate particularly increased proliferation compared to control cells in conditions, such as arginine-depleted conditions, representative of the tumour microenvironment. As illustrated in the Examples, this particular increase in proliferation is demonstrated by proteins of the invention comprising an OTC domain on its own, or with an ASS-1 domain. It can be seen that cells expressing proteins of the invention comprising a mesothelin targeting moiety in combination with an OTC domain demonstrate an approximately 4-fold increase in proliferation compared to control cells in conditions replicating those found in the tumour microenvironment.

Also shown in the Examples, cells expressing proteins of the invention comprising a mesothelin targeting moiety in combination with an ASS-1 domain and an OTC domain are able to demonstrate a more than a 3-fold increase in proliferation compared to control cells in conditions of the tumour microenvironment such as arginine-depleted conditions.

These advantages are discussed further in the Examples section of the specification.

The amino acid sequence of exemplary proteins of the invention that target mesothelin are set out in SEQ ID NOs: 15 to 17. The present invention should be taken as encompassing not only these specific proteins, but also as encompassing variants of these proteins that share the biological activity (particularly the cytocidal activity and ability to promote proliferation in response to protein binding) of these exemplary proteins. Such variants may share at least 80% sequence identity with any of the proteins of SEQ ID NOs: 15 to 17. Suitably, such variants may share at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with any of the proteins of SEQ ID NOs: 15 to 17.

### Proteins of the Invention Targeting CD33

A protein of the invention that targets CD33 may comprise a CD33 targeting moiety derived from the anti-CD33 my96 antibody, in combination with a suitable intracellular signalling region (such as a 4-1BB intracellular signalling region and a CD3 intracellular signalling region). The protein may further comprise an ASS-1 domain; and/or an OTC domain ASS-1 domain; and/or an OTC domain; and/or an ASL domain; and/or an OCD1 domain; and/or an ArgG domain; and/or an ArgH domain; and/or an ArgF domain. Suitably, the protein may comprise an ASS-1 domain; and/or an OTC domain.

Proteins of the invention comprising a CD33 targeting moiety in combination with an ASS-1 domain and/or an OTC domain are particularly useful with respect to a number of properties that demonstrate their therapeutic utility.

For example, the inventors have found that cells expressing a protein of the invention comprising a CD33 targeting moiety in combination with an ASS-1 domain and/or an OTC domain exhibit viability that is comparable, or improved, as compared to CD33 CAR-T cells known in the prior art.

Advantageously, the inventors have found that cells expressing proteins of the invention comprising a CD33 targeting moiety in combination with an ASS-1 domain and/or an OTC domain are able to demonstrate increased persistence compared to comparable control CAR-T cells. Cells expressing proteins of the invention comprising a CD33 targeting moiety in combination with an ASS-1 domain demonstrate particularly advantageous increased persistence compared to control cells.

The inventors have also found that cells expressing proteins of the invention comprising a CD33 targeting moiety in combination with an ASS-1 domain and/or an OTC domain are able to demonstrate increased proliferation compared to control CAR-T cells in conditions representative of the tumour microenvironment (such as experimentally arginine-depleted conditions).

Cells expressing proteins of the invention comprising a CD33 targeting moiety in combination with an OTC domain demonstrate significantly increased proliferation compared to control cells in conditions, such as arginine-depleted conditions, representative of the tumour microenvironment. The inventors have also found that cells expressing proteins of the invention comprising a CD33 targeting moiety in combination with an ASS-1 domain and an OTC domain demonstrate an even greater increase in proliferation compared to control cells in conditions representative of the tumour microenvironment.

As shown in the Examples, cells expressing proteins of the invention comprising a CD33 targeting moiety in combination with an OTC domain demonstrate a more than 5-fold increase in proliferation compared to control cells in conditions representing the tumour microenvironment. Even more beneficially, cells expressing proteins of the invention comprising a CD33 targeting moiety in combination with both an ASS-1 domain and an OTC domain demonstrate an approximately 6-fold increase in proliferation compared to control cells under the same conditions.

The inventors have found that cells expressing proteins of the invention comprising a CD33 targeting moiety in combination with an ASS-1 domain and/or an OTC domain demonstrate cytocidal activity in respect of cancer cells that is comparable to that of CD33 CAR-T cells known in the prior art.

Advantageously, cells expressing proteins of the invention comprising a CD33 targeting moiety in combination with an ASS-1 domain and/or an OTC domain are able to demonstrate cytocidal activity, in addition to increased persistence and proliferation, that improves the survival of recipients in an in vivo cancer model.

These advantages are discussed further in the Examples section of the specification.

The amino acid sequence of exemplary proteins of the invention that target CD33 are set out in SEQ ID NOs: 18 to 20. The present invention should be taken as encompassing not only these specific proteins, but also as encompassing variants of these proteins that share the biological activity (particularly the cytocidal activity and ability to promote proliferation in response to protein binding) of these exemplary proteins. Such variants may share at least 80% sequence identity with any of the proteins of SEQ ID NOs: 18 to 20. Suitably, such variants may share at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with any of the proteins of SEQ ID NOs: 18 to 20.

### Proteins of the Invention Targeting EGFRvIII

A protein of the invention that targets EGFRvIII may comprise an EGFRvIII targeting moiety derived from the anti-EGFRvIII 139 antibody, in combination with a suitable intracellular signalling region (such as a 4-1BB intracellular signalling region and a CD3 intracellular signalling region). The protein may further comprise an ASS-1 domain and/or an OTC domain; and/or an ASL domain; and/or an OCD1 domain; and/or an ArgG domain; and/or an ArgH domain; and/or an ArgF domain. In particular, the protein may comprise an ASS-1 domain, and/or an OTC domain.

The inventors' results demonstrate that cells expressing a protein of the invention comprising an EGFRvIII targeting moiety in combination with an ASS-1 domain and/or an OTC domain exhibit viability that is comparable, or improved, as compared to EGFRvIII CAR-T cells known in the prior art.

The inventors have found that cells expressing proteins of the invention comprising an EGFRvIII targeting moiety in combination with an ASS-1 domain demonstrate increased proliferation compared to control CAR-T cells in conditions representative of the tumour microenvironment (such as experimentally arginine-depleted conditions). Such cells expressing proteins of the invention comprising an EGFRvIII targeting moiety in combination with an ASS-1 domain demonstrate a more than 2-fold increase in proliferation compared to control cells in such conditions.

These advantages are discussed further in the Examples section of the specification.

The amino acid sequence of exemplary proteins of the invention that target EGFRvIII are set out in SEQ ID NOs: 21 to 23. The present invention should be taken as encompassing not only these specific proteins, but also as encompassing variants of these proteins that share the biological activity (particularly the cytocidal activity and ability to promote proliferation in response to protein binding) of these exemplary proteins. Such variants may share at least 80% sequence identity with any of the proteins of SEQ ID NOs: 21 to 23. Suitably, such variants may share at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with any of the proteins of SEQ ID NOs: 21 to 23.

### Nucleic Acids Encoding Proteins of the Invention

The third aspect of the invention provides a nucleic acid encoding a protein of the invention. The proteins may be in accordance with any of the aspects or embodiments of the invention described herein.

Suitably a nucleic acid in accordance with the invention comprises DNA. In a suitable embodiment, a nucleic acid of the invention comprises RNA. It will be appreciated that a suitable nucleic acid may essentially consist of DNA, may essentially consist of RNA, or may comprise a combination of DNA and RNA.

Examples of nucleic acids encoding proteins of the invention are set out in SEQ ID NOs: 37 to 39. These nucleic acid sequences are DNA molecules encoding exemplary proteins set out in the specification as follows:

| Nucleic acid of the | Encodes protein of the |
|---|---|
| invention set out in | invention set out in |
| SEQ ID NO | SEQ ID NO |
| 37 | 12 |
| 38 | 13 |
| 39 | 14 |

It will be appreciated that codon degeneracy means that there can be notable differences in the sequences of nucleic acids of the invention encoding a single given protein of the invention.

Merely by way of example, a suitable nucleic acid of the invention may share at least 70% sequence identity with one of the exemplary nucleic acids of the invention set out in SEQ ID NOs: 37 to 39. A suitable nucleic acid of the invention may share at least 75% sequence identity; at least 80% sequence identity; at least 85% sequence identity; at least 90% sequence identity; at least 95% sequence identity; at least 96% sequence identity; at least 97% sequence identity; at least 98% sequence identity; or even 99% or more sequence identity with one of the exemplary nucleic acids of the invention set out in SEQ ID NOs: 37 to 39.

A nucleic acid sequence encoding a protein of the invention that targets mesothelin may be the same as the nucleic acid sequences of any of SEQ ID NOs: 37, 38, or 39 save that the part of those nucleic acid sequences that encodes the target binding moiety is replaced with the nucleic acid sequence of SEQ ID NO: 28.

A nucleic acid sequence encoding a protein of the invention that targets CD33 may be the same as the nucleic acid sequences of any of SEQ ID NOs: 37, 38, or 39 save that the part of those nucleic acid sequences that encodes the target binding moiety is replaced with the nucleic acid sequence of SEQ ID NO: 27.

A nucleic acid sequence encoding a protein of the invention that targets EGFRvII may be the same as the nucleic acid sequences of any of SEQ ID NOs: 37, 38, or 39 save that the part of those nucleic acid sequence that encodes the target binding moiety is replaced with the nucleic acid sequence of SEQ ID NO: 29.

A nucleic acid encoding a protein of the invention may be provided in the form of a vector. Suitably the vector may be a viral vector, such as a retroviral vector or a lentiviral vector, or a transposon. Both retroviral and lentiviral approaches have been used successfully in the production of cells of the invention.

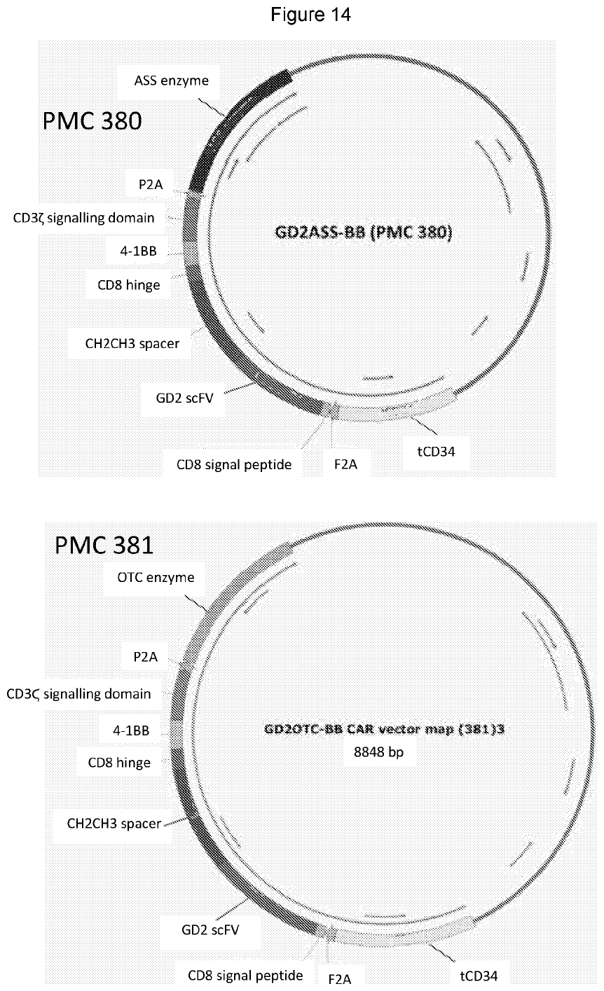

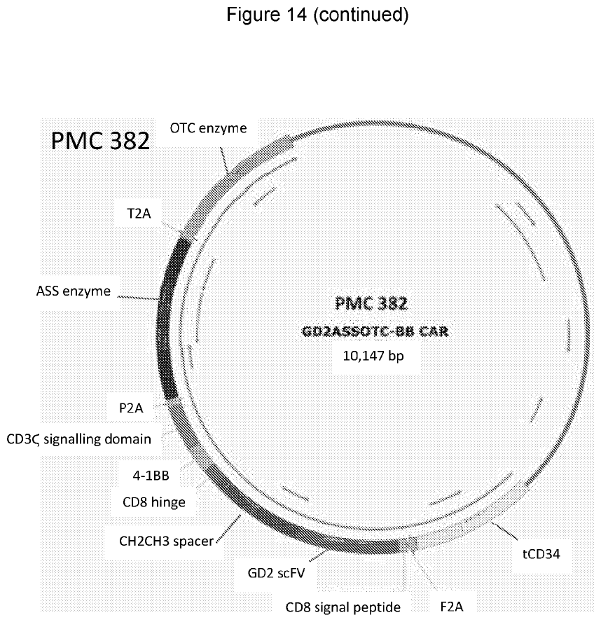

Details of constructs that have been used in the successful lentiviral production of cells of the invention are set out in FIG. 14. These constructs provide further examples of nucleic acids of the invention.

### Cells of the Invention

The second aspect of the invention provides a cell comprising a protein in accordance with the first aspect of the invention. The cell may express the protein. The protein may be in accordance with any of the embodiments of the first aspect of the invention described herein.

Suitably a cell in accordance with the second aspect of the invention may be a cell is a cell able to exert a cell-mediated immune response. A suitable cell may be able to exert cytocidal activity, for example by cytotoxic action, or by inducing specific cell lysis. Additionally, a suitable cell may be able to proliferate in response to binding of the protein to its corresponding target molecule. Suitably, a cell in accordance with the second aspect of the invention may be selected from the group consisting of: a T cell; and a natural killer (NK) cell.

Suitably a T cell may be selected from the group consisting of: an invariant natural killer T cell (iNKT); a natural killer T cell (NKT); a gamma delta T cell (gd T cell); an alpha beta T cell (ab T cell); an effector T cell; and a memory T cell.

Suitably a T cell may be selected from the group consisting of: a CD4⁺ lymphocyte; and a CD8⁺ lymphocyte.

The cell may be from a subject requiring prevention and/or treatment of a disease. The cell may be taken from a sample from such a subject.

Alternatively, the cell may be from a healthy donor subject (for the purposes of the present disclosure taken as a subject not afflicted with the disease to be treated with the protein or cell of the invention).

It will be appreciated that suitable cells may also include cells of cell lines.

Standard techniques for the collection of human cells, and their transformation with proteins such as the proteins of the invention, are well known to those skilled in the art. Preferred techniques for the retroviral transduction of human T cells, determination of transduction efficiency, and sorting of transduced T cells by magnetic activated cell sorting, are described further in the Examples.

### Biological Activity of Cells of the Invention

Cells of the invention, comprising proteins of the invention exhibit a number of activities that are of benefit in applications such as the prevention and/or treatment of diseases.

These biological activities may be further considered with reference to cytocidal activities (which represent the means by which the cells of the invention are able to exert their therapeutic effects) and activities such as proliferation (for example in response to activation) and persistence in vivo, which enable the cells of the invention to exert their therapeutic effects for longer than has been the case for CAR-expressing cells of the prior art.

These respective biological activities are described further below. It will be appreciated that the advantages offered by the proteins and cells of the invention arise primarily as a result of the combination of these biological activities.

Biological activity of the cells of the invention may be determined with reference to suitable comparator cells. Examples of suitable comparator cells include cells of the same type as those of the invention that have not been transduced with a protein, or cells of the same type as those of the invention that have been transduced with a protein that does not comprise a domain that promotes synthesis of arginine or an arginine precursor.

### Cytocidal Activity of Cells of the Invention

For the purposes of the present invention, cytocidal activity should be taken as encompassing any activity by which cells of the invention (for example cells expressing proteins of the invention) kill other cells. By way of example, the killing of other cells may be achieved by means of cytotoxic action of the cells of the invention, or by specific cell lysis mediated by the cells of the invention.

The cells of the invention exert their cytocidal activity in respect of target structures that comprise target molecules bound by the target binding moieties of the proteins of the invention.

Preferably the cells killed by cytocidal activity of cells of the invention are cells associated with a disease. Suitably the cells associated with a disease may be cancer cells, or infected cells.

As set out in the Examples, the inventors have demonstrated that cells of the invention (comprising proteins of the invention) exhibit cytocidal activity that is specifically directed to cells expressing target molecules bound by the target binding moieties of the proteins of the invention. The extent of cytocidal activity observed in respect of the cells of the invention is broadly in line with that of protein expressing cells described in the prior art. However, the combination of this maintained cytocidal activity, with improved proliferation and/or persistence, exhibited by the cells of the invention confers benefits not noted in respect of the cells of the prior art.

The skilled person will be aware of many suitable assays by which the cytocidal activity, whether cytotoxic activity or specific cell lysis, of a cell of the invention, or suitable comparator cell, may be assessed. Merely by way of example suitable assays are described in the Examples, where they are used in the characterisation of exemplary cells of the invention.

The skilled reader, on considering the information set out in the Examples, will recognise that the cells of the invention exhibit cytocidal activity that makes them well suited to therapeutic use in the prevention and/or treatment of disease in the manner described in this specification.

### Persistence of Cells of the Invention

Persistence in vivo, and particularly within a subject, of cells exerting a therapeutic effect is important for effective prevention and/or treatment of diseases. As mentioned previously, the microenvironment around tumours, such as neuroblastoma, is particularly damaging to therapeutic cells, such as CAR T cells. The effects of this microenvironment, and the inability of therapeutic cells to persist within it, is believed to have contributed significantly to the failures observed in respect of many prior art treatments.

Cells of the invention, comprising proteins of the invention, exhibit increased persistence in the tumour microenvironment. This increased persistence in vivo, which is demonstrated in the Examples, represents a mechanism by which the therapeutic effects of the cells of the invention can be prolonged, and so their therapeutic utility increased, as compared to prior art cells.

Persistence of cells of the invention, or suitable comparator cells, may be assessed experimentally in a number of different ways. Merely by way of example cell persistence may be defined with reference to the percentage of cells originally administered that remain viable within a recipient after a given period of time. It will be appreciated that a useful comparison between two or more populations of cells (such as a population of cells of the invention and a population of suitable comparator cells) may be made after any given period of time, so long as the time elapsed is approximately the same for each of the populations of cells. That said, the inventors have found that comparisons made 17 days after administration of cells are well suited to such calculations, for example in the case of NOG-SCID mice engrafted with 5×10⁶ of the cells of the invention, as shown in the Examples. It will be appreciated that other timepoints may be utilised with reference to particular experimental models of interest, and that other methods of measuring persistence in cells will be known to those skilled in the art.

The proportion of cells of the invention persisting after a set period of time may be at least 5% higher than that of suitable comparator cells. Indeed, the proportion of cells of the invention persisting may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% higher than that of suitable comparator cells. The proportion of cells of the invention persisting after a set period of time may be at least 100%, or more, higher than that of suitable comparator cells.

The proportion of cells of the invention persisting after a set period of time may be up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, or even up to 100% of the total number of cells of the invention originally administered.

Cells of the invention may persist in a recipient for a longer period than do suitable comparator cells. Cells of the invention may persist in the recipient for up to 5% longer than a suitable comparator cell. Cells of the invention may persist in the recipient for up to 10% longer, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, or even up to 100% longer than a suitable comparator cell.

Another way in which persistence of cells, such as cells of the invention, may be assessed is with reference to the length of time a cell remains viable in a recipient. Suitably a cell of the invention, comprising a protein of the invention, may remain viable for at least 5 days, at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 90 days, at least 95 days, or at least 100 days or more in a recipient. Suitably, a cell of the invention, comprising a protein of the invention, may remain viable for at least 150 days, at least 200 days, at least 250 days, at least 300 days, or at least 350 days or more in a recipient. Suitably a cell of the invention comprising a protein of the invention, may remain viable for at least 6 months, at least 9 months, at least 12 months at least 15 months, at least 18 months, at least 21 months, at least 24 months or more in the recipient. Suitably a cell of the invention comprising a protein of the invention, may remain viable for at least 1 year, at least 2 years at least 3 years, at least 4 years, at least 5 years, at least 6 years, at least 7 years, at least 8 years, at least 9 years or at least 10 years or more in the recipient. Suitable, the cell of the invention comprising the protein of the invention, may remain viable for at least 10 years, for at least 15 years, for at least 20 years, for at least 25 years, for at least 30 years, for at least 35 years, for at least 40 years, for at least 45 years, for at least 50 years or more in the recipient.

### Proliferation of Cells of the Invention

Activation of cells of the invention, via binding of the protein to the corresponding target molecule, induces cell proliferation. This allows the production of increased numbers of cells able to exert a therapeutic activity. However, such cell proliferation is normally inhibited in the tumour microenvironment, and this has contributed to the failure of CAR T cell treatments disclosed in the prior art.

The cells of the invention exhibit proliferation capacity within the arginine-depleted tumour microenvironment that is remarkably improved as compared to that observed in respect of CAR T cells of the prior art. Since cell proliferation results in expansion of populations of cells of the invention that are then able to exert their therapeutic cytocidal activity within the tumour microenvironment, this is highly advantageous.

Proliferation of cells, such as cells of the invention may be assessed experimentally in a number of ways. Merely by way of example, cell proliferation may be assessed in conditions that replicate the tumour microenvironment. Such conditions may involve the use of culture media that have been depleted with respect to arginine, for example by conditioning with tumour cells. In such conditions, the cells of the invention may exhibit a rate of proliferation that is at least 5% higher than that of suitable comparator cells, at least 10% higher than that of suitable comparator cells, at least 15% higher than that of suitable comparator cells, at least 20% higher than that of suitable comparator cells, at least 25% higher than that of suitable comparator cells, at least 30% higher than that of suitable comparator cells, at least 35% higher than that of suitable comparator cells, at least 40% higher than that of suitable comparator cells, at least 45% higher than that of suitable comparator cells, at least 50% higher than that of suitable comparator cells, at least 55% higher than that of suitable comparator cells, at least 60% higher than that of suitable comparator cells, at least 65% higher than that of suitable comparator cells, at least 70% higher than that of suitable comparator cells, at least 75% higher than that of suitable comparator cells, at least 80% higher than that of suitable comparator cells, at least 85% higher than that of suitable comparator cells, at least 90% higher than that of suitable comparator cells, or at least 95% higher than that of suitable comparator cells. Indeed, the cells of the invention may exhibit a rate of proliferation that is at least 100%, or more, higher than that of suitable comparator cells in the same experimental conditions.

Alternatively, proliferation of cells may be assessed with reference to the number of cells present in a recipient after a set period of time from administration, as compared to the number of comparator cells present under the same conditions. The number of cells of the invention present in a recipient after a given time may be at least 5% higher than that of suitable comparator cells, at least 10% higher than that of suitable comparator cells, at least 15% higher than that of suitable comparator cells, at least 20% higher than that of suitable comparator cells, at least 25% higher than that of suitable comparator cells, at least 30% higher than that of suitable comparator cells, at least 35% higher than that of suitable comparator cells, at least 40% higher than that of suitable comparator cells, at least 45% higher than that of suitable comparator cells, at least 50% higher than that of suitable comparator cells, at least 55% higher than that of suitable comparator cells, at least 60% higher than that of suitable comparator cells, at least 65% higher than that of suitable comparator cells, at least 70% higher than that of suitable comparator cells, at least 75% higher than that of suitable comparator cells, at least 80% higher than that of suitable comparator cells, at least 85% higher than that of suitable comparator cells, at least 90% higher than that of suitable comparator cells, or at least 95% higher than that of suitable comparator cells if both cells of the invention and comparator cells are administered in approximately equal amounts.

### Medical Uses and Methods of Treatment

The proteins of the invention, particularly in the form of cells of the second aspect of the invention that express such proteins, are well suited to medical use, which is to say for use as medicaments in the prevention and/or treatment of diseases. Such medical uses are the subject matter of the fifth, sixth, and seventh aspect of the invention.

Prevention of a disease may be required when a subject has not yet developed a disease, but has been identified as being at risk of developing the disease in future. Suitably such identification may be based upon details such as the clinical history of the subject or their family, results of genetic testing of the subject of their family, or exposure risk to known disease causing agents. In the case of cancer, prevention may be desirable in the case of a subject exhibiting symptoms or features of pre-malignant disease.

Treatment of a disease may be required once a subject has been identified as already having developed a disease. The stage of development of the disease at the time of identification may be symptomatic or asymptomatic. Such identification may be based upon clinical assessment of the subject, symptoms presented by the subject, or analysis of samples provided by the subject (such biopsies, blood samples, or the like, allowing for the identification of the presence of malignancies, infectious agents, or other indicators of pathology).

The eighth aspect of the invention relates to a method of preventing and/or treating a disease in a subject in need of such prevention and/or treatment, the method comprising providing the subject with a protein of the invention. The protein of the invention is provided in a therapeutically effective amount. Such a therapeutically effective amount may be achieved by a single incidence of providing a protein of the invention, or cumulatively through multiple incidences of providing proteins of the invention.

The protein of the invention may suitably be provided to the subject directly or indirectly. By direct provision is meant the administration of the protein, particularly in the form of a cell expressing the protein, to the subject. By indirect provision is meant inducing the subject to express a protein of the invention. It will be appreciated that a protein of the invention may be provided indirectly to a subject via administration of a nucleic acid of the third aspect of the invention, which encodes a protein according to the first aspect of the invention.

It will be appreciated that cells expressing proteins may be used in the prevention or treatment of a wide range of diseases, including cancers, autoimmune diseases and diseases caused by infections, such as viral infections. Suitably such diseases may be prevented and/or treated by medical uses of methods of treatment utilising the proteins, cells, nucleic acids, or pharmaceutical compositions of the invention.

Prevention and/or Treatment of Cancer

In particular, the proteins, cells, nucleic acids, or pharmaceutical compositions of the invention may be of use in the prevention and/or treatment of cancer. It is in these applications that the ability of the cells of the invention to function, persist and proliferate in the arginine-depleted tumour microenvironment are particularly advantageous.

Suitable examples of cancers that may be prevented and/or treated by medical uses of methods of treatment utilising the proteins, cells, nucleic acids, or pharmaceutical compositions of the invention include those selected from the group consisting of: neuroblastoma; mesothelioma; ovarian cancer; breast cancer; colon cancer; medulloblastoma; pancreatic cancer; prostate cancer; testicular cancer; acute myeloid leukaemia; glioblastoma; osteosarcoma; and melanoma.

### Pharmaceutical Compositions and Formulation

The present invention also provides compositions including proteins, cells, or nucleic acids of the invention. In particular, the invention provides pharmaceutical compositions and formulations, such as unit dose form compositions including proteins, cells, or nucleic acids of the invention for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered,

A “pharmaceutically acceptable” carrier refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the choice of carrier is determined in part by the particular protein, cell, or nucleic acid of the invention, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001 to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatine, or immunoglobulins; hydrophilic polymers such as polyvinylpyrrolidone; amino acids such as glycine, glutamine, asparagine, histidine, arginine, or lysine; monosaccharides, disaccharides; and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents are included in some embodiments of the compositions of the invention. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001 to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the proteins s, cells, or nucleic acids of the invention, preferably those with activities complementary to the proteins s; cells; or nucleic acids of the invention, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g.; asparaginase; busulfan; carboplatin, cisplatin, daunorubicin, doxorubicin, fluorouracil; gemcitabine, hydroxyurea, methotrexate, paclitaxel, rituximab, vinblastine, and/or vincristine.

The pharmaceutical composition in some embodiments contains the CARs, cells, or nucleic acids of the invention in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the proteins, cells, or nucleic acids of the invention, by multiple bolus administrations of the proteins, cells, or nucleic acids, or by continuous infusion administration of the proteins, cells, or nucleic acids.

The compositions may be administered using standard administration techniques, formulations, and/or devices. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the proteins, cells, or nucleic acids of the invention in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavouring agents, and/or colours, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminium monostearate and gelatine.

The formulations to be used for in vivo administration are generally sterile. Sterility may be readily accomplished, e.g., by filtration through sterile filtration membranes.

### Doses and Dosage Regimens

### Size or Amount of Doses

The proteins, cells, or nucleic acids of the invention may be provided in a first dose, and optionally in subsequent doses. In some embodiments, the first or subsequent dose contains a number of proteins, cells, or nucleic acids of the invention in the range from about 10⁵ to about 10⁶ of such cells per kilogram body weight of the subject, and/or a number of such cells that is no more than about 10⁵ or about 10⁶ such cells per kilogram body weight of the subject. For example, in some embodiments, the first or subsequent dose includes less than or no more than at or about 1×10⁵, at or about 2×10⁵, at or about 5×10⁵, or at or about 1×10⁶ of such cells per kilogram body weight of the subject. In some embodiments, the first dose includes at or about 1×10⁵, at or about 2×10⁵, at or about 5×10⁵, or at or about 1×10⁶ of such cells per kilogram body weight of the subject, or a value within the range between any two of the foregoing values.

In some embodiments, for example, where the subject is a human, the first or subsequent dose includes fewer than about 1×10 total proteins, cells, or nucleic acids of the invention e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the first or subsequent dose contains fewer than about 1×10⁸ total proteins, cells, or nucleic acids of the invention per m² of the subject, e.g., in the range of about 1×10⁶ to 1×10 8 such cells per m 2 of the subject, such as 2×106, 5×106, 1×107, 5×107, or 1×108 such cells per m of the subject, or the range between any two of the foregoing values.

In certain embodiments, the number of proteins, cells, or nucleic acids of the invention in the first or subsequent dose is greater than about 1×10⁶ such proteins, cells, or nucleic acids of the invention per kilogram body weight of the subject, e.g., 2×10⁶, 3×10⁶, 5×10⁶, 1×10⁷, 5×10⁷, 1×10⁸, 1×10⁹, or 1×10¹⁰ such cells per kilogram of body weight and/or, I×108, or I×109, I×1010 such cells per m 2 of the subject or total, or the range between any two of the foregoing values.

In some embodiments, the number of proteins, cells, or nucleic acids of the invention administered in the subsequent dose is the same as or similar to the number of proteins, cells, or nucleic acids of the invention administered in the first dose in any of the embodiments herein, such as less than or no more than at or about 1×10⁵, at or about 2×10⁵, at or about 5×10⁵, or at or about 1×10⁶ of such cells per kilogram body weight of the subject. In some embodiments, the subsequent dose(s) contains at or about 1×10⁵, at or about 2×10⁵, at or about 5×10⁵, or at or about 1×10⁶ of such cells per kilogram body weight of the subject, or a value within the range between any two of the foregoing values. In some embodiments, such values refer to numbers of proteins, cells, or nucleic acids of the invention. In some aspects, the subsequent dose is larger than the first dose. For example, in some embodiments, the subsequent dose contains more than about 1×10⁶ proteins, cells, or nucleic acids of the invention per kilogram body weight of the subject, such as about 3×10⁶, 5×10⁶, 1×107, 1×108, or 1×109 such cells per kilogram body weight of the subject. In some embodiments, the amount or size of the subsequent dose is sufficient to reduce disease burden or an indicator thereof, and/or one or more symptoms of the disease or condition. In some embodiments, the second (or other subsequent) dose is of a size effective to improve survival of the subject, for example, to induce survival, relapse-free survival, or event-free survival of the subject for at least 6 months, or at least 1, 2, 3, 4, or 5 years. In some embodiments, the number of proteins, cells, or nucleic acids of the invention administered and/or number of such cells administered per body weight of the subject in the subsequent dose is at least 2-fold, 5-fold, 10-fold, 50-fold, or 100-fold or more greater than the number administered in the first dose. In some embodiments, disease burden, tumor size, tumor volume, tumor mass, and/or tumor load or bulk is reduced following the subsequent dose by at least at or about 50, 60, 70, 80, 90% or more compared to that immediately prior to the administration of the first dose or of the second (or other subsequent) dose.

In other embodiments, the number of proteins, cells, or nucleic acids of the invention administered in the subsequent dose is lower than the number of proteins, cells, or nucleic acids of the invention administered in the first dose.

In some embodiments, multiple subsequent doses are administered following the first dose, such that an additional dose or doses are administered following administration of the second (or other subsequent) dose. In some aspects, the number of cells administered to the subject in the additional subsequent dose or doses (i.e., the third, fourth, fifth, and so forth) is the same as or similar to the first dose, the second dose, and/or other subsequent dose. In some embodiments, the additional dose or doses are larger than prior doses.

In some aspects, the size of the first and/or subsequent dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the size of the first and/or subsequent dose is determined by the burden of the disease or condition in the subject. For example, in some aspects, the number of proteins, cells, or nucleic acids of the invention administered in the first dose is determined based on the tumour burden that is present in the subject immediately prior to administration of the first dose. In some embodiments, the size of the first and/or subsequent dose is inversely correlated with disease burden. In some aspects, as in the context of a large disease burden, the subject is administered a low number of proteins, cells, or nucleic acids of the invention, for example less than about 1×10⁶ proteins, cells, or nucleic acids of the invention per kilogram of body weight of the subject. In other embodiments, as in the context of a lower disease burden, the subject is administered a larger number of proteins, cells, or nucleic acids of the invention, such as more than about 1×10⁶ proteins, cells, or nucleic acids of the invention per kilogram body weight of the subject.

In some aspects, the number of proteins, cells, or nucleic acids of the invention administered in the subsequent dose is determined based on the tumour burden that is present in the subject following administration of the first dose. In some embodiments, e.g. where the first dose has decreased disease burden or has done so below a particular threshold amount or level, e.g., one above which there is an increased risk of toxic outcome, the subsequent dose is large, e.g. more than 1×10⁶ proteins s, cells, or nucleic acids of the invention per kilogram body weight, and/or is larger than the first dose. In other aspects, the number of proteins, cells, or nucleic acids of the invention administered in the subsequent dose is low, e.g. less than about 1×10⁶, e.g. the same as or lower than the first dose, where the first dose has reduced tumour burden to a small extent or where the first dose has not led to a detectable reduction in tumour burden.

In some embodiments, the number of proteins, cells, or nucleic acids of the invention administered in the first dose is lower than the number of proteins, cells, or nucleic acids of the invention administered in other methods, such as those in which a large single dose of cells is administered, such as to administer the proteins, cells, or nucleic acids of the invention in before an immune response develops. Thus, in some embodiments, the methods reduce toxicity or toxic outcomes as compared to other methods that involve administration of a larger dose.

In some embodiments, the first dose includes the proteins, cells, or nucleic acids of the invention in an amount that does not cause or reduces the likelihood of toxicity or toxic outcomes, such as cytokine release syndrome (CRS), severe CRS (sCRS), macrophage activation syndrome, tumour lysis syndrome, fever of at least at or about 38 degrees Celsius for three or more days and a plasma level of CRP of at least at or about 20 mg/dL, and/or neurotoxicity. In some aspects, the number of cells administered in the first dose is determined based on the likelihood that the subject will exhibit toxicity or toxic outcomes, such as CRS, sCRS, and/or CRS-related outcomes following administration of the cells. For example, in some embodiments, the likelihood for the development of toxic outcomes in a subject is predicted based on tumour burden. In some embodiments, the methods include detecting or assessing the toxic outcome and/or disease burden prior to the administration of the dose.

In some embodiments, the second (or other subsequent) dose is administered at a time point at which a clinical risk for developing cytokine-release syndrome (CRS), macrophage activation syndrome, or tumour lysis syndrome, or neurotoxicity is not present or has passed or has subsided following the first administration, such as after a critical window after which such events generally have subsided and/or are less likely to occur, e.g., in 60, 70, 80, 90, or 95% of subjects with a particular disease or condition.

### Timing of Doses

In some aspects, the timing of the second or subsequent dose is measured from the initiation of the first dose to the initiation of the subsequent dose. In other embodiments, the timing of the subsequent dose is measured from the completion of the first dose, or from the median day of administration of the first dose, e.g. in the context of split dosing, described herein, where a dose is administered over more than one day, e.g. over 2 days or over 3 days.

In some embodiments, whether a subsequent dose of proteins, cells, or nucleic acids of the invention distinct from that of the first dose is administered, is determined based on the presence or degree of an immune response or detectable immune response in the subject to the proteins, cells, or nucleic acids of the invention of the first dose. In some aspects, a subsequent dose containing cells expressing a different receptor than the cells of the first dose will be administered to a subject with a detectable host adaptive immune response, or an immune response that has become established or reached a certain level, stage, or degree.

In some embodiments, the second (or other subsequent) dose is administered at a point in time at which a second administration of proteins, cells, or nucleic acids of the invention is likely to be or is predicted to be eliminated by the host immune system. The likeliness of developing an immune response may be determined by measuring receptor-specific immune responses in the subject following administration of the first dose, as described herein.

For example, in some embodiments, subjects may be tested following the first (or other prior) dose and prior to the second (or other subsequent) dose to determine whether an immune response is detectable in the subject after the first dose. In some such embodiments, the detection of an immune response to the first dose may trigger the need to administer the second dose.

In some aspects, samples from the subjects may be tested to determine if there is a decline in or lower than desired exposure, for example, less than a certain number or concentration of cells, as described herein, in the subject after the first or prior dose. In some such aspects, the detection of a decline in the exposure of the subject to the cells may trigger the need to administer the second dose.

In some embodiments, the subsequent dose is administered at a point in time at which the disease or condition in the subject has not relapsed following the reduction in disease burden in response to the first or prior dose. In some embodiments, the disease burden reduction is indicated by a reduction in one or more factors, such as load or number of disease cells in the subject or fluid or organ or tissue thereof, the mass or volume of a tumour, or the degree or extent of metastases. Such a factor is deemed to have relapsed if after reduction in the factor in response to an initial treatment or administration, the factor subsequently increases.

In some embodiments, the second dose is administered at a point in time at which the disease has relapsed. In some embodiments, the relapse is in one or one or more factors, or in the disease burden generally. In some aspects, the subsequent dose is administered at a point in time at which the subject, disease burden, or factor thereof has relapsed as compared to the lowest point measured or reached following the first or prior administration, but still is lower compared to the time immediately prior to the first dose. In some embodiments, the subject is administered the subsequent dose at a point in time at which disease burden or factor indicative thereof has not changed, e.g. at a time when an increase in disease burden has been prevented.

In some embodiments, the subsequent dose is administered at a time when a host adaptive immune response is detected, has become established, or has reached a certain level, degree, or stage. In some aspects, the subsequent dose is administered following the development of a memory immune response in the subject.

In some aspects, the time between the administration of the first dose and the administration of the subsequent dose is about 28 to about 35 days, about 29 to about 35 days, or more than about 35 days. In some embodiments, the administration of the second dose is at a time point more than about 28 days after the administration of the first dose. In some aspects, the time between the first and subsequent dose is about 28 days.

In some embodiments, an additional dose or doses, e.g. subsequent doses, are administered following administration of the second dose. In some aspects, the additional dose or doses are administered at least about 28 days following administration of a prior dose. In some embodiments, no dose is administered less than about 28 days following the prior dose.

In some embodiments, e.g. where one or more consecutive doses are administered to the subject, the consecutive doses may be separated by about 7, about 14, about 15, about 21, about 27, or about 28 days. In some aspects, the consecutive dose is administered 21 days following a prior dose. In some embodiments, the consecutive dose is administered between 14 and 28 days following administration of a prior dose.

In any of the embodiments, the methods in some cases include the administration of the first or prior dose and the subsequent dose(s), and in other cases include the administration of the subsequent dose(s) to a subject who has previously received the first or prior dose but do not include the administration of the first or prior dose itself. Thus, the methods in some cases involve the administration of consolidating treatment, such as by administering a consolidating subsequent dose to a subject that has previously received a dose of proteins, cells, or nucleic acids of the invention.

In some embodiments, disease burden, tumor size, tumor volume, tumor mass, and/or tumor load or bulk is reduced following the subsequent dose by at least at or about 50, 60, 70, 80, 90% or more compared to that immediately prior to the administration of the first or prior dose or of the second or subsequent dose.

### Production of Cells Expressing Proteins of the Invention

The skilled person will be aware of suitable methods by which nucleic acids, such as the nucleic acids of the invention, may be used in the production of transduced cells expressing proteins. Such methods may be used in the production of cells of the invention, which express proteins of the invention.

Merely by way of example, suitable protocols that may be used in the production of cells of the invention are described further in the Examples below.

Other examples of methods for the production of cells expressing proteins of the invention will be apparent to those skilled in the art. Without limitation, these include methods by which nucleic acids of the invention are introduced to cells by means such as viruses or nanoparticles.

### EXAMPLES

The proteins of the invention were investigated with reference to exemplary CARs, as discussed further below.

### 1 Optimisation of CAR-Containing Viral Titres.

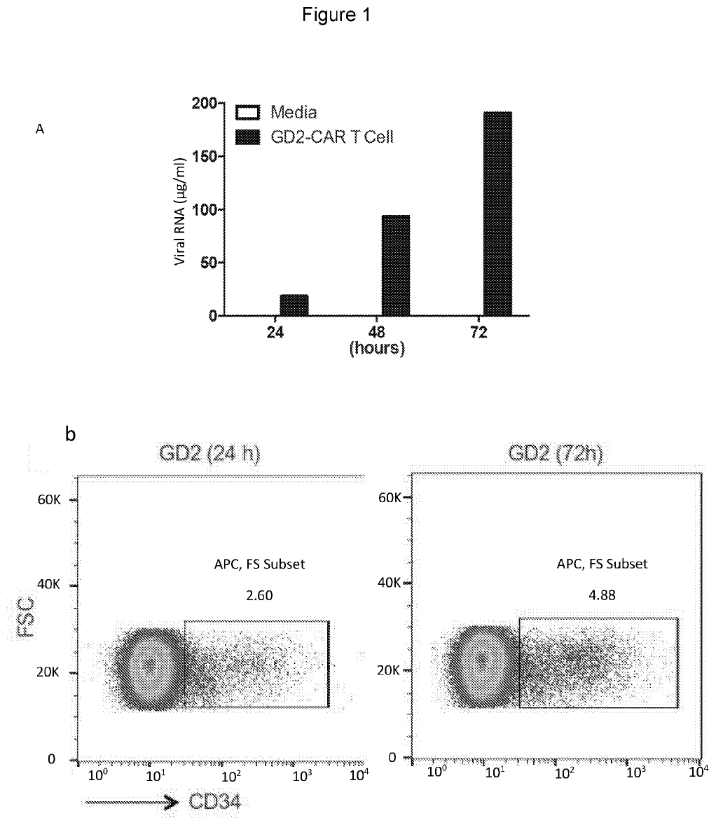

The inventors have shown that concentrations of retroviral particles increase over 72 hours in the supernatants of AMPHO Phoenix cells, shown in FIG. 1a.

CAR-T cell transduction efficiency was assessed by flow cytometry detection of tCD34. No difference in transduction efficiency of PBMCs was seen using AMPHO cell line supernatants collected at 24 or 72 hours, shown in FIG. 1b.

### 2 Arginine Pathway Enzymes Demonstrate Activity in Transduced Jurkat Cells.

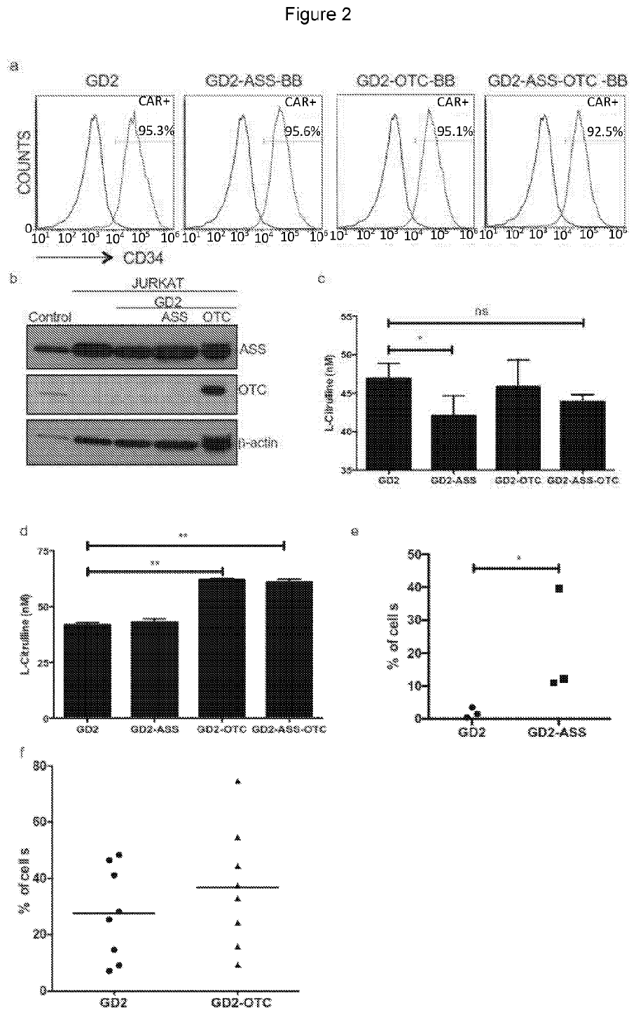

The inventors investigated the role of arginine pathway enzymes in an immortalised line of human T lymphocyte cells (Jurkat cells). The results of this study are set out in FIG. 2.

The Jurkat cells were transduced with fusion target-binding protein constructs comprising the arginine pathway enzymes, or control CAR-T constructs. For experimental purposes both the cells of the invention and control cells were transduced to express proteins comprising a GD2-binding moiety. The purity of the protein-enzyme constructs produced was assessed by measuring expression of tCD34 using flow cytometry. The results show that the protein-enzyme constructs can be produced to a high degree of purity in the Jurkat cells, shown in FIG. 2a.

There was an increase in the expression of ASS-land OTC in the transduced cells (note Jurkat has a higher background ASS-1 expression compared to primary PBMCs), shown in FIG. 2b.

The inventors investigated the ability of the domains that promote the synthesis of arginine in the transduced cells to perform their function.

The catabolism of citrulline to arginosuccinate by ASS-1 was assessed and compared to control constructs, (without an ASS-1 domain, GD2-CAR), a fusion target-binding protein containing an OTC domain (GD2-OTC) and fusion target-binding protein containing both ASS-1 and OTC domains (GD2-ASS-OTC). The lysates of the GD2-ASS-transduced Jurkat cell were tested in a colorimetric assay for activity of ASS-1 enzyme to directly catabolise citrulline into argininosuccinate. The fusion target-binding protein containing an ASS-1 domain that promotes the synthesis of arginine demonstrated GD2-ASS-1 fusion target-binding proteins have a significantly higher ASS-1 activity than the cells containing the control fusion target-binding protein constructs. Shown in FIG. 2c.

The catabolism of ornithine into citrulline by OTC was assessed and compared to control constructs, without an OTC domain (GD2 only), a fusion target-binding protein containing an ASS-1 domain (GD2-ASS) and fusion target-binding protein containing both ASS-1 and OTC domains (GD2-ASS-OTC). The lysates of the GD2-OTC-transduced Jurkat cells were tested in a colorimetric assay for the activity of OTC enzyme to directly catabolise ornithine into citrulline. The fusion target-binding protein containing an OTC domain (GD2-OTC) and the fusion target-binding protein containing an ASS-1 and an OTC domain (GD2-ASS-OTC) constructs had a significantly higher OTC activity that the cells containing the control CAR constructs, shown in FIG. 2d.

The inventors investigated the persistence of fusion target-binding protein T cells transduced with constructs comprising domains that promote the synthesis of arginine in a tumour microenvironment.

NOG-SCID mice were engrafted with 5×10⁶ fusion target-binding protein T cells. Recombinant-PEG-arginase was administered twice weekly to create a reproducible, low arginine microenvironment (confirmed on arginine ELISA). Mice were sacrificed and the percentage of fusion target-binding protein T cells in the blood were measured by flow cytometry. Fusion target-binding protein T cells comprising an ASS-1 domain (GD2-ASS) showed a significantly enhanced persistence compared to fusion target-binding proteins without the ASS-1 domain (GD2-CAR T cells), shown in FIG. 2e.

Fusion target-binding protein T cells comprising an OTC domain (GD2-OTC) showed a significantly enhanced persistence compared to T cells without the fusion target-binding protein comprising the OTC domain (GD2-CAR T cells), shown in FIG. 2f.

3 Arginine Pathway Enzymes can be Transduced into PBMCs from Human Donors.

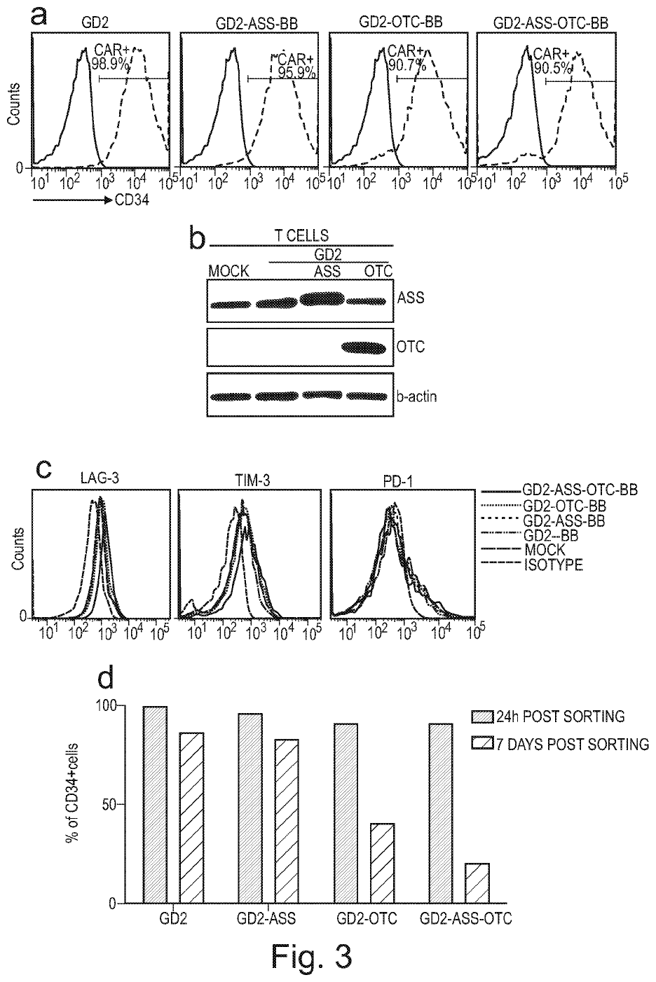

The inventors investigated the role of arginine pathway enzymes in PBMCs from human donors cells). The results of this study are set out in FIG. 3.

The PBMCs were transduced with fusion target-binding protein constructs comprising the arginine pathway enzymes. The purity of the CAR-enzyme constructs produced was assessed by measuring expression of tCD34 using flow cytometry. The results show that the fusion target-binding protein-enzyme constructs can be produced to a high degree of purity in the PBMCs, shown in FIG. 3a.

There was an increase in expression of ASS-1 and OTC is increased in transduced cells, shown in FIG. 3b.

The inventors found that there were no differences in expression of the co-inhibitory receptors LAG-3, TIM-3, and PD-1 in CAR-T cells also containing the constructs comprising a domain that promote the synthesis of arginine.

The persistence of PBMCs transduced with the constructs comprising a domain that promotes the synthesis of arginine was measured during a 7 day expansion, as detected by flow cytometry of tCD34. GD2-ASS-1 construct demonstrated similar persistence to the GD2 alone construct. Over time the GD2-OTC and GD2-ASS-OTC constructs were not maintained, as shown in FIG. 3d. These experiments were performed in normal arginine conditions with no antigen present. The inventors hypothesise that a survival advantage may be seen in low arginine conditions and in the presence of an antigen. Any loss of cells occurring in vivo may be overcome by repeated administration of cells of the invention to a patient, with the interval of administration reflecting the time for which cells survive in the body.

### 4 ASS-1 and OTC Confers a Significant Metabolic and Proliferative Advantage in Low Arginine Tumour Conditions

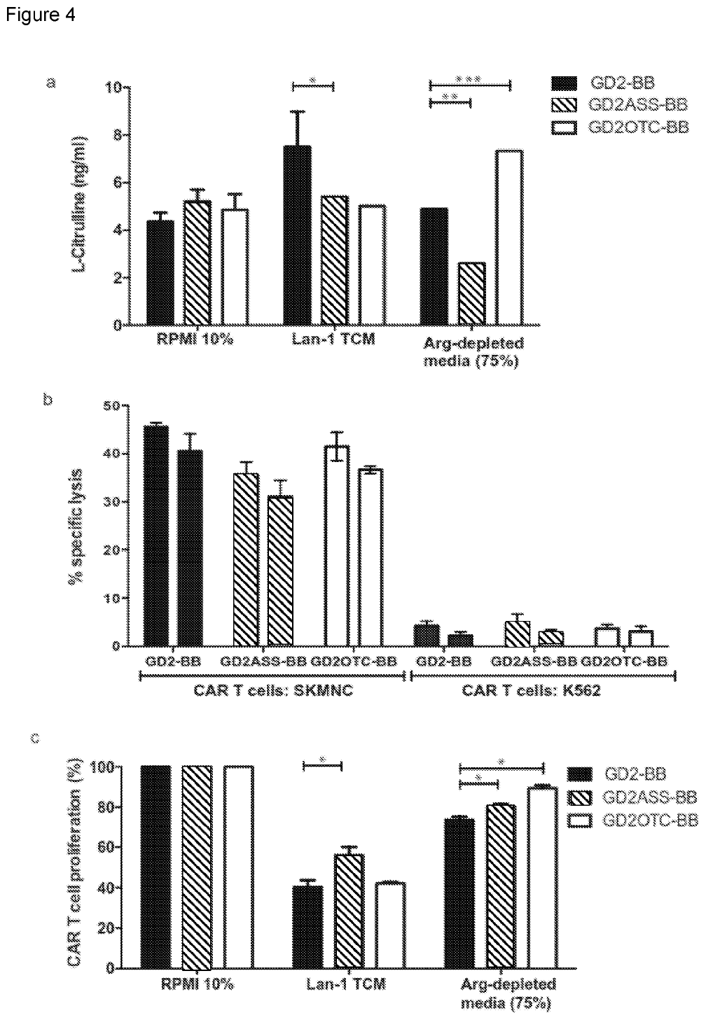

The ability of fusion target-binding protein T cells comprising a domain that promotes synthesis of arginine, (GD2-ASS, and GD2-OTC) to enhance citrulline metabolism when cultured in normal arginine (RPMI+10% FCS), LAN-1 neuroblastoma conditioned supernatant, and 75% arginine depleted media conditions was detected by ELISA of culture supernatants. Under tumour-derived low arginine conditions the GD2-ASS-1 fusion target-binding proteins and GD2-OTC fusion target-binding proteins significantly upregulated citrulline metabolism consistent with enzyme expression and activation compared to the control (without a domain that promotes the synthesis of arginine, GD2 only), shown in FIG. 4a.

The specific cell lysis of fusion target-binding proteins comprising an ASS-1 domain (“GD2-ASS-BB”), and an OTC domain (“GD2-OTC-BB”) fusion target-binding protein T cells on neuroblastoma cell line and myeloenous leukaemia cell line was assessed against the control (GD2 only: “GD2-BB”). The fusion target-binding protein T cells were cultured in the presence of chromium labelled GD2+ LAN-1 neuroblastoma cells at different effector to target ratios for 4 hours. All CAR-T cell constructs specifically kill neuroblastoma cells effectively (35-45%) consistent with the inventors' previous data. No detriment to cytotoxicity is seen with the addition of ASS-1 or OTC. Specificity of this cytocidal activity was demonstrated by the fact that GD2-CAR T cells (whether control or of the invention) had minimal killing of the myelongenos leukaemia cell line (GD2-K562) (<5% specific lysis). Shown in FIG. 4b.

CAR T cells comprising a domain that promotes synthesis of arginine (ASS-1 or OTC) showed a significant rescue of proliferation in low arginine conditions. CAR-T cells were cultured in normal arginine (RPMI+10% FCS) neuroblastoma-derived low arginine supernatants, or 75% arginine deplete media. T cell proliferation was measured by tritiated-thymidine incorporation after 96 hours. CAR-T cells show a reduction in T cell proliferation in low arginine conditions, consistent with our previous findings. The cells transfected with a construct comprising an ASS-1 domain (GD2-ASS) and an OTC domain (GD2-OTC) showed a significant rescue of proliferation in these conditions compared to the control (GD2-CAR), as shown in FIG. 4c.

5 Modified CAR-T Cells have Enhanced Anti-Tumour Activity In Vivo and can be Applied to Non-GD2 CAR-T Cells

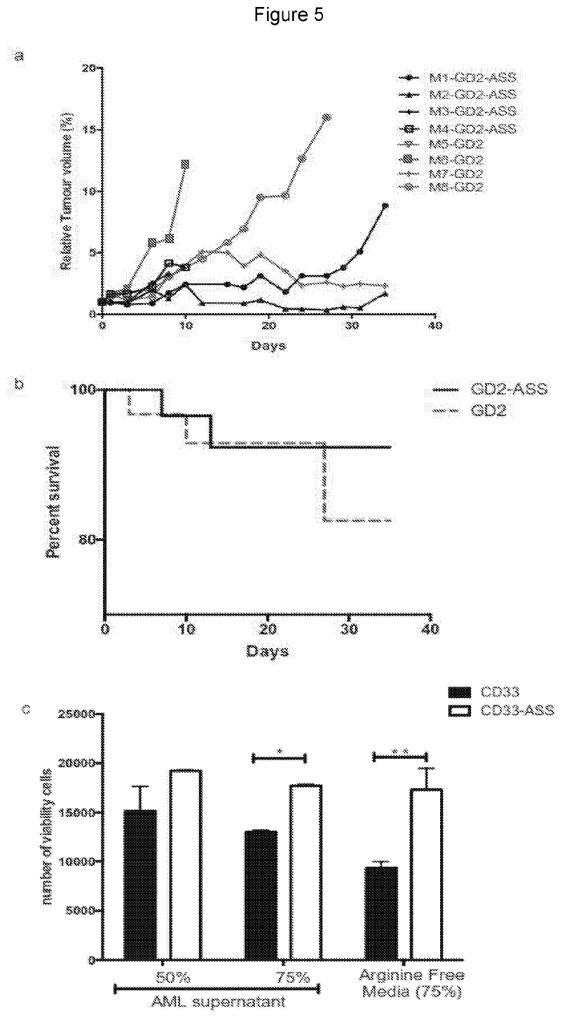

NOG-SCID mice engrafted with GD2+ tumour cells. CAR-T cells comprising an ASS-1 domain (GD2-ASS) and without (GD2-CAR) were administered via tail vein injection. The relative tumour growth was measured over time. Administration of GD2-ASS-1 CAR T cells led to a reduction in tumour growth, compared to GD2-CAR T cells, as shown in FIG. 5a.

Administration of GD2-ASS-1 CAR T cells also led to improved murine survival, as shown in FIG. 5b. The results shown in this figure illustrate not only the cytocidal (and hence therapeutic) activity of the cells of the invention, but also their improved persistence in vivo, since this therapeutic activity is observed over a longer period of time than for the control cells.

The viability of CD33 and CD33-ASS-1 CAR T cells were assessed. CD33-ASS-1 CARs were cultured in AML cell line condition media (low in arginine) or 50-75% arginine depleted media. CD33-ASS-1 CARs showed significantly enhanced viability in low arginine conditions compared to CD33 CARs. Results illustrating this are shown in FIG. 5c.

### 6 Increased Persistence of Cells of the Invention in Arginine-Depleted Conditions In Vivo.

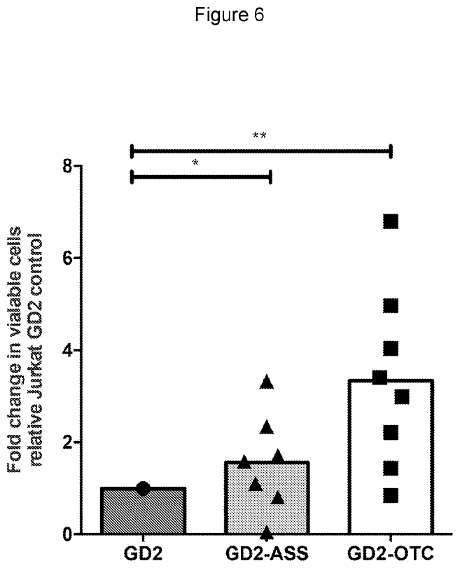

The improved persistence of the cells of the invention was demonstrated in NOG-SCID mice engrafted with 5×10⁶ anti-GD2 CAR-T Jurkat cells (control cells), or Jurkat cells expressing proteins of the invention comprising a GD2 target moiety and an ASS-1 domain (GD2-ASS), or cells expressing proteins of the invention comprising a GD2 target moiety and an OTC domain (GD2-OTC), administered intravenously. Recombinant-PEG-arginase was administered twice weekly to mice in order to create a reproducible, low arginine microenvironment (to replicate a tumour microenvironment). The low arginine conditions were confirmed by ELISA (data not shown). After 17 days mice were sacrificed and the percentages of CAR-T cells in the blood (whether control or of the invention) were measured by flow cytometry. The GD2-ASS-1 and GD2-OTC CAR-T cells showed significantly enhanced persistence as compared to the control cells comprising the unmodified GD2 CAR-T construct. These results are shown in FIG. 6.

7 Arginine Pathway Enzymes can be Transduced into PBMCs from Human Donors Comprising Various Target Binding Moieties.

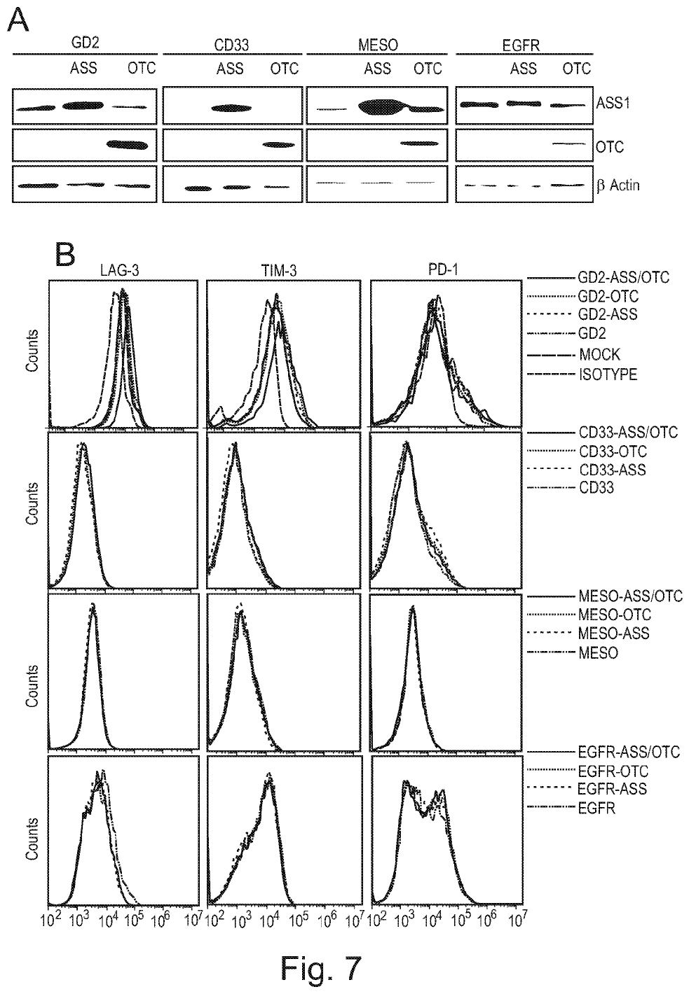

PBMCs (specifically T cells) from human donors were transduced with proteins of the invention comprising an ASS-1 domain and/or an OTC domain in combination with target binding moieties selected from the list consisting of: GD2, CD33, Mesothelin, or EGFRvIII. Western blots show that expression of ASS-1 and OTC is increased in cells transduced with proteins of the invention compared to a control cell (left hand column of each Western blot). This is show in FIG. 7a.

Expression of LAG-3, TIM-3, and PD-1 (co-inhibitory receptors of potential importance in the treatment of cancer) was also assessed by flow cytometry in the transduced CAR-T cells expressing the proteins of the invention comprising: an ASS-1 domain; an OTC domain; or an ASS-1 domain and OTC domain. Exemplars for anti-GD2, anti-CD33, anti-MESO, and anti-EGFRvIII CAR-T cells shown in FIG. 7 panel B.

### 8 Cytocidal Activity of Cells Expressing a CD33 Targeting Domain

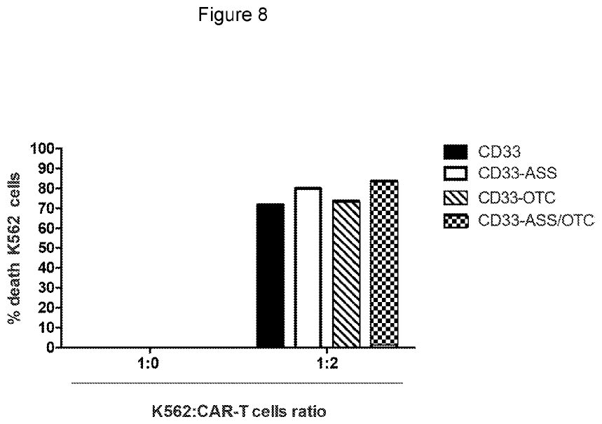

CAR-T cells of the invention were produced by expression of proteins of the invention comprising a CD33 targeting domain in combination with either an ASS-1 domain, an OTC domain or an ASS-1 and OTC domain. The CAR T cells were cultured in the presence of K562 leukaemia cells at different effector to target ratios for 4 hours. All CAR-T cell constructs specifically kill leukaemia cells effectively (70-90%). Transduction of CD33 CAR T-cells with the proteins of the invention do not detrimentally impact the cytotoxicity of the CAR T-cells, shown in FIG. 8. The ability of cells comprising proteins of the invention to maintain cytocidal activity, while also demonstrating increased persistence and proliferation, as shown in the rest of the Examples, illustrates their therapeutic utility.

### 9 Enzyme Modifications Confer a Significant Metabolic and Proliferative Advantage in Low Arginine Tumour Conditions.

Cells were transduced to express one of the following proteins of the invention:
A protein comprising a GD2-binding domain and an ASS-1 domain
A protein comprising a GD2-binding domain and an OTC domain
A protein comprising a GD2-binding domain and both an ASS-1 and an OTC domain
A protein comprising a CD33-binding domain and an ASS-1 domain
A protein comprising a CD33-binding domain and an OTC domain
A protein comprising a CD33-binding domain and both an ASS-1 and an OTC domain
A protein comprising a mesothelin-binding domain and an ASS-1 domain
A protein comprising a mesothelin-binding domain and an OTC domain
A protein comprising a mesothelin-binding domain and both an ASS-1 and an OTC domain
A protein comprising an EGFRvIII-binding domain and an ASS-1 domain
A protein comprising an EGFRvIII-binding domain and an OTC domain
A protein comprising an EGFRvIII-binding domain and both an ASS-1 and an OTC domain

They were cultured in low arginine conditions (75% arginine depleted complete media). Unmodified CAR-T cells sharing the same binding domains (i.e. anti-GD2, anti-CD33, anti-mesothelin, or anti EGFRvIII), but lacking the enzyme domains, were used as controls. Proliferation of all cells was measured by flow cytometry after 96 hours.

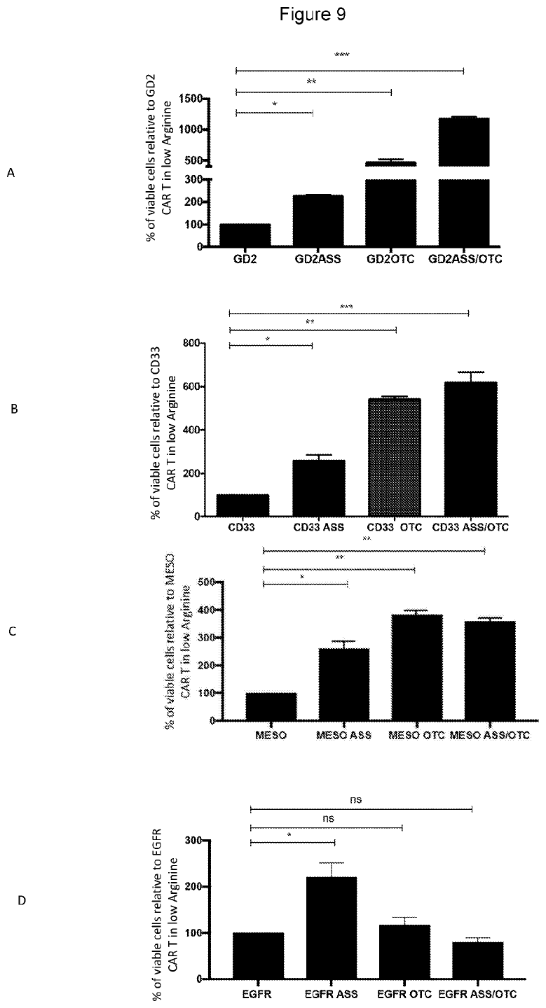

In the case of anti-GD2 cells, the addition of an ASS-1 domain, an OTC domain or ASS-1 and OTC domain significantly enhanced CAR-T cell proliferation compared to the unmodified control CAR-T cells. In fact, the GD2-OTC CAR T-cells demonstrate a 5-fold increase in proliferation compared to the GD2 only control cell. Additionally, the GD2-ASS/OTC CAR T cells demonstrate a 10-fold increase in proliferation compared to the GD2-only CAR T cell control. Shown in FIG. 9 panel A.

In the case of anti-CD33 CAR-T cells, the addition of an ASS-1 domain, an OTC domain or ASS-1 and OTC domain significantly enhanced CAR-T cell proliferation compared to the unmodified control CAR-T cells. The CD33-OTC CAR T-cells demonstrate approximately a 5-fold increase in proliferation compared to the CD33 only control cell. Additionally, the CD33-ASS/OTC CAR T cells demonstrate a 6-fold increase in proliferation compared to the GD2-only CAR T cell control. Shown in FIG. 9 panel B.

For anti-mesothelin CAR-T cells, the addition of an ASS-1 domain, an OTC domain or ASS-1 and OTC domain significantly enhanced CAR-T cell proliferation compared to the unmodified control CAR-T cells. The mesothelin-OTC CAR T-cells demonstrate approximately a 4-fold increase in proliferation compared to the CD33 only control cell. Additionally, the mesothelin—ASS/OTC CAR T cells demonstrate approximately a 3.5-fold increase in proliferation compared to the mesothelin-only CAR T cell control. Shown in FIG. 9 panel C.

In anti-EGFRvIII CAR-T cells, the addition of an ASS-1 domain, significantly enhanced proliferation by approximately 2.5-fold compared to the unmodified control CAR-T cells Shown in FIG. 9 panel D.

### 10 Enzyme Modifications Confer a Significant Metabolic and Proliferative Advantage in Tumour Conditioned Medium (TCM).

Cells expressing proteins of the invention comprising a GD2-binding moiety in combination with either: an ASS-1 domain, an OTC domain, or an ASS-1 and OTC domain, were cultured in neuroblastoma tumour conditioned media. Such media have low arginine conditions, due to the action of the tumour cells. Anti-GD2 CAR-T cells without enzyme domains were used as control cells.

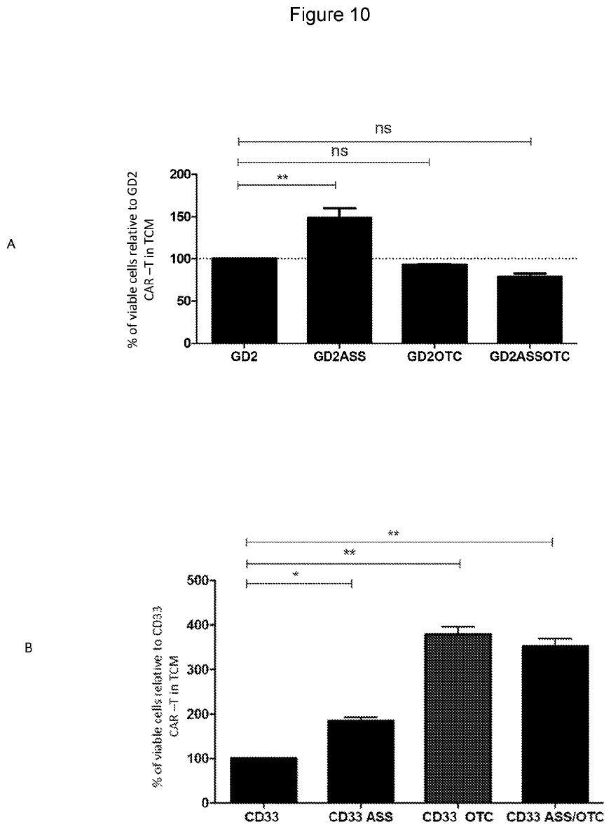

Proliferation of the cultured cells was measured by flow cytometry after 96 hours. The addition of a protein of the invention comprising an ASS-1 domain significantly enhanced CAR-T cell proliferation compared to the unmodified control CAR-T cells, as shown in FIG. 10 panel A.

Cells expressing proteins of the invention comprising a CD33-binding moiety in combination with either: an ASS-1 domain, an OTC domain, or an ASS-1 and OTC domain, were cultured in leukaemia tumour conditioned media (which also contains low levels of arginine). In this case anti-CD33 CAR-T cells without enzyme domains were used as control cells, and cell proliferation was (again) measured by flow cytometry after 96 hours.

The addition of a protein of the invention comprising an ASS-1 domain, OTC domain or an ASS-1 and OTC domain significantly enhanced CAR-T cell proliferation compared to the unmodified control CAR-T cells. The CD33 CAR T cells comprising an OTC domain demonstrate an approximately a 4-fold increase in proliferation. The CD33 CAR T cells comprising an ASS-1 and an OTC domain demonstrate approximately a 3.5-fold increase in proliferation compared to the unmodified control CAR T cell. These results are shown in FIG. 10 panel B.

### 11 Enzyme Modification Significantly Enhance Anti-Tumour Activity In Vivo

HL-60 acute myeloid leukaemia (AML) cells were engrafted into NOG-SCID mice. Leukaemia bearing mice were treated with cells expressing proteins of the invention comprising a CD33-binding moiety and either: an ASS-1 domain, an OTC domain, or an ASS-1 domain and an OTC domain. Anti-CD33 CAR-T cells lacking an enzyme domain (unmodified CAR-T cells) were used as controls. The cells of the invention or control cells were administered intravenously at a dose of 5×10⁶ cells.

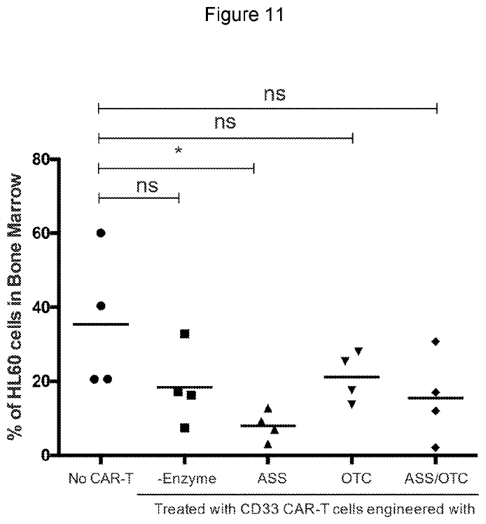

As shown in FIG. 11, CAR-T cells expressing a protein of the invention comprising an ASS-1 domain (Anti-CD33-ASS-1 CAR-T cells) significantly enhanced AML clearance from the bone marrow, as compared to control CAR-T cells.

### 12 Enzyme Modification of GD2 CART T Cells Significantly Enhance Anti-Tumour Activity In Vivo

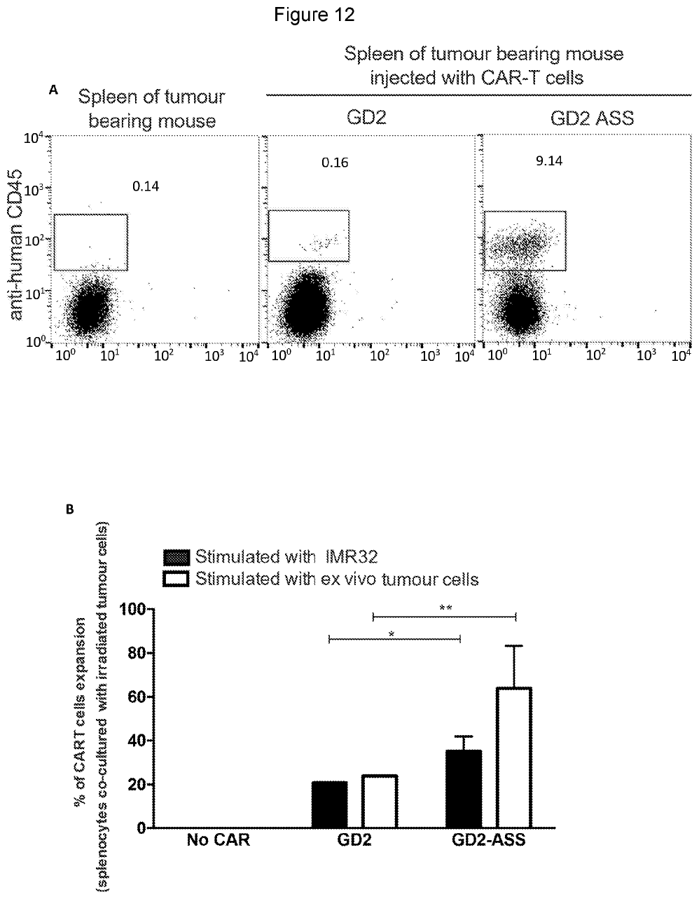

Neuroblastoma xenograft mice were treated with cells expressing a protein of the invention comprising a GD2-binding moiety and an ASS-1 domain. Control animals received either GD2 CAR-T cells (without an ASS-1 domain), or no CAR-T treatment. Spleens of all animals were harvested and extracted leukocytes characterised by flow cytometry. The results are shown in Panel A of FIG. 12, and illustrate that cell numbers were markedly increased for the cells of the invention, as compared to control cells. This indicates that the cells of the invention (expressing a protein of the invention comprising an ASS-1 domain) have improved persistence in the spleens of the treated mice, as compared to controls.

Extracted leukocytes were also co-cultured with neuroblastoma target cells (IMR32 cell line or tumour cells) ex vivo to investigate the ability of cells of the invention that have persisted in the recipient to undergo expansion in response to antigen stimulation. Results are shown in Panel B of FIG. 12. It can be seen that the numbers of the cells of the invention are significantly higher than those of the controls. This indicates that the persistent cells of the invention retain an ability to proliferate in response to antigen stimulation that is greater than that of the controls.

### 13 Enzyme Modification of CD33 CART T Cells Significantly Enhance Anti-Tumour Activity In Vivo

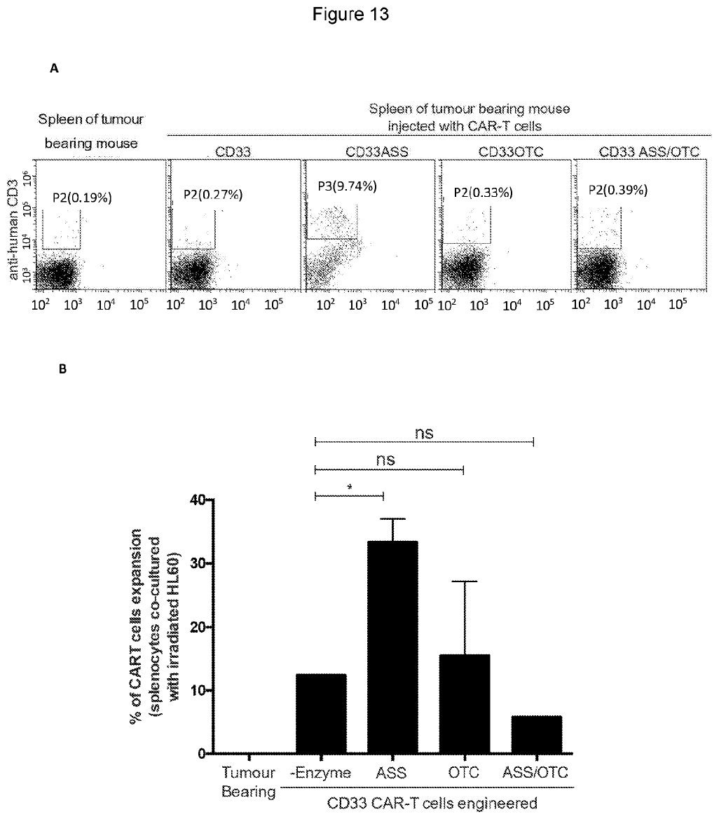

AML xenograft mice were treated with cells expressing a protein of the invention comprising a CD33-binding moiety and one of: an ASS-1 domain, an OTC domain; or an ASS-1 domain and an OTC domain. Control groups received either CD33 CAR-T cells (without an enzyme domain—shown as “-enzyme”), or no CAR-T treatments. Spleens of all animals were harvested and extracted leukocytes were characterised by flow cytometry. The results, shown in Panel A of FIG. 13, illustrate that cell numbers were markedly increased for the cells of the invention, as compared to control cells. This indicates that the cells of the invention also have improved persistence in the spleens of the treated mice (as compared to controls) in the context of AML.

The extracted leukocytes were also co-cultured with AML target cells ex vivo to investigate the ability of cells of the invention that have persisted in the recipient to undergo expansion in response to antigen stimulation. Results are shown in Panel B of FIG. 13. The numbers of the cells of the invention are significantly higher than those of the controls, particularly in the case of the cells expressing a protein of the invention with and ASS-1 domain. These results indicate that the cells of the invention that have persisted within the recipient retain an ability to proliferate in response to antigen stimulation that is greater than that of the control cells.

### Protocols for the Production of Cells of the Invention

Cells of the invention have been successfully produced by retroviral and by lentiviral transduction approaches. Details of an exemplary protocol for the retroviral production of cells of the invention are set out below.

### Retroviral Transduction of Human T Cells

The following provides a protocol for the production of cells of the invention by transfection with nucleic acids of the invention.

### Day −2: Thaw Phoenix Ampho Cells

Late afternoon get Phoenix Ampho cells (retroviral packaging cell line for transduction of human cells) out of −80 and place in culture. Phoenix Ampho cells are grown in DMEM with 10% FCS, 1% L-glut (no antibiotics). Phoenix Ampho cells should never reach confluency. Typically put 2-3×10⁶ Phoenix Ampho cells in each T150 flask in 30 ml of media. On day 0 you should have around 30-40×10⁶ Phoenix Ampho cells.

### Day 1: Set Up Phoenix Ampho Cells

Trypsinise Phoenix Ampho cells using TryLE and set up Phoenix Ampho cells at 8×10⁶ cells/flask in 30 ml DMEM with 10% FCS and 1% L-glutamine (no antibiotics) (volume for T150 flask, scale as appropriate). Incubate cells overnight (37° C./5%002).

### Day 2: Transfection of Phoenix Ampho Cells

Phoenix Ampho cells should be 50-80% confluent on the day of transfection. The cells should then be transfected by the following method (for a T150 flask, scale as appropriate if using different flasks).
1. For each T150 flask of phoenix cells, place 12 μg of plasmid DNA (i.e. CAR plasmid)+12 μg pCI ampho plasmid into a 15 ml falcon and make up to 1800 μl with OptiMEM (Gibco) mixing gently with a pipette. To another 15 ml falcon add 1680 μl OptiMEM and add 120 μl Fugene 6 transfection reagent (available from stores) ensuring Fugene goes directly into OptiMEM rather than sticking to sides of tube; mix gently with a pipette. Then add the 1800 μl of OptiMEM/fugene mix to the tubes containing the plasmid DNA and mix gently with a pipette. Incubate at room temp for 45 mins. This allows fugene to form complexes with the DNA that have a neutral charge allowing DNA to be transported across the negatively charged Phoenix Ampho cell membrane.
2. Very gently replace the media on the Phoenix Ampno cells to 9 mls fresh DMEM with 10% FCS and glutamine then immediately overlay the DNA/fugene complexes or OptiMEM (for mock controls) onto the cells. Gently mix by north-south and east-west movements of the plate.
3. Incubate cells for 24 hours (37° C./5% CO₂).

### Day 2: Activation of T Cells

T cells will not expand in the first 48 hours after activation, so typically activate as many T cells as you need (or more in case of cell death) for your transduction.

### Method Using Anti-CD3/CD28 Antibodies:

1. Lymphoprep a fresh leukocyte cone.
2. Count cells and culture at 1×10⁶/ml in T cell media (1% human serum, 10% FCS, P/S, L-glut RPMI). Typically 200 mls per T150 flask.
3. Add IL-2 at 300 U/ml, add OKT3 (anti-CD3) at 30 ng/ml, add anti-CD28 mAB at 30 ng/ml (#MAB342-SP, R&D).
4. Incubate at 37° C./5% CO2 for 48 hours.

### Method Using Anti-CD3/CD28 Dynabeads:

1. Lymphoprep a fresh cone. Count cells and assume that 50% of PBMCs are CD3+ T cells.
2. Resuspend cells in a 15 ml falcon at 10×10⁶ CD3+ T cells per ml of 5% human serum, PBS.
3. Add two Dynabeads® Human T-Activator CD3/CD28 per CD3+ cell. Washing dynabeads: vortex vial of beads for 30s. Remove required volume of beads and place in a 15 ml falcon. Add 1 ml of sterile PBS and mix well with a pipette. Place falcon on the dynabead magent—dynabeads will stick to the edge of the falcon. Carefully remove the supernatant without disturbing the beads. Take falcon off the magnet and repeat wash step. Add dynabeads to your T cells in a small volume of PBS.
4. Incubate T cells on a tumbler at room temperature for at least an hour. T cells will bind dynabeads during this step, allowing selection of CD3+ T cells and activation at the same time.
5. Place cells on the dynabead magnet to remove non-bound cells. Count cells and culture at 1×10⁶/ml in T cell media (1% human serum, 10% FCS, P/S, L-glut RPMI) with IL-2 300U/ml
6. Incubate at 37° C./5% CO2 for 48 hours.

### Day 3: Change Phoenix Ampho Media

Phoenix ampho cells will now be producing retrovirus containing your plasmid DNA, so take this into account when handling cells/supernatants. Place an autoclave bag inside your TC hood and place any plastics contaminated with retrovirus (cells/sups) within it. When you are finished, seal the autoclave bag and place into an autoclave tin. Put any liquid waste in a waste pot and seal. Take retrovirus contaminated waste to wash-up ASAP.

Gently replace medium on Phoenix Ampho cells with fresh 21 mls/flask (volume for T150 flask, scale as appropriate) of DMEM+10% FCS+2 mM L-glutamine (no antibiotics). Incubate the cells for a further 24 hours.

### Day 4: Transduction of Human T-Cells

1. Add 2 mls of retronectin (30 μg/ml) (#T100B—Takara RetroNectin® Recombinant Human Fibronectin Fragment) to each of the required number of wells of a 6-well plate (non tissue culture-treated) and incubate at room temperature for 3 hours (can also be set up the day before and coated overnight in the fridge). Remember to include wells for mock-transfected controls in the experiment. Culture plates are coated in retronectin to co-localise T cells and virus particles to allow efficient transduction of cells
2. Remove retronectin (it can be re-used until it has run out) and block wells with 2.5 ml of sterile PBS/2% BSA/well for 30 mins. Remove the blocking solution and wash wells twice with 2.5 mls of sterile PBS (keep last PBS wash on well until ready to add virus).
3. Pre-warm some T cell media.
4. Pre-warm centrifuge for spinfection by spinning with empty buckets at 3160 rpm/2000g for 60 mins @ 32° C. This can be interrupted when ready to do spinfection.
5. Harvest retrovirus-containing culture supernatant from Phoenix Ampho cells and spin down (1500 rpm for 5 mins). Transfer retrovirus sup to fresh tubes. Some people filter their retrovirus using a 0.45 μm filter, to remove comtaminating Phoenix ampho cells, but this could decrease retroviral titre. If necessary, the virus can be snap frozen on dry ice/ethanol slurry and stored at −80° C., but titre is halved with every freeze thawing.
6. Spinfection: Add 2 ml/well of virus supernatant (or mock supernatant) to retronectin-coated wells and spin at 3160 rpm/2000 g for 2 hours @ 32° C.
7. 45 mins before this spin finishes, prepare the T cells due to be transduced. Harvest T cells and count. Resuspend T cells at 1×10⁶ in T cell media+IL2 (100U/ml) and incubate (37° C./5% CO₂) for 15-20 mins to allow cells to recover from centrifugation.
8. When virus has finished spinning, remove supernatants and wash wells once with PBS (2.5 ml/well).
9. Remove PBS from virus/retronectin coated plate and add T cells due to be transduced (2 ml/well). Ensure cells evenly distributed over plate by rocking north:south and east:west. Spin plates at 1300 rpm for 5 mins.
10. Place plates in incubator (37° C./5% CO₂).

### Day 5: Feed Transduced T Cells

Add another 6 ml of T cell media+IL2 (100 IU/ml) to each well of T cells and return to incubator (37° C./5%002).

### Determining CAR Transduction Efficiency

The efficiency of methods for transducing cells to produce cells of the invention may be determined using the following procedure.

CAR T cell transduction efficiency is determined 4 days post-spinfection. Take samples from mock and CAR T cell wells and stain as follows:
1. Wash ×1 with FACs buffer (10% FCS, PBS)
2. Stain with CD34-APC (1 μl/sample), CD4-FITC (2 μl/sample) and CD8-PE (1 μl/sample) in 50 μl of FACs buffer
3. Incubate for 20 mins on ice
4. Wash ×1 with FACS buffer
5. Resuspend cells in 200 μl of FACs buffer and analyse by flow cytometry (LSRII).

### Sorting Cells of the Invention (Such as CAR T Cells) by CD34 Magnetic-Activated Cell Sorting

CAR-transduced cells (such as T cells) are sorted as follows:
1. Spin down T-cells at 1500 rpm, 5 mins and pour off supernatant.
2. Resuspend cells in 10 ml cold MACS buffer and spin 1500 rpm, 5 mins and pour off supernatant.
3. Resuspend cells in 300 μl cold MACS buffer, add 100 μl FcR blocking agent and 100 μl CD34 microbeads (Miltenyi Biotech 130-046-702). These quantities are suitable for up to 10⁸ cells—if more than that, scale up accordingly.
4. Mix well and incubate for 30 mins in the fridge (2-8° C.).
5. Wash in 50 ml cold MACS buffer and spin 1500 rpm, 5 mins and pour off supernatant.
6. Resuspend cells in 500 μl cold MACS buffer and load cell suspension onto an MS column that has been pre-rinsed with 500 μl cold MACS buffer.
7. Allow cells to drip through by gravity flow and wash columns 3 times with 500 μl cold MACS buffer.
8. Remove columns from the magnet and flush through with 1 ml cold MACS buffer, collecting the cells in a sterile tube.
9. Centrifuge sorted CAR T cells and resuspend in normal T cell media (1% human serum, 10% FBS, P/S, L-glut, 100U/ml IL-2, RPMI 1640) at a concentration of 1×10⁶ CAR T cells per ml.
10. Check purity of CAR T cells by CD34 surface antibody staining the following day.

### Assay to Determine Arginosuccinate Synthase (ASS-1) or Arginosuccinate Synthetase (ArgG) Enzyme Activity—L-Citrulline Depletion

1. Pellet 5×10⁶ cells per sample (if you have enough cells pellet at addition 5×10⁶ cells for a no substrate control).
2. Resuspend in 20 μl of 0.1% Triton-X+protease inhibitors (0.1% Triton-X is stored in a 50 ml tube at 4° C. in molecular lab), and incubate on ice for 20 minutes with occasional vortexing.
3. Centrifuge samples at 13,000 rpm for 20 minutes at 4° C. to pellet cell debris.
4. Take 20 μl of cell lysate in to fresh eppendorfs and keep on ice.
5. Take a fresh eppendorf for each sample and add 10 μl of L-Citrulline (4 mM, pH 7.5), 10 μl of L-Aspartic Acid (4 mM, pH 7.5), 10 μl of MgCl₂ (6 mM), 10 μl of ATP (4 mM, pH 7.5), 40 μl of Tris-HCl (20 mM) and 20 μl of cell lysate.
6. For a no enzyme control, add 20 μl of 0.1% Triton-X+protease inhibitors instead of cell lysate.
7. If performing no substrate controls do not add the ASS-1 or ArgG substrates i.e. L-Citrulline and L-Aspartic acid. Instead make up to a final volume of 100 μl with Tris-HCl (20 mM).
8. Incubate samples at 37° C. for 1.5 hrs. ASS-1 or ArgG enzyme reaction will occur.
9. Make L-Citrulline standards by making a 1 mM L-Citrulline solution by performing a 1:4 dilution of the 4 mM L-Citrulline solution. To eppendorfs add 10, 20, 30, 50, 80 and 100 μl of L-Citrulline (1 mM), and make each standard up to 100 μl with sterile distilled water. This makes L-Citrulline concentrations of 0, 10, 20, 30, 50, 80 and 100 nM. Include a blank control.
10. To each standard add 10 μl of L-Aspartic Acid (4 mM, pH 7.5), 10 μl of MgCl2 (6 mM), 10 μl of ATP (4 mM, pH 7.5) and 20 μl of 0.1% Triton-X+protease inhibitors. ASS-1 or ArgG enzyme reagents are added to standards to more accurately determine L-Citrulline concentrations within ASS-1 or ArgG enzyme activity samples.
11. After 1.5 hrs at 37° C., add 80 μl of stop solution (3:1 mix of phosphoric acid and sulfuric acid respectively) and 20 μl of 3% 2, 3 butanedione monoxime (made fresh on the day with sterile distilled water and in a fume cupboard because it smells awful) to each sample and standard.
12. Mix tubes by vortexing and pulse centrifuge. Incubate all samples and standards at 95° C. for 30 minutes. A yellow/orange colour will appear as 2, 3 butanedione monoxime reacts with L-Citrulline in acidic conditions at 95° C.
13. Centrifuge tubes at 13000 rpm for 1 minute to pellet any debris. In duplicate, add 50 μl of supernatant to wells of a 96-well flat bottom plate.
14. Read absorbance at 490 nm using the microplate absorbance reader.

### Assay to Determine Ornithine Transcarbamylase (OTC) or Ornithine Carbamoyltransferase (ArgF) Enzyme Activity Assay—L-Citrulline Production

1. Pellet 5×10⁶ cells per sample (if you have enough cells pellet at addition 5×10⁶ cells for a no substrate control).
2. Resuspend in 20 μl of 0.1% Triton-X+protease inhibitors (0.1% Triton-X is stored in a 50 ml tube at 4° C. in molecular lab), and incubate on ice for 20 minutes with occasional vortexing.
3. Centrifuge samples at 13,000 rpm for 20 minutes at 4° C. to pellet cell debris.
4. Take 20 μl of cell lysate in to fresh eppendorfs and keep on ice.
5. Take a fresh eppendorf for each sample and add 25 μL of L-Ornithine (50 mM, pH 8.0), 25 μl of triethanolamine (pipette straight from the bottle, solution is very viscous so pipette slowly), 25 μL of freshly prepared Carbamyl Phosphate (150 mM, pH 8.0), and 10 ul of SDW.
6. Then add 20 ul of cell lysate to each tube. For a no enzyme control, add 20 μl of 0.1% Triton-X+protease inhibitors instead of cell lysate.
7. If performing no substrate controls do not add the OTC or ArgF substrates i.e. L-Ornithine and Carbamyl Phosphate. Instead make up to a final volume of 100 μl with SDW.
8. Incubate samples at 37° C. for 1.5 hrs. OTC or ArgF enzyme reaction will occur.
9. Make L-Citrulline standards by making a 1 mM L-Citrulline solution by performing a 1:4 dilution of a 4 mM L-Citrulline solution (stocks in −20 used for ASS-1 enzyme activity assay). To eppendorfs add 10, 20, 30, 50, 80 and 100 μl of L-Citrulline (1 mM), and make each standard up to 100 μl with sterile distilled water. This makes L-Citrulline concentrations of 0, 10, 20, 30, 50, 80 and 100 nM. Include a blank control.
10. To each standard add 25 μL of L-Ornithine (50 mM), 25 μl of triethanolamine, 25 μL of freshly prepared Carbamyl Phosphate (150 mM) and 20 μl of 0.1% Triton-X+protease inhibitors. OTC enzyme reagents are added to standards to more accurately determine L-Citrulline concentrations within OTC or ArgF enzyme activity samples.
11. After 1.5 hrs at 37° C., add 80 μl of stop solution (3:1 mix of phosphoric acid and sulfuric acid respectively) and 20 μl of 3% 2, 3 butanedione monoxime (made fresh on the day with sterile distilled water and in a fume cupboard because it smells awful) to each sample and standard.
12. Mix tubes by vortexing and pulse centrifuge. Incubate all samples and standards at 95° C. for 30 minutes. A yellow/orange colour will appear as 2, 3 butanedione monoxime reacts with L-Citrulline in acidic conditions at 95° C.
13. Centrifuge tubes at 13000 rpm for 1 minute to pellet any debris. In duplicate, add 50 μl of supernatant to wells of a 96-well flat bottom plate.
14. Read absorbance at 490 nm using the microplate absorbance reader.

### Assay to Determine Argininosuccinate Lysase (ASL) or (ArgH) Enzyme Activity Assay—L-Fumarate Production or Arginine Production

1. Pellet 5×10⁶ cells per sample (if you have enough cells pellet at addition 5×10⁶ cells for a no substrate control).
2. Resuspend in 20 μl of 0.1% Triton-X+protease inhibitors (0.1% Triton-X is stored in a 50 ml tube at 4° C. in molecular lab), and incubate on ice for 20 minutes with occasional vortexing.
3. Centrifuge samples at 13,000 rpm for 20 minutes at 4° C. to pellet cell debris.
4. Take 20 μl of cell lysate in to fresh eppendorfs and keep on ice.
5. Take a fresh eppendorf for each sample and add 25 μL of L-argininosuccinic acid (11.7 mM), and 55 μl PBS
6. Then add 20 ul of cell lysate to each tube. For a no enzyme control, add 20 μl of 0.1% Triton-X+protease inhibitors instead of cell lysate.
7. If performing no substrate controls do not add the ASL or ArgH substrate i.e. L-argininosuccinic acid. Instead make up to a final volume of 100 μl with SDW.
8. Incubate samples at 37° C. for 1.5 hrs. ASL or ArgH enzyme reaction will occur.
9. Make L-fumarate standards by making a 1 mM L-fumarate solution by performing a 1:4 dilution of a 4 mM L-Fumarate solution. To eppendorfs add 10, 20, 30, 50, 80 and 100 μl of L-Fumarate (1 mM), and make each standard up to 100 μl with sterile distilled water. This makes L-Fumarate concentrations of 0, 10, 20, 30, 50, 80 and 100 nM. Include a blank control.
10. To each standard add 25 μL of L-Argininosuccinic acid (11.7 mM), and 20 μl of 0.1% Triton-X+protease inhibitors. ASL enzyme reagents are added to standards to more accurately determine L-Fumarate concentrations within ASL or ArgH enzyme activity samples.
11. After 1.5 hrs at 37° C., centrifuge tubes at 13000 rpm for 1 minute to pellet any debris. In duplicate, add 50 μl of supernatant to wells of a 96-well flat bottom plate.
12. Read absorbance at 2400 nm using the microplate absorbance reader.
13. Arginine production can also be measured at Step 11 above, using HPLC or arginine ELISA according to manufacturer's instructions. For measurement of cell supernatant concentrations the protocol above can be modified accordingly, using 100 ul of cell supernatant instead of cell lysate.

### Assay to Determine Tryptophan Synthase (Trp5) Enzyme Activity Assay—Indole Depletion or Tryptophan Production

1. Pellet 5×10⁶ cells per sample (if you have enough cells pellet at addition 5×10⁶ cells for a no substrate control).
2. Resuspend in 20 μl of 0.1% Triton-X+protease inhibitors (0.1% Triton-X is stored in a 50 ml tube at 4° C. in molecular lab), and incubate on ice for 20 minutes with occasional vortexing.
3. Centrifuge samples at 13,000 rpm for 20 minutes at 4° C. to pellet cell debris.
4. Take 20 μl of cell lysate in to fresh eppendorfs and keep on ice.
5. Take a fresh Eppendorf for each sample and add 80 μl indole solution (0.005M), 400 μl DL-serine solution (0.2M), 100 μl pyridoxal phosphate solution, 20 μl glutathione (0.05M), and 120 μl of phosphate buffer (0.5M, pH7.8), 260 μl water.
6. Then add 20 ul of cell lysate to each tube. For a no enzyme control, add 20 μl of 0.1% Triton-X+protease inhibitors instead of cell lysate.
7. If performing no substrate controls do not add the Trp5 substrate i.e. indole and serine solutions. Instead make up to a final volume of 1000 μl with SDW.
8. Incubate samples at 37° C. for 1.5 hrs. Trp5 enzyme reaction will occur.
9. Make indole standards by making a 1 mM indole solution by performing a 1:4 dilution of a 4 mM L-Fumarate solution. To eppendorfs add 10, 20, 30, 50, 80 and 100 μl of Indole (1 mM), and make each standard up to 1000 μl with sterile distilled water. This makes indole concentrations of 0, 10, 20, 30, 50, 80 and 100 nM. Include a blank control. To each standard add 20 μl of 0.1% Triton-X+protease inhibitors.
10. After 1.5 hrs at 37° C., add 200 μl of 5% NaOH. Add 4 ml of toluene into each tube and centrifuge to separate the solution into 2 layers.
11. Pipette up to 1 ml of the toluene layer into separate test tubes. Add 4 ml of ethanol and 2 ml of p-dimethylaminobenzaldehyde solution (Make as follows: 36 g dissolved in 500 ml of ethanol. Add 180 ml of concentrated Hcl. When cool bring the volume to 1 L with ethanol). Allow colour change to occur for 60 minutes.
12. Read absorbance at 550 nm using the microplate absorbance reader.
13. Tryptophan production can also be measured at Step 10 above, using HPLC or tryptophan ELISA according to manufacturer's instructions. For measurement of cell supernatant concentrations the protocol above can be modified accordingly, using 100 ul of cell supernatant instead of cell lysate.

### Assay to Determine Indoleamine 2,3-Dioxygenase (IDO) Enzyme Activity Assay—Kynurenine Production

1. Pellet 5×10⁶ cells per sample (if you have enough cells pellet at addition 5×10⁶ cells for a no substrate control).
2. Resuspend in 20 μl of 0.1% Triton-X+protease inhibitors (0.1% Triton-X is stored in a 50 ml tube at 4° C. in molecular lab), and incubate on ice for 20 minutes with occasional vortexing.
3. Centrifuge samples at 13,000 rpm for 20 minutes at 4° C. to pellet cell debris.
4. Take 20 μl of cell lysate in to fresh eppendorfs and keep on ice.
5. Take a fresh Eppendorf for each sample and add 50 □l trichloroacetic acid (30%) to the cell lysates. Vortex and centrifuge at 10,000 rpm for 5 minutes.
6. Collect the supernatants and add to equal volumes of Ehrlich reagent (100 mg P-dimethylbenzaldehyde, 5 ml acetic acid).
7. Make Kynurenine standards by making a 1 mM Kynurenine solution by performing a 1:4 dilution of a 4 mM Kynurenine solution. To eppendorfs add 10, 20, 30, 50, 80 and 100 μl of Kynurenine (1 mM), and make each standard up to 1000 μl with sterile distilled water. This makes Kynurenine concentrations of 0, 10, 20, 30, 50, 80 and 100 nM. Include a blank control. To each standard add 20 μl of 0.1% Triton-X+protease inhibitors.
8. Read absorbance at 492 nm using the microplate absorbance reader.
9. Kynurenine production can also be measured at Step 4 above, using HPLC or Kynurenine ELISA according to manufacturer's instructions. For measurement of cell supernatant concentrations the protocol above can be modified accordingly, using 100 ul of cell supernatant instead of cell lysate.

### Assay to Determine Ornithine Decarboxylase (ODC1) Enzyme Activity Assay—Polyamine Production

1. Pellet 5×10⁶ cells per sample (if you have enough cells pellet at addition 5×10⁶ cells for a no substrate control).
2. Resuspend in 20 μl of 0.1% Triton-X+protease inhibitors (0.1% Triton-X is stored in a 50 ml tube at 4° C. in molecular lab), and incubate on ice for 20 minutes with occasional vortexing.
3. Centrifuge samples at 13,000 rpm for 20 minutes at 4° C. to pellet cell debris.
4. Take 20 μl of cell lysate in to fresh eppendorfs and keep on ice.
5. Polyamine production is measured, using HPLC or polyamine ELISA according to manufacturer's instructions. For measurement of cell supernatant concentrations the protocol above can be modified accordingly, using 100 ul of cell supernatant instead of cell lysate.

SEQUENCE INFORMATION
Amino acid sequence of exemplary ASS-1 enzyme domain
Sequence ID NO: 1
MSSKGSVVLAYSGGLDTSCILVWLKEQGYDVIAYLANIGQKEDFEEARKKALKLGAKKVFIEDVSREF
VEEFIWPAIQSSALYEDRYLLGTSLARPCIARKQVEIAQREGAKYVSHGATGKGNDQVRFELSCYSLA
PQIKVIAPWRMPEFYNRFKGRNDLMEYAKQHGIPIPVTPKNPWSMDENLMHISYEAGILENPKNQAPP
GLYTKTQDPAKAPNTPDILEIEFKKGVPVKVTNVKDGTTHQTSLELFMYLNEVAGKHGVGRIDIVENRF
IGMKSRGIYETPAGTILYHAHLDIEAFTMDREVRKIKQGLGLKFAELVYTGFWHSPECEFVRHCIAKSQ
ERVEGKVQVSVLKGQVYILGRESPLSLYNEELVSMNVQGDYEPTDATGFININSLRLKEYHRLQSKVT
AK
Amino acid sequence of exemplary OTC enzyme domain
Sequence ID NO: 2
MLFNLRILLNNAAFRNGHNFMVRNFRCGQPLQNKVQLKGRDLLTLKNFTGEEIKYMLWLSADLKFRIK
QKGEYLPLLQGKSLGMIFEKRSTRTRLSTETGLALLGGHPCFLTTQDIHLGVNESLTDTARVLSSMAD
AVLARVYKQSDLDTLAKEASIPIINGLSDLYHPIQILADYLTLQEHYSSLKGLTLSWIGDGNNILHSIMMS
AAKFGMHLQAATPKGYEPDASVTKLAEQYAKENGTKLLLTNDPLEAAHGGNVLITDTWISMGQEEEK
KKRLQAFQGYQVTMKTAKVAASDWTFLHCLPRKPEEVDDEVFYSPRSLVFPEAENRKWTIMAVMVS
LLTDYSPQLQKPKF
Amino acid sequence of exemplary GD2 target binding moiety
Sequence ID NO: 3
DILLTQTPLSLPVSLGDQASISCRSSQSLVHRNGNTYLHWYLQKPGQSPKLLIHKVSNRFSGVPDRFS
GSGSGTDFTLKISRVEAEDLGVYFCSQSTHVPPLTFGAGTKLELKRADAAPTVSIFPGSGGGGSGGE
VKLQQSGPSLVEPGASVMISCKASGSSFTGYNMNWVRQNIGKSLEWIGAIDPYYGGTSYNQKFKGR
ATLTVDKSSSTAYMHLKSLTSEDSAVYYCVSGMEYWGQGTSVTVSSAKTTPPSVYGRVTVSS
Amino acid sequence of exemplary CD33 target binding moiety
Sequence ID NO: 4
GSNIMLTQSPSSLAVSAGEKVTMSCKSSQSVFFSSSQKNYLAWYQQIPGQSPKLLIYWASTRESGVP
DRFTGSGSGTDFTLTISSVQSEDLAIYYCHQYLSSRTFGGGTKLEIKRGGGGSGGGGSSGGGSQVQ
LQQPGAEVVKPGASVKMSCKASGYTFTSYYIHWIKQTPGQGLEWVGVIYPGNDDISYNQKFKGKATL
TADKSSTTAYMQLSSLTSEDSAVYYCAREVRLRYFDVWGAGTTVTVSS
Amino acid sequence of exemplary mesothelin target binding moiety
Sequence ID NO: 5
MQVQLQQSGPELEKPGASVKISCKASGYSFTGYTMNWVKQSHGKSLEWIGLITPYNGASSYNQKFR
GKATLTVDKSSSTAYMDLLSLTSEDSAVYFCARGGYDGRGFDYWGQGTTVTVSSGVGGSGGGGSG
GGGSDIELTQSPAIMSASPGEKVTMTCSASSSVSYMHWYQQKSGTSPKRWIYDTSKLASGVPGRFS
GSGSGNSYSLTISSVEAEDDATYYCQQWSGYPLTFGAGTKLEIK
Amino acid sequence of exemplary EGFRVIII target binding moiety
Sequence ID NO: 6
QVQLQQSGGGLVKPGASLKLSCVTSGFTFRKFGMSWVRQTSDKRLEWVASISTGGYNTYYSDNVK
GRFTISRENAKNTLYLQMSSLKSEDTALYYCTRGYSSTSYAMDYWGQGTTVTVSSSGGGSGGGGS
GGGGSDIELTQSPASLSVATGEKVTIRCMTSTDIDDDMNWYQQKPGEPPKFLISEGNTLRPGVPSRF
SSSGTGTDFVFTIENTLSEDVGDY
Amino acid sequence of exemplary 4-1BB intracellular signalling region
Sequence ID NO: 7
KRGRKKLLYIFKQPFMRPVQTTQEEDGCSCRFPEEEEGGCEL
Amino acid sequence of exemplary OX-40 intracellular signalling region
Sequence ID NO: 8
RDQRLPPDAHKPPGGGSFRTPIQEEQADAHSTLAKI
Amino acid sequence of exemplary CD28 intracellular signalling region with
transmembrane domain
Sequence ID NO: 9
IEVMYPPPYLDNEKSNGTIIHVKGKHLCPSPLFPGPSKPFWVLVVVGGVLACYSLLVTVAFIIFWVRSK
RSRLLHSDYMNMTPRRPGPTRKHYQPYAPPRDFAAYRS
Amino acid sequence of exemplary ICOS intracellular signalling region
Sequence ID NO: 10
CWLTKKKYS SSVHDPNGEY MFMRAVNTAK KSRLTDVTL
(the cytoplasmic portion of ICOS, comprising residues 162-199 of the
full-length protein. The motif YMFM (residues 180-183 of the full-length
protein) is of particular relevance, and should be retained in an ICOS
intracellular signally region suitable for use in a protein of the invention)
Amino acid sequence of exemplary CD3 ζ intracellular signalling region
Sequence ID NO: 11
RVKFSRSADAPAYKQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
Amino acid sequence of exemplary protein of the invention GD2 ASS + OTC
Sequence ID NO: 12
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPDILLTQTPLSLPVSLGDQASISCRSSQSLVHRNGNTYLHWYLQKPGQ
SPKLLIHKVSNRFSGVPDRFSGSGSGTDFTLKISRVEAEDLGVYFCSQSTHVPPLTFGAGTKLELKRA
DAAPTVSIFPGSGGGGSGGEVKLQQSGPSLVEPGASVMISCKASGSSFTGYNMNWVRQNIGKSLE
WIGAIDPYYGGTSYNQKFKGRATLTVDKSSSTAYMHLKSLTSEDSAVYYCVSGMEYWGQGTSVTVS
SAKTTPPSVYGRVTVSSAEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKKDPKTTTPAPRPPTPAPTI
ASQPLSLRPEACRPAAGGAVHTRGLDFACDIYIWAPLAGTCGVLLLSLVITLYCKRGRKKLLYIFKQPF
MRPVQTTQEEDGCSCRFPEEEEGGCELRVKFSRSADAPAYKQGQNQLYNELNLGRREEYDVLDKR
RGRDPEMGGKPRRKNPQEGLYNELQKDKMAEAYSEIGMKGERRRGKGHDGLYQGLSTATKDTYDA
LHMQALPPRGSGATNFSLLKQAGDVEENPGPMSSKGSVVLAYSGGLDTSCILVWLKEQGYDVIAYLA
NIGQKEDFEEARKKALKLGAKKVFIEDVSREFVEEFIWPAIQSSALYEDRYLLGTSLARPCIARKQVEIA
QREGAKYVSHGATGKGNDQVRFELSCYSLAPQIKVIAPWRMPEFYNRFKGRNDLMEYAKQHGIPIPV
TPKNPWSMDENLMHISYEAGILENPKNQAPPGLYTKTQDPAKAPNTPDILEIEFKKGVPVKVTNVKDG
TTHQTSLELFMYLNEVAGKHGVGRIDIVENRFIGMKSRGIYETPAGTILYHAHLDIEAFTMDREVRKIKQ
GLGLKFAELVYTGFWHSPECEFVRHCIAKSQERVEGKVQVSVLKGQVYILGRESPLSLYNEELVSMN
VQGDYEPTDATGFININSLRLKEYHRLQSKVTAKGSGEGRGSLLTCGDVEENPGPMLFNLRILLNNAA
FRNGHNFMVRNFRCGQPLQNKVQLKGRDLLTLKNFTGEEIKYMLWLSADLKFRIKQKGEYLPLLQGK
SLGMIFEKRSTRTRLSTETGLALLGGHPCFLTTQDIHLGVNESLTDTARVLSSMADAVLARVYKQSDL
DTLAKEASIPIINGLSDLYHPIQILADYLTLQEHYSSLKGLTLSWIGDGNNILHSIMMSAAKFGMHLQAAT
PKGYEPDASVTKLAEQYAKENGTKLLLTNDPLEAAHGGNVLITDTWISMGQEEEKKKRLQAFQGYQV
TMKTAKVAASDWTFLHCLPRKPEEVDDEVFYSPRSLVFPEAENRKWTIMAVMVSLLTDYSPQLQKPK
F
Amino acid sequence of exemplary protein of the invention GD2 ASS1
Sequence ID NO: 13
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPDILLTQTPLSLPVSLGDQASISCRSSQSLVHRNGNTYLHWYLQKPGQ
SPKLLIHKVSNRFSGVPDRFSGSGSGTDFTLKISRVEAEDLGVYFCSQSTHVPPLTFGAGTKLELKRA
DAAPTVSIFPGSGGGGSGGEVKLQQSGPSLVEPGASVMISCKASGSSFTGYNMNWVRQNIGKSLE
WIGAIDPYYGGTSYNQKFKGRATLTVDKSSSTAYMHLKSLTSEDSAVYYCVSGMEYWGQGTSVTVS
SAKTTPPSVYGRVTVSSAEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKKDPKTTTPAPRPPTPAPTI
ASQPLSLRPEACRPAAGGAVHTRGLDFACDIYIWAPLAGTCGVLLLSLVITLYCKRGRKKLLYIFKQPF
MRPVQTTQEEDGCSCRFPEEEEGGCELRVKFSRSADAPAYKQGQNQLYNELNLGRREEYDVLDKR
RGRDPEMGGKPRRKNPQEGLYNELQKDKMAEAYSEIGMKGERRRGKGHDGLYQGLSTATKDTYDA
LHMQALPPRGSGATNFSLLKQAGDVEENPGPMSSKGSVVLAYSGGLDTSCILVWLKEQGYDVIAYLA
NIGQKEDFEEARKKALKLGAKKVFIEDVSREFVEEFIWPAIQSSALYEDRYLLGTSLARPCIARKQVEIA
QREGAKYVSHGATGKGNDQVRFELSCYSLAPQIKVIAPWRMPEFYNRFKGRNDLMEYAKQHGIPIPV
TPKNPWSMDENLMHISYEAGILENPKNQAPPGLYTKTQDPAKAPNTPDILEIEFKKGVPVKVTNVKDG
TTHQTSLELFMYLNEVAGKHGVGRIDIVENRFIGMKSRGIYETPAGTILYHAHLDIEAFTMDREVRKIKQ
GLGLKFAELVYTGFWHSPECEFVRHCIAKSQERVEGKVQVSVLKGQVYILGRESPLSLYNEELVSMN
VQGDYEPTDATGFININSLRLKEYHRLQSKVTAK
Amino acid sequence of exemplary protein of the invention GD2 OTC
Sequence ID NO: 14
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPDILLTQTPLSLPVSLGDQASISCRSSQSLVHRNGNTYLHWYLQKPGQ
SPKLLIHKVSNRFSGVPDRFSGSGSGTDFTLKISRVEAEDLGVYFCSQSTHVPPLTFGAGTKLELKRA
DAAPTVSIFPGSGGGGSGGEVKLQQSGPSLVEPGASVMISCKASGSSFTGYNMNWVRQNIGKSLE
WIGAIDPYYGGTSYNQKFKGRATLTVDKSSSTAYMHLKSLTSEDSAVYYCVSGMEYWGQGTSVTVS
SAKTTPPSVYGRVTVSSAEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKKDPKTTTPAPRPPTPAPTI
ASQPLSLRPEACRPAAGGAVHTRGLDFACDIYIWAPLAGTCGVLLLSLVITLYCKRGRKKLLYIFKQPF
MRPVQTTQEEDGCSCRFPEEEEGGCELRVKFSRSADAPAYKQGQNQLYNELNLGRREEYDVLDKR
RGRDPEMGGKPRRKNPQEGLYNELQKDKMAEAYSEIGMKGERRRGKGHDGLYQGLSTATKDTYDA
LHMQALPPRGSGATNFSLLKQAGDVEENPGPMLFNLRILLNNAAFRNGHNFMVRNFRCGQPLQNKV
QLKGRDLLTLKNFTGEEIKYMLWLSADLKFRIKQKGEYLPLLQGKSLGMIFEKRSTRTRLSTETGLALL
GGHPCFLTTQDIHLGVNESLTDTARVLSSMADAVLARVYKQSDLDTLAKEASIPIINGLSDLYHPIQILA
DYLTLQEHYSSLKGLTLSWIGDGNNILHSIMMSAAKFGMHLQAATPKGYEPDASVTKLAEQYAKENG
TKLLLTNDPLEAAHGGNVLITDTWISMGQEEEKKKRLQAFQGYQVTMKTAKVAASDWTFLHCLPRKP
EEVDDEVFYSPRSLVFPEAENRKWTIMAVMVSLLTDYSPQLQKPKF
Amino acid sequence of exemplary protein of the invention Mesothelin
ASS1 + OTC
Sequence ID NO: 15
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPMQVQLQQSGPELEKPGASVKISCKASGYSFTGYTMNWVKQSHGKSL
EWIGLITPYNGASSYNQKFRGKATLTVDKSSSTAYMDLLSLTSEDSAVYFCARGGYDGRGFDYWGQ
GTTVTVSSGVGGSGGGGSGGGGSDIELTQSPAIMSASPGEKVTMTCSASSSVSYMHWYQQKSGTS
PKRWIYDTSKLASGVPGRFSGSGSGNSYSLTISSVEAEDDATYYCQQWSGYPLTFGAGTKLEIKTTT
PAPRPPTPAPTIASQPLSLRPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQ
GLSTATKDTYDALHMQALPPRGSGATNFSLLKQAGDVEENPGPMSSKGSVVLAYSGGLDTSCILVW
LKEQGYDVIAYLANIGQKEDFEEARKKALKLGAKKVFIEDVSREFVEEFIWPAIQSSALYEDRYLLGTSL
ARPCIARKQVEIAQREGAKYVSHGATGKGNDQVRFELSCYSLAPQIKVIAPWRMPEFYNRFKGRNDL
MEYAKQHGIPIPVTPKNPWSMDENLMHISYEAGILENPKNQAPPGLYTKTQDPAKAPNTPDILEIEFKK
GVPVKVTNVKDGTTHQTSLELFMYLNEVAGKHGVGRIDIVENRFIGMKSRGIYETPAGTILYHAHLDIE
AFTMDREVRKIKQGLGLKFAELVYTGFWHSPECEFVRHCIAKSQERVEGKVQVSVLKGQVYILGRES
PLSLYNEELVSMNVQGDYEPTDATGFININSLRLKEYHRLQSKVTAKGSGEGRGSLLTCGDVEENPG
PMLFNLRILLNNAAFRNGHNFMVRNFRCGQPLQNKVQLKGRDLLTLKNFTGEEIKYMLWLSADLKFRI
KQKGEYLPLLQGKSLGMIFEKRSTRTRLSTETGLALLGGHPCFLTTQDIHLGVNESLTDTARVLSSMA
DAVLARVYKQSDLDTLAKEASIPIINGLSDLYHPIQILADYLTLQEHYSSLKGLTLSWIGDGNNILHSIMM
SAAKFGMHLQAATPKGYEPDASVTKLAEQYAKENGTKLLLTNDPLEAAHGGNVLITDTWISMGQEEE
KKKRLQAFQGYQVTMKTAKVAASDWTFLHCLPRKPEEVDDEVFYSPRSLVFPEAENRKWTIMAVMV
SLLTDYSPQLQKPKF
Amino acid sequence of exemplary protein of the invention Mesothelin ASS
Sequence ID NO: 16
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPMQVQLQQSGPELEKPGASVKISCKASGYSFTGYTMNWVKQSHGKSL
EWIGLITPYNGASSYNQKFRGKATLTVDKSSSTAYMDLLSLTSEDSAVYFCARGGYDGRGFDYWGQ
GTTVTVSSGVGGSGGGGSGGGGSDIELTQSPAIMSASPGEKVTMTCSASSSVSYMHWYQQKSGTS
PKRWIYDTSKLASGVPGRFSGSGSGNSYSLTISSVEAEDDATYYCQQWSGYPLTFGAGTKLEIKTTT
PAPRPPTPAPTIASQPLSLRPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQ
GLSTATKDTYDALHMQALPPRGSGATNFSLLKQAGDVEENPGPMSSKGSVVLAYSGGLDTSCILVW
LKEQGYDVIAYLANIGQKEDFEEARKKALKLGAKKVFIEDVSREFVEEFIWPAIQSSALYEDRYLLGTSL
ARPCIARKQVEIAQREGAKYVSHGATGKGNDQVRFELSCYSLAPQIKVIAPWRMPEFYNRFKGRNDL
MEYAKQHGIPIPVTPKNPWSMDENLMHISYEAGILENPKNQAPPGLYTKTQDPAKAPNTPDILEIEFKK
GVPVKVTNVKDGTTHQTSLELFMYLNEVAGKHGVGRIDIVENRFIGMKSRGIYETPAGTILYHAHLDIE
AFTMDREVRKIKQGLGLKFAELVYTGFWHSPECEFVRHCIAKSQERVEGKVQVSVLKGQVYILGRES
PLSLYNEELVSMNVQGDYEPTDATGFININSLRLKEYHRLQSKVTAK
Amino acid sequence of exemplary protein of the invention Mesothelin OTC
Sequence ID NO: 17
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPMQVQLQQSGPELEKPGASVKISCKASGYSFTGYTMNWVKQSHGKSL
EWIGLITPYNGASSYNQKFRGKATLTVDKSSSTAYMDLLSLTSEDSAVYFCARGGYDGRGFDYWGQ
GTTVTVSSGVGGSGGGGSGGGGSDIELTQSPAIMSASPGEKVTMTCSASSSVSYMHWYQQKSGTS
PKRWIYDTSKLASGVPGRFSGSGSGNSYSLTISSVEAEDDATYYCQQWSGYPLTFGAGTKLEIKTTT
PAPRPPTPAPTIASQPLSLRPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQ
GLSTATKDTYDALHMQALPPRGSGATNFSLLKQAGDVEENPGPMLFNLRILLNNAAFRNGHNFMVR
NFRCGQPLQNKVQLKGRDLLTLKNFTGEEIKYMLWLSADLKFRIKQKGEYLPLLQGKSLGMIFEKRST
RTRLSTETGLALLGGHPCFLTTQDIHLGVNESLTDTARVLSSMADAVLARVYKQSDLDTLAKEASIPIIN
GLSDLYHPIQILADYLTLQEHYSSLKGLTLSWIGDGNNILHSIMMSAAKFGMHLQAATPKGYEPDASVT
KLAEQYAKENGTKLLLTNDPLEAAHGGNVLITDTWISMGQEEEKKKRLQAFQGYQVTMKTAKVAASD
WTFLHCLPRKPEEVDDEVFYSPRSLVFPEAENRKWTIMAVMVSLLTDYSPQLQKPKF
Amino acid sequence of exemplary protein of the invention CD33 ASS + OTC
Sequence ID NO: 18
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPGSNIMLTQSPSSLAVSAGEKVTMSCKSSQSVFFSSSQKNYLAWYQQI
PGQSPKLLIYWASTRESGVPDRFTGSGSGTDFTLTISSVQSEDLAIYYCHQYLSSRTFGGGTKLEIKR
GGGGSGGGGSSGGGSQVQLQQPGAEVVKPGASVKMSCKASGYTFTSYYIHWIKQTPGQGLEWVG
VIYPGNDDISYNQKFKGKATLTADKSSTTAYMQLSSLTSEDSAVYYCAREVRLRYFDVWGAGTTVTV
SSTTTPAPRPPTPAPTIASQPLSLRPEACRPAAGGAVHTRGLDFACDIYIWAPLAGTCGVLLLSLVITLY
CKRGRKKLLYIFKQPFMRPVQTTQEEDGCSCRFPEEEEGGCELRVKFSRSADAPAYKQGQNQLYNE
LNLGRREEYDVLDKRRGRDPEMGGKPRRKNPQEGLYNELQKDKMAEAYSEIGMKGERRRGKGHD
GLYQGLSTATKDTYDALHMQALPPRGSGATNFSLLKQAGDVEENPGPMSSKGSVVLAYSGGLDTSC
ILVWLKEQGYDVIAYLANIGQKEDFEEARKKALKLGAKKVFIEDVSREFVEEFIWPAIQSSALYEDRYLL
GTSLARPCIARKQVEIAQREGAKYVSHGATGKGNDQVRFELSCYSLAPQIKVIAPWRMPEFYNRFKG
RNDLMEYAKQHGIPIPVTPKNPWSMDENLMHISYEAGILENPKNQAPPGLYTKTQDPAKAPNTPDILEI
EFKKGVPVKVTNVKDGTTHQTSLELFMYLNEVAGKHGVGRIDIVENRFIGMKSRGIYETPAGTILYHAH
LDIEAFTMDREVRKIKQGLGLKFAELVYTGFWHSPECEFVRHCIAKSQERVEGKVQVSVLKGQVYILG
RESPLSLYNEELVSMNVQGDYEPTDATGFININSLRLKEYHRLQSKVTAKGSGEGRGSLLTCGDVEE
NPGPMLFNLRILLNNAAFRNGHNFMVRNFRCGQPLQNKVQLKGRDLLTLKNFTGEEIKYMLWLSADL
KFRIKQKGEYLPLLQGKSLGMIFEKRSTRTRLSTETGLALLGGHPCFLTTQDIHLGVNESLTDTARVLS
SMADAVLARVYKQSDLDTLAKEASIPIINGLSDLYHPIQILADYLTLQEHYSSLKGLTLSWIGDGNNILHS
IMMSAAKFGMHLQAATPKGYEPDASVTKLAEQYAKENGTKLLLTNDPLEAAHGGNVLITDTWISMGQ
EEEKKKRLQAFQGYQVTMKTAKVAASDWTFLHCLPRKPEEVDDEVFYSPRSLVFPEAENRKWTIMA
VMVSLLTDYSPQLQKPKF
Amino acid sequence of exemplary protein of the invention CD33 ASS1
Sequence ID NO: 19
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPGSNIMLTQSPSSLAVSAGEKVTMSCKSSQSVFFSSSQKNYLAWYQQI
PGQSPKLLIYWASTRESGVPDRFTGSGSGTDFTLTISSVQSEDLAIYYCHQYLSSRTFGGGTKLEIKR
GGGGSGGGGSSGGGSQVQLQQPGAEVVKPGASVKMSCKASGYTFTSYYIHWIKQTPGQGLEWVG
VIYPGNDDISYNQKFKGKATLTADKSSTTAYMQLSSLTSEDSAVYYCAREVRLRYFDVWGAGTTVTV
SSTTTPAPRPPTPAPTIASQPLSLRPEACRPAAGGAVHTRGLDFACDIYIWAPLAGTCGVLLLSLVITLY
CKRGRKKLLYIFKQPFMRPVQTTQEEDGCSCRFPEEEEGGCELRVKFSRSADAPAYKQGQNQLYNE
LNLGRREEYDVLDKRRGRDPEMGGKPRRKNPQEGLYNELQKDKMAEAYSEIGMKGERRRGKGHD
GLYQGLSTATKDTYDALHMQALPPRGSGATNFSLLKQAGDVEENPGPMSSKGSVVLAYSGGLDTSC
ILVWLKEQGYDVIAYLANIGQKEDFEEARKKALKLGAKKVFIEDVSREFVEEFIWPAIQSSALYEDRYLL
GTSLARPCIARKQVEIAQREGAKYVSHGATGKGNDQVRFELSCYSLAPQIKVIAPWRMPEFYNRFKG
RNDLMEYAKQHGIPIPVTPKNPWSMDENLMHISYEAGILENPKNQAPPGLYTKTQDPAKAPNTPDILEI
EFKKGVPVKVTNVKDGTTHQTSLELFMYLNEVAGKHGVGRIDIVENRFIGMKSRGIYETPAGTILYHAH
LDIEAFTMDREVRKIKQGLGLKFAELVYTGFWHSPECEFVRHCIAKSQERVEGKVQVSVLKGQVYILG
RESPLSLYNEELVSMNVQGDYEPTDATGFININSLRLKEYHRLQSKVTAK
Amino acid sequence of exemplary protein of the invention CD33 OTC
Sequence ID NO: 20
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPGSNIMLTQSPSSLAVSAGEKVTMSCKSSQSVFFSSSQKNYLAWYQQI
PGQSPKLLIYWASTRESGVPDRFTGSGSGTDFTLTISSVQSEDLAIYYCHQYLSSRTFGGGTKLEIKR
GGGGSGGGGSSGGGSQVQLQQPGAEVVKPGASVKMSCKASGYTFTSYYIHWIKQTPGQGLEWVG
VIYPGNDDISYNQKFKGKATLTADKSSTTAYMQLSSLTSEDSAVYYCAREVRLRYFDVWGAGTTVTV
SSTTTPAPRPPTPAPTIASQPLSLRPEACRPAAGGAVHTRGLDFACDIYIWAPLAGTCGVLLLSLVITLY
CKRGRKKLLYIFKQPFMRPVQTTQEEDGCSCRFPEEEEGGCELRVKFSRSADAPAYKQGQNQLYNE
LNLGRREEYDVLDKRRGRDPEMGGKPRRKNPQEGLYNELQKDKMAEAYSEIGMKGERRRGKGHD
GLYQGLSTATKDTYDALHMQALPPRGSGATNFSLLKQAGDVEENPGPMLFNLRILLNNAAFRNGHNF
MVRNFRCGQPLQNKVQLKGRDLLTLKNFTGEEIKYMLWLSADLKFRIKQKGEYLPLLQGKSLGMIFEK
RSTRTRLSTETGLALLGGHPCFLTTQDIHLGVNESLTDTARVLSSMADAVLARVYKQSDLDTLAKEASI
PIINGLSDLYHPIQILADYLTLQEHYSSLKGLTLSWIGDGNNILHSIMMSAAKFGMHLQAATPKGYEPDA
SVTKLAEQYAKENGTKLLLTNDPLEAAHGGNVLITDTWISMGQEEEKKKRLQAFQGYQVTMKTAKVA
ASDWTFLHCLPRKPEEVDDEVFYSPRSLVFPEAENRKWTIMAVMVSLLTDYSPQLQKPKF
Amino acid sequence of exemplary protein of the invention EGFR
ASS1 + OTC
Sequence ID NO: 21
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPQVQLQQSGGGLVKPGASLKLSCVTSGFTFRKFGMSWVRQTSDKRLE
WVASISTGGYNTYYSDNVKGRFTISRENAKNTLYLQMSSLKSEDTALYYCTRGYSSTSYAMDYWGQ
GTTVTVSSSGGGSGGGGSGGGGSDIELTQSPASLSVATGEKVTIRCMTSTDIDDDMNWYQQKPGEP
PKFLISEGNTLRGVPSRFSSSGTGTDFVFTIENTLSEDVGDYYCLQSFNVPLTFGDGTKLEKALEQKLI
SEEDLAKPTTTPAPRPPTPAPTIASQPLSLRPEACRPAAGGAVHTRGLDFACDIYIWAPLAGTCGVLLL
SLVITLYCKRGRKKLLYIFKQPFMRPVQTTQEEDGCSCRFPEEEEGGCELRVKFSRSADAPAYKQGQ
NQLYNELNLGRREEYDVLDKRRGRDPEMGGKPRRKNPQEGLYNELQKDKMAEAYSEIGMKGERRR
GKGHDGLYQGLSTATKDTYDALHMQALPPRGSGATNFSLLKQAGDVEENPGPMSSKGSVVLAYSG
GLDTSCILVWLKEQGYDVIAYLANIGQKEDFEEARKKALKLGAKKVFIEDVSREFVEEFIWPAIQSSALY
EDRYLLGTSLARPCIARKQVEIAQREGAKYVSHGATGKGNDQVRFELSCYSLAPQIKVIAPWRMPEFY
NRFKGRNDLMEYAKQHGIPIPVTPKNPWSMDENLMHISYEAGILENPKNQAPPGLYTKTQDPAKAPN
TPDILEIEFKKGVPVKVTNVKDGTTHQTSLELFMYLNEVAGKHGVGRIDIVENRFIGMKSRGIYETPAG
TILYHAHLDIEAFTMDREVRKIKQGLGLKFAELVYTGFWHSPECEFVRHCIAKSQERVEGKVQVSVLK
GQVYILGRESPLSLYNEELVSMNVQGDYEPTDATGFININSLRLKEYHRLQSKVTAKGSGEGRGSLLT
CGDVEENPGPMLFNLRILLNNAAFRNGHNFMVRNFRCGQPLQNKVQLKGRDLLTLKNFTGEEIKYML
WLSADLKFRIKQKGEYLPLLQGKSLGMIFEKRSTRTRLSTETGLALLGGHPCFLTTQDIHLGVNESLTD
TARVLSSMADAVLARVYKQSDLDTLAKEASIPIINGLSDLYHPIQILADYLTLQEHYSSLKGLTLSWIGD
GNNILHSIMMSAAKFGMHLQAATPKGYEPDASVTKLAEQYAKENGTKLLLTNDPLEAAHGGNVLITDT
WISMGQEEEKKKRLQAFQGYQVTMKTAKVAASDWTFLHCLPRKPEEVDDEVFYSPRSLVFPEAENR
KWTIMAVMVSLLTDYSPQLQKPKF
Amino acid sequence of exemplary protein of the invention EGFR ASS1
Sequence ID NO: 22
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPQVQLQQSGGGLVKPGASLKLSCVTSGFTFRKFGMSWVRQTSDKRLE
WVASISTGGYNTYYSDNVKGRFTISRENAKNTLYLQMSSLKSEDTALYYCTRGYSSTSYAMDYWGQ
GTTVTVSSSGGGSGGGGSGGGGSDIELTQSPASLSVATGEKVTIRCMTSTDIDDDMNWYQQKPGEP
PKFLISEGNTLRGVPSRFSSSGTGTDFVFTIENTLSEDVGDYYCLQSFNVPLTFGDGTKLEKALEQKLI
SEEDLAKPTTTPAPRPPTPAPTIASQPLSLRPEACRPAAGGAVHTRGLDFACDIYIWAPLAGTCGVLLL
SLVITLYCKRGRKKLLYIFKQPFMRPVQTTQEEDGCSCRFPEEEEGGCELRVKFSRSADAPAYKQGQ
NQLYNELNLGRREEYDVLDKRRGRDPEMGGKPRRKNPQEGLYNELQKDKMAEAYSEIGMKGERRR
GKGHDGLYQGLSTATKDTYDALHMQALPPRGSGATNFSLLKQAGDVEENPGPMSSKGSVVLAYSG
GLDTSCILVWLKEQGYDVIAYLANIGQKEDFEEARKKALKLGAKKVFIEDVSREFVEEFIWPAIQSSALY
EDRYLLGTSLARPCIARKQVEIAQREGAKYVSHGATGKGNDQVRFELSCYSLAPQIKVIAPWRMPEFY
NRFKGRNDLMEYAKQHGIPIPVTPKNPWSMDENLMHISYEAGILENPKNQAPPGLYTKTQDPAKAPN
TPDILEIEFKKGVPVKVTNVKDGTTHQTSLELFMYLNEVAGKHGVGRIDIVENRFIGMKSRGIYETPAG
TILYHAHLDIEAFTMDREVRKIKQGLGLKFAELVYTGFWHSPECEFVRHCIAKSQERVEGKVQVSVLK
GQVYILGRESPLSLYNEELVSMNVQGDYEPTDATGFININSLRLKEYHRLQSKVTAK
Amino acid sequence of exemplary protein of the invention EGFR OTC
Sequence ID NO: 23
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPQVQLQQSGGGLVKPGASLKLSCVTSGFTFRKFGMSWVRQTSDKRLE
WVASISTGGYNTYYSDNVKGRFTISRENAKNTLYLQMSSLKSEDTALYYCTRGYSSTSYAMDYWGQ
GTTVTVSSSGGGSGGGGSGGGGSDIELTQSPASLSVATGEKVTIRCMTSTDIDDDMNWYQQKPGEP
PKFLISEGNTLRGVPSRFSSSGTGTDFVFTIENTLSEDVGDYYCLQSFNVPLTFGDGTKLEKALEQKLI
SEEDLAKPTTTPAPRPPTPAPTIASQPLSLRPEACRPAAGGAVHTRGLDFACDIYIWAPLAGTCGVLLL
SLVITLYCKRGRKKLLYIFKQPFMRPVQTTQEEDGCSCRFPEEEEGGCELRVKFSRSADAPAYKQGQ
NQLYNELNLGRREEYDVLDKRRGRDPEMGGKPRRKNPQEGLYNELQKDKMAEAYSEIGMKGERRR
GKGHDGLYQGLSTATKDTYDALHMQALPPRGSGATNFSLLKQAGDVEENPGPMLFNLRILLNNAAFR
NGHNFMVRNFRCGQPLQNKVQLKGRDLLTLKNFTGEEIKYMLWLSADLKFRIKQKGEYLPLLQGKSL
GMIFEKRSTRTRLSTETGLALLGGHPCFLTTQDIHLGVNESLTDTARVLSSMADAVLARVYKQSDLDT
LAKEASIPIINGLSDLYHPIQILADYLTLQEHYSSLKGLTLSWIGDGNNILHSIMMSAAKFGMHLQAATPK
GYEPDASVTKLAEQYAKENGTKLLLTNDPLEAAHGGNVLITDTWISMGQEEEKKKRLQAFQGYQVTM
KTAKVAASDWTFLHCLPRKPEEVDDEVFYSPRSLVFPEAENRKWTIMAVMVSLLTDYSPQLQKPKF
DNA sequence encoding ASS1
Sequence ID NO: 24
atgtccagcaaaggctccgtggttctggcctacagtggcggcctggacacctcgtgcatcctcgtgtggctgaaggaacaaggctatgacgtc
attgcctatctggccaacattggccagaaggaagacttcgaggaagccaggaagaaggcactgaaActtggggccaaaaaggtgttcattg
aggatgtcagcagggagtttgtggaggagttcatctggccggccatccagtccagcgcactgtatgaggaccgctacctcctgggcacctctct
tgccaggccctgcatcgcccgcaaacaagtggaaatcgcccagcgggagggggccaagtatgtgtcccacggcgccacaggaaagggg
aacgatcaggtccggtttgagctcagctgctactcactggccccccagataaaggtcattgctccctggaggatgcctgaattctacaaccggtt
caagggccgcaatgacctgatggagtacgcaaagcaacacgggattcccatcccggtcactcccaagaacccgtggagcatggatgaga
acctcatgcacatcagctacgaggctggaatcctggagaaccccaagaaccaagcgcctccaggtctctacacgaagacccaggaccca
gccaaagcccccaacacccctgacattctcgagatcgagttcaaaaaaggggtccctgtgaaggtgaccaacgtcaaggatggcaccacc
caccagacctccttggagctcttcatgtacctgaacgaagtcgcgggcaagcatggcgtgggccgtattgacatcgtggagaaccgcttcattg
gaatgaagtcccgaggtatctacgagaccccagcaggcaccatcctttaccatgctcatttagacatcgaggccttcaccatggaccgggaa
gtgcgcaaaatcaaacaaggcctgggcttgaaatttgctgagctggtgtataccggtttctggcacagccctgagtgtgaatttgtccgccactg
catcgccaagtcccaggagcgagtggaagggaaagtgcaggtgtccgtcctcaagggccaggtgtacatcctcggccgggagtccccact
gtctctctacaatgaggagctggtgagcatgaacgtgcagggtgattatgagccaactgatgccaccgggttcatcaacatcaattccctcagg
ctgaaggaatatcatcgtctccagagcaaggtcactgccaaa
DNA sequence encoding OTC
Sequence ID NO: 25
Atgctgtttaatctgaggatcctgttaaacaatgcagcttttagaaatggtcacaacttcatggttcgaaattttcggtgtggacaaccactacaaa
ataaagtgcagctgaagggccgtgaccttctcactctaaaaaactttaccggagaagaaattaaatatatgctatggctatcagcagatctgaa
atttaggataaaacagaaaggagagtatttgcctttattgcaagggaagtccttaggcatgatttttgagaaaagaagtactcgaacaagattgt
ctacagaaacaggcttagcacttctgggaggacatccttgttttcttaccacacaagatattcatttgggtgtgaatgaaagtctcacggacacgg
cccgtgtattgtctagcatggcagatgcagtattggctcgagtgtataaacaatcagatttggacaccctggctaaagaagcatccatcccaatt
atcaatgggctgtcagatttgtaccatcctatccagatcctggctgattacctcacgctccaggaacactatagctctctgaaaggtcttaccctca
gctggatcggggatgggaacaatatcctgcactccatcatgatgagcgcagcgaaattcggaatgcaccttcaggcagctactccaaagggt
tatgagccggatgctagtgtaaccaagttggcagagcagtatgccaaagagaatggtaccaagctgttgctgacaaatgatccattggaagc
agcgcatggaggcaatgtattaattacagacacttggataagcatgggacaagaagaggagaagaaaaagcggctccaggctttccaagg
ttaccaggttacaatgaagactgctaaagttgctgcctctgactggacatttttacactgcttgcccagaaagccagaagaagtggatgatgaag
tcttttattctcctcgatcactagtgttcccagaggcagaaaacagaaagtggacaatcatggctgtcatggtgtccctgctgacagattactcacc
tcagctccagaagcctaaattt
DNA sequence encoding GD2 scFv
Sequence ID NO: 26
GATATTCTGCTCACACAGACCCCACTCTCCCTGCCCGTGTCACTCGGGGATCAGGCTAGCATTT
CTTGCCGCTCATCTCAGTCTCTGGTCCACCGGAATGGGAACACATACCTCCATTGGTACCTCCA
GAAACCTGGACAGAGCCCTAAACTGCTCATCCACAAAGTCTCAAATCGGTTCTCCGGCGTGCCC
GATCGCTTTAGCGGATCCGGATCTGGGACCGACTTCACACTGAAAATCTCACGAGTGGAGGCTG
AGGATCTCGGCGTCTACTTCTGTAGTCAGAGTACCCACGTCCCACCCCTCACCTTTGGCGCTGG
AACAAAACTGGAGCTGAAACGAGCCGATGCTGCTCCTACCGTGTCCATCTTTCCTGGCTCCGGG
GGAGGCGGGAGCGGAGGCGAAGTGAAACTCCAGCAGTCTGGCCCTTCTCTCGTGGAACCTGGC
GCTTCTGTGATGATCTCCTGTAAGGCCTCTGGATCTTCCTTTACCGGCTACAACATGAACTGGGT
CCGGCAGAACATTGGCAAATCCCTGGAATGGATTGGCGCCATCGATCCTTACTACGGCGGCACA
TCATACAATCAGAAATTCAAGGGGCGAGCAACACTCACTGTCGACAAATCTTCATCCACCGCCTA
CATGCACCTGAAATCTCTCACATCCGAGGATAGTGCTGTCTACTACTGTGTCTCTGGCATGGAAT
ACTGGGGACAGGGAACTTCTGTCACCGTGTCTAGTGCCAAAACCACACCTCCCTCCGTGTACGG
ACGAGTCACTGTCTCATCT
DNA sequence encoding CD33 scFv
Sequence ID NO: 27
Ggatccaacatcatgctgacccagagccctagcagcctggccgtgtctgccggcgagaaagtgaccatgagctgcaagagcagccagag
cgtgttcttcagcagctcccagaagaactacctagcctggtatcagcagatcccaggccagagccctaagctgctgatctactgggccagcac
cagagaaagcggcgtgcccgatagattcaccggaagcggttctggcaccgacttcaccctgacaatcagcagcgtgcagagcgaggacct
ggccatctactactgccaccagtacctgagcagccggacctttggcggaggcaccaagctggaaatcaagagaggcggcggaggctcag
gcggaggcggatctagtggcggaggatctcaggtgcagctgcagcagccaggcgccgaggtcgtgaaacctggcgcctctgtgaagatgt
cctgcaaggccagcggctacaccttcaccagctactacatccactggatcaagcagacccctggacagggcctggaatgggtgggagtgat
ctaccccggcaacgacgacatcagctacaaccagaagttcaagggcaaggccaccctgaccgccgacaagtctagcaccaccgcctaca
tgcagctgtccagcctgaccagcgaggacagcgccgtgtactactgcgccagagaagtgcggctgcggtacttcgatgtgtggggagccgg
caccaccgtgaccgtgtcatct
DNA sequence encoding Mesothelin scFv
Sequence ID NO: 28
Atgcaggtacaactgcagcagtctgggcctgagctggagaagcctggcgcttcagtgaagatatcctgcaaggcttctggttactcattcactg
gctacaccatgaactgggtgaagcagagccatggaaagagccttgagtggattggacttattactccttacaatggtgcttctagctacaacca
gaagttcaggggcaaggccacattaactgtagacaagtcatccagcacagcctacatggacctcctcagtctgacatctgaagactctgcagt
ctatttctgtgcaagggggggttacgacgggaggggttttgactactggggccaagggaccacggtcaccgtctcctcaggtgtaggcggttca
ggcggcggtggctctggcggtggcggatcggacatcgagctcactcagtctccagcaatcatgtctgcatctccaggggagaaggtcaccat
gacctgcagtgccagctcaagtgtaagttacatgcactggtaccagcagaagtcaggcacctcccccaaaagatggatttatgacacatcca
aactggcttctggagtcccaggtcgcttcagtggcagtgggtctggaaactcttactctctcacaatcagcagcgtggaggctgaagatgatgc
aacttattactgccagcagtggagtggttaccctctcacgttcggtgctgggacaaagttggaaataaaa
DNA sequence encoding EGFRvIII scFv
Sequence ID NO: 29
Caggtacaactccagcagtctgggggaggcttagtgaagcctggagcgtctctgaaactctcctgtgtaacctctggattcactttcagaaaattt
ggcatgtcttgggttcgccagactagtgacaagaggctggaatgggtcgcaTccattagtactggcggttataacacgtactattcagacaatg
taaagggccgattcaccatctccagagagaatgccaagaacaccctgtacctgcaaatgagtagtctgaagtctgaggacacggccttgtatt
actgtacaagaGgctattctagtacctcttatgctatggactactggggccaagggaccacggtcaccgtctcctcaagtggaggcggttcagg
cggaggtggctctggcggtggcggatcggacatcgagctcactcagtctccagcatccctgtccGtggctacaggagaaaaagtcactatca
gatgcatgaccagcactgatattgatgatgatatgaactggtaccagcagaagccaggggaaccccctaagttccttatttcagaaggcaata
ctcttcggccgggagtcccatcccGattttccagcagtggcactggcacagattttgtttttacaattgaaaacacactctcggaagatgttggag
attactactgtttgcaaagctttaacgtgcctcttacattcggtgatggcaccaagcttgaaaaagctctagagcagaaactgatctcggaagaa
gatctggcgaagccc
DNA sequence encoding ASL
Sequence ID NO: 30
GACGCCATCCCGGCCAGAAAAGCCCTGGCCAGTGGCGGGCGCGACACTATCCGTGCGGCCAG
GCGGAGGTGAGTGCGCGGCGGCCGGATGGGCGGGACGGGCGTGGAGGACGCCGAGCACCGT
GGCGCGCGCTCACGTCCGCGTCCCCAAGGGCTGCGCTCCCTCAAGCGCAGTGCCCAGAACTC
GGAGCCAGCCCGGCCCGGGGGACCCTGCTGGCCAAGGAGGTCGTCAGTCCGGTCTTGTCTTC
CAGACCCGGAGGACCGAAGCTTCCGGACGACGAGGAACCGCCCAACATGGCCTCGGAGGTGA
GTGGGACCTCGGGGACTCCGGTCCTCCTAGCCTCCAAAGGAGAGAGTGGGGGCGCCAGACCT
GCCTCGGGCCACCCTGCTGGGAATCGCCCTCCAGGAAGCAATTTTGAAAATTACCTAGGAAGCC
TGCACCCCCAGCCCTCCCGGGCGCATCATCTGGAGCCCAGCAGTCACCTTTACCAGGACTCAC
CAGTATCCGCAGGCAGCCCTTGTGGCAAACCCACCAACCCACACTACTAGGGGTAGAGTGGCTC
TGCCCTCACCTCACAGTGATGCCTGCCTGGCCAGGAAAAGTGGCTCCCAAGCCTTCAGCCTTCC
AACTCTTCCTTCCTTCTTACCACGTGTCCTCCTGTCAGGTCCCACCCCACACCACATCCCTTCTC
CTGCTAGAGCAATTGTCCCTGTTTATAGAATAAAGCTCAGCCCCTAAGTGTTCTTGTCCTTGACTG
TGGCATGTGGAAAGAGCCAGGAAAAAGGGGACGTCGCCTCGTGGCTCCAGCAACCCTGGTGCC
TGGTCCCTTCCTGTCTCACTGGACCCTGCCTCTTAGGGTCAGTGGCTCCTGGCCTCTCCTCCTG
ACCACTGAGATGCTGGATTCCCAGGCAGAGGTTTTCCTTCCTTGGGCCATAGTTGATTTATCTGG
CAATGGGGGTAATAATAGCTGTCGGCCTCACTCTGTAAGGCACTAGATTATGAGGCCATTGCTTT
GGACCCTTCAGGTGAGAGGGGCTGTTCGCCTGATGCTTGATGAAGGGAACTCCGGGAAGCAGG
AGGTCTGGGTTCCAGGCCCTTTTGGCCTTCATTAGCTAGCAATTCACTTCCTCTTTCTCAATGCC
CTGCAAGCTCAGTGCCCTGCAAGCTTCTGGGTCATGGCAGGGGGGTAGGGCCTGGACTTTGGA
GCCAAACAGACTTGGTTTCTGTACCAGTCACTTGAGCCCTTTAAACCTCTTTCCTCATTTGTGAAA
TAGGGGTAATATTGCCCACCTCATAAAAGGCCGTAATAACATATGTGAAATCCCTAGCA
CAGGGCTGAGCAACAGTAGGTGCTCAATAAATGGTGGCTAACCACAACAATACTGATATTTCTAC
TTTGGGAGGCCGAGGCGGGAGGACTGCTGAAGCCCAGGAATTCCAGACCAGCCAGGGCAATGT
AATGAGACCCTGTCTCTACAAAAAGATTTTGAAAATTACCCAGGTGTGGTGGCGTACACCTGTAG
TCTCACAGGAGGCGGAGGCATGAGGATTGCTTGAGCCCAGGAGTTTGAGACTGCAGTGAGCAT
GATCTCACCACTGCACTCCAGCCCAGGCAACAGAGTGAGATCCAGTTTCAAAAAAATAAAAATAA
AAAAACCTGGCAGGCATGGTGGCTCACACCTGTAATCCCAGCACTTTGGGAGGTCGAGGTGGG
CAGATCACCTGAGGTCAGGAGTTTGAGACCAGCCTGGCCAACATGGTGAAACCCCGTCTCTACT
AAAAATGCAAAAATTAGCTGGGTGTAGTGGTGGGTGCCTGTAGTCCCAGCTATTCGGGAGGCTG
AGGCAGGAGAATCGCTTGAACTCGGGAGGCGGAGGTTGCAGTGAGCTGAGATCGCACCACTGC
ACTCCAGCCTGGGCGACAAAGCAAGACTCCACCTCAAAAAAAGAAAAAAAACCCACAAGTCCCA
AAACCAAAACTGGTATTTCCCATGTACATTCGACCTTAACTGTTGCTCATTCAACCCAGCCCAACT
CAGTGCCCCATCCCCTGGCCCTGAAGAGACCATTCTGGCCCAGATGTGTCCTGGCTTGGAGTAG
CACCTTCTGCTACCACACTAGGCCTCCACTCTCCTCAGTGCCCAAGGGGAGGCACCTCACTCTG
ATCTCCCTGTGGGGTCCTCTTATGCCTGCCAGTAAGGATAGGAGTTTGGTTCTAGAGCAGAGTG
GTCTGGCTTCCAACAAGCCCAGTGTTCTAGGTGACCTCAGGTTGCCCCAGGCCTTCCTGTAGGT
TGGCACTAATTGGTTTGGCTGCAGCTCCACTTATTAAGTAGTTATTTTTATTACTAACAACCTAGG
CAGGGTGGCCCAGGAGCCCGCTGGGGGAGGCGGTGCCAGGCTCCTGGCTGAGGGGCAGGCT
GGCCCCAATTCTGACTGGCTGGCTCCAGTGATCAGGACCAGGGCCCCACGTGGTGCTTTGCTG
GAGATCTAGGCTTGGTGGGGCAGCTGTGGGGTAAGGGGCAGGACCAGCTCAAAGATGGGGTG
GGGGCGGAGGCTGCCTCTGCAGGGCAGAGTCCTTTGGCAGTCGGGACTGTTGGGCATAGAGG
AGTCAGCTCACAGCTCAGGGCCAGAGCACTGGAGAGGTCTCTGGGGTGCATACAGGAACCAGG
AGTGGAGCTGAAGCATGTCCTATCCCCTGCCAGCCCTCCCTTAGTAACAGCTGGCATTTCTCGA
GTCCTTCCTGAGCACCAGGCACCGTGTTATGTATGCAATTTGCAAATATTATCTGATTAAATGCTC
ACAATAAGGCTATCAGAGAGGTACTATTATTATCCTTATCTTATTATTATTATTATTATTATTTTTAA
GAAGGAGTCTTGCTCTGTCGCCCAAGCTGGAGTACAATGGCGCCATCTCTGCTCACCGCAACCT
CTACCTCCAGGATTCAAGTGATTCTCCTGCTTCAGACTCCCAAGTGGCTGGGACTACAGCTTCCT
GCCACCGCGCCTGGCTAATGTTTATATTTTTAGTACAGATGAGGTTTCACCATGTTGGCCAGGCT
GATCTCGAACTCCTGACCTCAAGCGATCCACCTGCCTGGGCCTCCCAACCCTGAGGGTTTTTGT
TTTTGTTTTTGTTTTGAGACAGAGTCTTGCTCTGTCGCCCAGGCTGGAGTGCAGTGGCACGATCT
CAGCTCACTGCAAGCTCCGCCTCCCGGGTTCACGCCATTCTCCTGCCTCAGCCTCCCAAGTAGC
TGGGACTACAGGCGCCCAACACCCCCATGCCTGGCTAATTTTTTTGTGTGTGTTTTCAGTAGAGA
CGGAGTTTCACCATGTTAGCCAGGATGGTCTCCATCTCCTGACCTCGTGATCTGCCCACCTCAG
CCTCCCAAAGTGCTAGGATTATAGGTGTGAGCCACCACGCCCAGCCCCTGAGGTTTAATAATAG
GTGCCAGGCCAGGTGGTTAATAGAAGTCTGGGGCATTGTAGGGGGACAGAGGAGGATATATGT
CCCCATTGGCCATTGTAGACTCCCTTCCACAAAAAGGACGTCAGTGAAGTGACATGCCCACCTCT
ACCCCACCCTCCTCCCAGTCCTGGGCACTAGGGCTGCTCCCCAGGTGTTCTGTACCCCCTCCCC
ACTCTGTCCCATGCCCTGGCCTCTGCCCTCTTTCAAAACATAGATGTGGCTGGCGCCTAGGCTC
ATGCCTATAATCTCAGCACTTTGGGAAGCTGAGGCTGGAGGACAGCTTGAGCCCAGGAATTCAA
GACCAGCCTGGGCAACATAGTGAGACCCTGTCTCTACCAATTATTTTATTTTATTTTATTTTGTTCA
TTTATTTATTTATTTTGAGACAGAGTTTTGCTCTGTCACCTAGGCTGGAGTGCAGTGGCGTGATCT
TGGCTCACTGCAACCTCCGCCTCCCGGGTTCAAGCGATTCTCCTGCCTCAGCCTCCTAAGTAGC
TGGAACTACAGGCGAGTGCCACCACGCCTGGCTAATTTTTGTATTTTTAGTAGAGACCAGGTTTC
ACCATGTTGACCAGGATGGTCTCTGTCTCCTGACCTCATGATCCACCCACCTTAGCCTTCCAAAA
TGCTGGGATTACAGGCATGAGCCACCACTCCCAGTCCTATAAAATTTTAAAAAAATGTCTGGGTG
TGGTGGCGCATGCTTGTAGTCCCAACTATTGGGGAGGCTGAGGCAAGAGGATTGGTTGAGACCA
GGAGTTTGAGGCTGCAGTGAGCTATGATGGTGCCACCGCACTCCAACCTGGGTAACAAAGTGAG
ACCCTGTGTCTAAAAAAGAATTTAAAGGCCGGGTGTGGTGGCTCACACCCGTAATCCCAGGACT
TTGGGAGGCCGAGGTGGGCAGATCACGAGGTCAGATTAAGACCATCCTGGCTAACAAGGTGAA
ACCACGTCTCTACTGAAAAAAAAAAATACAAAAAATTAGCCAGGCATGGTGGTGGGCACCTGTAG
TCCCAGCTACTCAGGAGGCTGAGGCAGGAGAATGGCGTGAATCTGGGAGGTGGAGCTTGCAGTGA
GCCAAGATCCTGCCACTGCACTCCAGCCTGGGTGACAAAGAGAGACTCCATCTCAAAAAAAAAA
AAAAAAAAAAAGAATTAAAAAAGATTTTTTTAATGAACAAAACAGGCTCGGCACAGTGGCTCATGC
CTGTAATCCCAAGCACTTCGGGATGCCAAGGTCAGGGGATCACCTGAGATCAGGAGTTCGAGAC
CAGCCTGACCAACATGGTGAAACCCCGTCTCTACTAAAAATATAAAACTCAGCCAGGTGTGGTGG
CACACGCCTGAAATTCCAGCTACTCGGG
AGGCTGAGGCAGGAAAATTGGCTTGAAGCCGGGAGGTGGAGGTTGCAGTGAGCCGAGATCACG
CCACTGCACTCCAGCCTAGGCAACAGAGTGAGACTCTATCTCAAAGAAAACGAACAAAACATAGA
TGCCTACATACCATTCCTCTGCCCTTGGCTCCTGGGGAGTAAGGGATCACCCAGTGACCTCCTA
GAAGGCCAGTGACAATGGGGGGTGTCAGGGTGCTTTTCAGAGCCAAGGGAGTGGTAGGAATTG
GGATCTTAGTCCAGCTCCAAGCTGTGAGGGAGAGAGTTGCAGGGCACTTAAGCTTGGTGGAGAC
CCTCAAGGCCTCTTTGCCTGTCCCTGCAGCAAAGGTTCTGGACACCAGAGCCAAGTCCAGAAGC
CCTGGTGGAACAGGGGTGAAAAGCATAGGTTCTGACTTCAGACTGCTGGGCCGCAGCCCTGGC
TATCCCACCCCAGGTGAGAGCAGGCTGCTCTGTGCCTCAGTTTCCCCATCTTCATAGTGGAATTG
TATTGGTGCCTACCCAGAGGGTTGTGTCAACAATTAGGATGGCACCTAGCACCTTGGTCAGTGG
TGGGAAAGGTTCCAGAAGTTCTGCTGTGGTCCCAGGGGTGTCTCAGGCCCTGCCATCATCTCCT
TGGAGGGGTGCCATGTGGTGGGAAAGAACCCCAACTTCAAGGCCACACACAGTGGCTCATGCC
TGTAATCCTAGCACTTTCAGAGGCCAAGATGGGAGGATCACCTGAGGTCAGGAGTTCAAGACCA
GCCTGGCCAACATGGTGAAACCCCATCTCTACTAATGATACAAAAATTAGCTGGGTGTGGTGGCA
CGTGCCTGTAATCCCAGCTACTTGGGAGTCTGAGGCAGGAGAATCTCTTGAACCTGGGAGGCAG
AGTTTGCAGTGAGCTGAGATGGCACCACTGTACTCCAGCCTGGCCGACAAAGTGAGACTCTGTC
TCAAAAAAAAAAAAAAAGAACCCAAACTTTTGGTGTTCAGCCATGTTCCCATGCTCACTCCCAGG
GTGGTGACTCTGGGAAGGTCTCAGCCTCCTTGTCTGCCCAGTTAGAATGATCTGATGCCCCTGC
TACCATCAGACTTGATAAGTTTCCCAAAGACTCTTTGCAAGAAGCACTGTTCTGGAGGGTGGAGG
AGAGACTAATTGTTCTTGCTCTCCTGGCCAGAGTGGGAAGCTTTGGGGTGGCCGGTTTGTGGGT
GCAGTGGACCCCATCATGGAGAAGTTCAACGCGTCCATTGCCTACGACCGGCACCTTTGGGAG
GTGGATGTTCAAGGCAGCAAAGCCTACAGCAGGGGCCTGGAGAAGGCAGGGCTCCTCACCAAG
GCCGAGATGGACCAGATACTCCATGGCCTAGACAAGGTACTTGCCGTGGCCCAAGCCCCACCC
AAGGCCCCTTCCCTGTGGCCCCAGGCTCCCACCAAATCCCTGAGCAAACAGTGCAGTGTTGCCC
ATCTGTGGTTTCACATTGAACTAATTATATACTCAAGTGCTGTTTAACTGTGTGCCTTGATGACTG
CCTCTCTCCATCCTTTAATGACCCCTGTGGCCCACATGGCTCATGGGTAAAGGTGTGCTGGGCC
TGAGATGCCCCCTCCCAGGGTGCGCTTCCAGGACTCAGCTCCTGGGCAGGGACAGTCAGTCAC
CAGGGATAGGGTGGGACCAAGGCAGGGGCTCTCTTGGCTGCTGATGCCTGCTCACCTGACCCC
GGCATTGCTGCTACCCACTACAGGTGGCTGAGGAGTGGGCCCAGGGCACCTTCAAACTGAACT
CCAATGATGAGGACATCCACACAGCCAATGAGCGCCGCCTGAAGGTACGACCCCTGGAGCCCC
ACCGCTTTCCTTGCCTCCCCTCTCCACCTTGCCCAGGGCCACTTTGAGCATTAGCACCATTCTGT
TTACTTCGCCATTGGCAGACAGCATGTGAGACCTCAGGACATGAGCCAGGCACCCTGGCTCATG
CCTATAATCCTAGCACTTTGGGAGGCTGAGGTGGGAGGATTGCTTGAGACCAGGAGTTCGAGAC
CAGCCTGGGCAACATAATGAGGTCCCACAGCTACAAAAATTAAAAAAAGAAAAGAAAAAAAGAAC
AGGCCTCAGCAGAAATGGCGAGAGATTTGGGGAGGACCCGGAGCCCTGGGGTATGGAGGTAG
GTTGGCAGGGCTGATGAGGAAAACTGCCCTGCCTGGGTTGACTCCTCTGGGGGTATAGACCGT
GACCCTGGGTCTCCCTTCACCTCCAGGAGCTCATTGGTGCAACGGCAGGGAAGCTGCACACGG
GACGGAGCCGGAATGACCAGGTGCTTTAGCCCCTCCACCCCCTGCTCCGTGTTGTCCCAACCTT
GAGGAGCCCAGGGGGCAGTTAGAGTTCTGCAGCGGTCCTGGCTCCTCAGGGAAGCAACACATC
GGCCTCCCTGAGCACCATCTCCTCCTTGCACAGGTGGTCACAGACCTCAGGCTGTGGATGCGG
CAGACCTGCTCCACGCTCTCGGGCCTCCTCTGGGAGCTCATTAGGACCATGGTGGATCGGGCA
GAGGCGTGAGTCCTACAGGGACACCCAGGGGGCAGACAGAGGTGTGATGGAAGCCTGAACAG
GAGACCTAGGGGGCAGGGGTGAACAGCGTGGGGGTGCCAGGCCCTGGGGGACAGGGGCATC
CCAGAACTCCAGGATCGAGGCAGAGCAGCCAGGAGTGGGCCATTTCCTGCAGGCCCCAATACT
CCCATGCCAGTCTAGCTCAGCAGGCAGAGAAGACTAACCCTTCGTGGGGCTGGGTGCGGTGGC
TCACGCCTGTAATCTCAGCACTTTGGGAGGCCGAGGTGGGTGGATCACCTGAGGTCAGGAGTTC
GAGACCAGCCTGGCCAACATGGGAAAACTCTGTCTCTACTAAAAATACAAAAATTAGGCAATGTG
GTGGTGTGCGCCTGTAATCCCAGCTACTCGGGAGCCTGAGGCAGAAGAACTGCTTGAACCCGG
GAGGAGGAGGTTGCAATGAGCCGAGATCGCGCCATTGCACTCCAGCCTGGGCTACAGAGCGAG
ACTCCTGTCTCAAAAAAAAAGAAAAAAAAAAAAGAAAACTCACCATTTGCAGATTTGAAGGCAGGA
AGCTAAGCCAAGCACAGCTAGCTTGGCTGTGCCTGGAGCAGCCAGAGTCACTCCCCACACTGC
CTGTCCCCCAGATCCCCCATCCTAAGCTTCGCCTCCCCATCCAGCCCATCTGGCAAAAGACAGA
GCCAAAGGCTGCCTCCTGCTGGCCTCATTTCAGGCTTTGGCTTCTGGGACCTGGTGTCTTTGGG
ACTGGATTTGTTCCTTGCAGACCTGGACGAAGAGCTGCTGAGAAGTCTCCATGTGTTGTCAGAG
ACCCCTCCTCTTCCTCAACTCCCTGTGACCCCTGTTGTGCAGACTTGGGGGAAAACAAGGGCAC
AAGAATTGTCACCCAGCAGGTGGTGTGGGGCTGCTAGGAGGAACAGGGAGTGTCTGCTACTGA
GTTCAGGGTTTCTTTAATTTTTTGTTGTTGTTGTTTGTTGTTGTTTTTTTTTTTTTGAGACAGGGTTT
TGCTCTGTCACCTAGTCTGGAGTGTAGTGGCGCTATCTGAGCTAACTGCAAACTCTGCCTCCTG
GGTTCAAGTGATTCTAGTGCCTCAGCCTCCCAAGTAGCTGAGATTACAGGTGTGCACCACCATGT
CCAGCTAATTTTTGTATTTTTTTCAGTAGAGATGGGTTTTGCCATGTTGACCAGGCTGGTCTTGAA
CTCCTGAGCTCAGGTGATCTGCCCGCGTCGGCCTGCCAAAGTGCTAGGATTACACCCATAAGCC
ACTGCGCTCAGCTTAATTTTTAAATTTTTAACTTTTTAAATTGTCTTTAGAGATGAGATCCTGCTCT
GTCACCTAGGCTGGAGTGCAATGGCTTGGTAATAGCTCACTGCAGTCTCAAACTCCTGGACTCA
AATGATCCTCCCACCTCAGCTTTCTGAGTAGCTAGGACCACAGGTGTGCACCACCTGTGAGACA
GAGTCTTGCTCTGTTGCCCAGGCTGGAGTGCAGTGGCGTGATCTCCACTCACTGCAACCTCTGC
CTCCCAGGTTCACGCCATTCTCCTGCCTCAGCCTCCCGAGT
AGCTGGGAGTACAGGTGCCCACCACCACGCCCGGCTAATTTTTTGTATTTTTAGTAGAGACGGG
GTTTCACCATGTTAGCCAGGATGGTCTCAATCTCCCGACCTCATGATCCACCCACCTCGGACTCT
CAAAGTGCTGGGATTACAGGTGTGAGCCACCGTGCCCAGCCGCGAATTCTTTAAATTTTTTGTAG
AAACAGGGTCTCACTATGTGGCTCAGGCTGGTCTCAAACTCCCGGCCTTAAGTGATCCTTCCCTC
TTGGCCTCCCAAAGTGCTGGGATTAAAGACTTGAGCCACCGTGCCTGGCCTTGAGTACAGAATT
TCTTCATGGGGTGATGAAAATGTTCTAAAATTGGTTGTGGTGATGGTTGTACAGTAAAGTGTAAAC
TTTAAATGAGTAAATTGTGAATGATATCTCAGTAAAGCTGGTTTATTTAAAACAACAGGCCAGGTG
CTGTGGCTCACGCCTGTAATCCCAGCACTTTGGAAGGCTGAGGCGGGTGAATCACCTGAGGTCA
AGAGTTCGAGACCAGCCTGGCCAACATGGTGAAACCCCATCTCTACTAAAAATACACAAAATTAG
CTGGGTGTGATGGTGGGCACCTGTAGTCCCAGCTACTTGGGAGGCAGGAGAATCTCTTGGACCT
GGGAGGTGGAGGTTGTAGTGAGCCGAGATCACGCCACTGCATTCCAGCCTGGGCAACAAGAGC
GAAACTCTTTCTCAAAAACAACAACAACAAAAAAACAGGCCAGGTATGGTGGCTCATATCTGTAAT
CCCAGCCCTTTGGGAGGCCAAGGCAGGAGGACTGCCTGAAACCAGGAGTTTCAGACCACTCTG
GGCAACATAGCAAGACCCCATCTTTTTTTTTTTTTTTGAGACGGAGTCTCGCTCTGTCGCCCAGG
CTGAAGTGCAATGGTGCAATCTCAACTCACTGCAAGCTCTGCCTCCTGGGTTCATGCCATTCTCC
TGCCTCAGCCCTCCTGAGTAGCTGGAACTACAGGCGCCCACCACTACGCCCGGCTAATTTTTTG
TATTTTTAGTATAGATGGGGTTTCACCGTGTTAGCCAGGATGGTCTCGATCTCCTGACCTTGTGA
TCTGCCCGCCTCGGCCTCCCAAAGTGCTGGGATTACAGGCGTGAGTTACCGCGCCTGGCCACA
AGACCCCATCTTTACAAAAAACTAAAAATTAGCTGGGCATGGTGGCATGTCCCTTTAGTCCCAGC
TACTCAGGAGGCTGAGACAGGAGGATCGCTTGAGCCCAGGAGATCGAGGCAGCAGTGAGCTAT
GATCATGCCACTGCACTCCAGTCTGGGCAACAGAACGAGACCTTGTCTCTAAAAATAAAAACAAA
ACAAAACAACAAGAAAACAGGACCATCACTCACAGCACCTCTGCCTCTGCCCTGCCTACTTGAAT
GAGGTGCAGGGCATCTCACCTGCTCAGAGCAGCCCTTGAATGAGCCCCAGCTATTTCTAGGGTC
CTCAAACGAAACCTCCCACGGCCAAGTCATACCCAACATGGGCCTCCTCCCCTATTCTGGCCCC
TGCTCGGAGATGCTGAGTGACAGAGGCTGGACTTGGGGTGTTTCTGGCAAAGCCTCACTGCAG
GAAGCCCCACAGCTCAGGCCCAGTCCTTGGTTCACACGGTCCCACTTCCAGCTTCTTTTGCCCT
TAAGACTGATTTGTCCCTGGGAGATCACCAGATCCCTCATTCAGGTGGAGTGCTGCAGCGTGAC
ACTTTTTCCAGGGGTGACCCAGGCCTGCAGGGTTCCAGTGTCACAGGCAGGCCTTGCATGAGC
CTCCACCCGAGCTTCTGCTCCTCCTCTCCCACAGGGAACGTGATGTTCTCTTCCCGGGGTACAC
CCATTTGCAGAGGGCCCAGCCCATCCGCTGGAGCCACTGGATTCTGAGGTGAGCCAGGTGAGG
TGCAGGGGCTGTGCTAGAGGGGAGGACCCCGGCTGCCCTGACCCTCCTGCCCCTGGCTTCCCA
CAGCCACGCCGTGGCACTGACCCGAGACTCTGAGCGGCTGCTGGAGGTGCGGAAGCGGATCA
ATGTCCTGCCCCTGGGGAGGTGGGTGAGGCTCCAGTGCCCCGAGGGCCTGGTGGGGGTGGCT
GCTGCATAGCCTTAGGGATTGACAGAGCTGGGAAGTGCAGAGTGGGACAGAAAACCGCCTTATC
TGCTCAGCGGGGGACTCTGCATGGAGCCCCAGCTCTCGCTAAGGTGACGACCAAGCCATTGAA
TGTGTCTGAGCAGGGCCAGAGCCCTCCAGCAAGGCTCCTGGCAAGCCCAGCCTGCTGCCCTCA
GCCTGACATGTGGGAACATGTGTCAGGAGACAAGTGTCCTGCACCCAGGGTGACTTAGTGCTTG
GGGACAAGTGTTTTGTGGACACTTGGGGACAAGTATTCTGTACCCAAGGAGACTGGGCCAGGGA
AGAGGCTAAGCGCCAGGTGGTTGCCCTGGCAACCAGGACTTGGTTCTCTGTGTGTGCGTTCGTG
TGTGTGTGTGTGTGTGTGTGTGTGTGTGTGTGTGTGTCAGGGCTGCCTGCCAGGAGCCCTGGTC
ACCATGAATCCCTGTCCCTGCAGTGGGGCCATTGCAGGCAATCCCCTGGGTGTGGACCGAGAG
CTGCTCCGAGCAGGTGAGACGTCCTGCCCCTCCTCCCCAGGGAGAATCACCCTCAGCACCCGC
CAAGACCTGCAGACACACCTGAAACCAGAGGGCAGGGGCCTGTGGCTCCTGGTGAAACCTTCA
TTCATTGCCTATGGGCACTGAGGTCATCAAGTTCAGGGGTCACTCATGGCAGGGATGCCTGGTA
CTGAGAGACTCAGGGCTCCTGCCTCCCTCCTGGGACTGTGCAAAAGATCCCTCCCCCCAGCTGT
TGCCCCACCCTGATCAGGGGAGGGGGCTGGGCAACCTAGTTGGGGGAGAGGGGGCCACTCCC
TGTCCTCCAGCTTAGCCCTGCTTCCTCCCACCCCCCCAGAACTCAACTTTGGGGCCATCACTCTC
AACAGCATGGATGCCACTAGTGAGCGGGACTTTGTGGGTGAGTCCTGGGGAGCCAGTCCCCTG
CCCTGTGCCTCACTTTAGTCCTTCAGCCCAGCTTCTCTCCAGTTTCCTCCCACACCTCCACGGAC
AGGCTGGTTGTGGTGATATTGTACACTGAAGTATAAACCTTAAATGGGTAAAGTGGGTGGGGCAT
GGTGGTTCACCATGCCCAGCACTGGCCAACATGGTGAAACCCCATCTCTACTAAGAATACAAAAT
TTAGCTGGGTGTGTGGTGGCAGGTGCCTGTAATCCCAGCTACTCAGGAGTTCTGAGGCCAGAGA
ATCACTTGAACCCAGGAGGCGGAGGCTGCAGTGAGCCAAGATCACGCCAGTGCACTCCAGCCT
GGGCAACAAGAGCGAAACTCCATCTCAAAAAATAAAATAAAATAAAATAAAAATAAATAGGCCAG
GCATGGTGGCTCACGCCCGTAATCCTAGCACTTTGGGAGTCCGAGGCAGGTGGATCACATGAG
GTCAGGAGTTTAAGACCAGCCTGGCCAACATGGTGAAACCCCATCTCTACTAAAAGCACAAAAAT
TAGCTGGGCATGGTGGTGCATGCCTGTAATCCCAGCTACTCGGGAGGCTAAGGAAGGAGATTC
GCTGGAACCTGGGAGGTAGAGGTTGCAGTGAGCCAAGATTGTGCCACTGTACTCCAGCCTGTG
CATTGGGAGCGAGACTCCATCTCAATAAATAAATAAATAAATAAATGGATAAATTGTATGTGAGTG
ATAACTCAGTAAAGCTGGTTTATTTAAAACAACAACAATAACAAAAAACACGCTAGGTGCAATGGC
TTACGTTTGTAATCCTAGCACTTTGGGAGGCCAAAGCAGAAGGATTGCTTGAGCCCACAAGTTTC
AGAACAGCTTGGGCGACATAGCACGACCCCATCTTTGCGAAAAATGAAAATTTAGCCGGGTCCC
CCCACCGCCTAACCTCCTCCTGCCCCCTGTATGGTCAGGCTGGGTGGGGATGGGAGAGGCCTG
GTGACTGGGAACCTTTTCTCCCAGCCGAGTTCCTGTTCTGGGCTTCGCTGTGCATGACCCATCT
CAGCAGGATGGCCGAGGACCTCATCCTCTACTGCACCAAGGAATTCAGCTTCGTGCAGCTCTCA
GATGCCTACAGGTAAGCCCTGAACTGCCACCTCCATCTGCCGCTGCCGGCCTCTGTATCCCCCG
CCGCCCGCGGACGTGGCTGCCTTCCTCCCCGTCCCACCCCTCCGCCAGACCTGGCCATTGCGG
CGCTGGACCAGCCAAGGGTCCAGCCCCTTCAGCGCCAGCACCTCTGTCCCCAGCACGGGAAGC
AGCCTGATGCCCCAGAAGAAAAACCCCGACAGTTTGGAGCTGATCCGGAGCAAGGCTGGGCGT
GTGTTTGGGCGGGTGAGCAAGGCAGGGGGAGGGGCGGGGCCTCTGGGCTGATGGTGGGTGG
CCAGGGGGGCAGGATCCCGGGTCCAGCCCCTGTGCCTCCCTCTTCCCGCAGTGTGCCGGGCT
CCTGATGACCCTCAAGGGACTTCCCAGCACCTACAACAAAGACTTACAGGTGCGAGGCCGGGG
GAGGCCTGGCTAGTACGTGCCAGTTCTCAGGGCTCTGGCACACTCAGGCAGGGCCCCACCCCG
GGATTGCCATACATCCTCCCATCCTGTGCACACAGCTCCATCCGTGGCTGCCCTTGAACTCTCTG
CCCTTCCTTTGTTGGGGTATTGAGTGTTCTTCCCATGGAAGGCAGTGGGGATGCCTCAGTGGGG
GGGTGGGGCTGTGGGGACCCTGGGTGCCAGGGGGCTGCTAGGCCCTCACCTCCTGCCATGTG
CCTCCCAGGAGGACAAGGAAGCTGTGTTTGAAGTGTCAGACACTATGAGTGCCGTGCTCCAGGT
GGCCACTGGCGTCATCTCTACGCTGCAGGCAAGACATCACCCCCCTGCTTCTCCTCCCCTAGGT
CCCAGGCACTGGGGTGGGCATGCGGGGAGGGTGGCCTTGGGAGGAGGTGAGGTGGGGCTGG
AGGACCTGGGGCAGGGAAGGAGAGGTGTGCTCGCTCCTGCTCCTGGGGAACAGGGAAAGGAC
AGAAACTGCTGCCATGCAGTGGAAGTAGATGAGACTCAGGGGGCCTGGGGCCTGTCAAATGGC
CTGACCAGAACTCTTTAAAAAAAGAAAATCTAAACAAAAGGCCAGGTGCAGTGGCTCATGCCTGG
AATCTCACACTTTGGGAGGCCGAGGCAGATGGAGCACTTGAGGTCAGGGGTTTGAGACCAGCC
TGGCCAACATGGCGTAACCACGTCTCTACTAAAAATACAAAAATTAGCCAGGCGTGATGGCCCAC
ACCTGTAATCCCAGCTACTCAGGAGGCGGAGGCAGAAGAATAGCTTGAACCCAGGAGATGGAA
GTTGTAGTGAGCCAAGATCATGCCGCTGCACTCCAGCCTGGACCACAGAGTAAAACTCCATCTA
CAAATATATAAATTAAATTAAATTAAATTAAATATCTTTAAAAAACATTTTTTAGAGACAGGGTCACT
CTCTGTCGCCCAGGCTGGAGTGCAGTGGTGCGGTCGTAGCTCACTGCAGCCTCAAACTCTTGG
GCTCAAGTGATCTTCCCACCTCAGTCTCCAGAGTAGCTGGGACTACAAACATGCGCCACCACGC
CTGGCTAATTTTTTTATTTTTTGTAGAGACAGGGTCTCCCTATGTTTCCCAGGCTGGTCTCAAATT
CCTGGCCCCAAGCCATCCTCCCACCTTGGTCTCCCAAGGTGCTGGGATTATAGGCATGAGCCAC
TTTGCCTGGCTGATTTCTTTTAAAATCAATTATTATGGGAAATTTATGTATATAACAGCTAGAGAAT
GCATAATGAACCCTATGTACCGACACCCAGCTTCAATGATAATCAACTCACGGACATCCTGGCTC
CAGCTGTCTTTACCCACAGCTCTCTCCCACTCCCTTACCCCCTTATTTTGAAGCAAATTCCCATCA
TCACATCATTTCATTCCTAAATAGTTCAGGATATGTCTTGAAATCAGTGTTTCTTGGCTGGGTGCA
GAGCCTCATGCCTGTAATCCCATCAATTTGCGAGACTAAGGTGGGCAGATCACTCGAGGTCAGG
AGTTCGAGACCAGCCTGGCCAACATGGCGAAACCCCGTCTTTACTAAAAATATAAAAATTAGCTG
GGTGTGGTGGTACACGCCTGTAATCCCAGCTACTCAGGAGGCTGAGGCAGGAGAATTGCTTGAA
CCCGGGAGATGGAGACTGCAGTGAGCAGAGATCACGCCACTGCGCTCCAGCCTGTGTGACAGT
GCAAGACTCCATCTCAAAAAAAAAAAAAAAAAAAAAAGGCAGTGTTTCTGGAGGCTAGTCCCCCA
ACTAGCAGCACCAGCATCACCTCAGAAGTCCTGAGAAATGTGATGTGAGGCCCCACTCCAGATG
GCTGAATCAGAGACTCTGGGGGTGCTCCCCAGCAATTTGTATTTTTCTTAGTAAATTCTCCAGTG
GCTAGGCCTGGTGGCTCATGCTTGTAATCCCAGCACTTTGAGAAGCTGAGGCAGGAGAAGCGCT
TGAGCCCAGGAGTTCAAAACCAGCCTGAGCAACATAGCGAGACCTTGTCTGTAAAATTAAAAAAA
TTAAATTAGCCAGTCGTGATGGCGTGTACCTGTGGTCCCAGCCACTTAGGAGACTGATGTGGGA
GGATCCCTTGAGCCCAGGAGCTCAAGGATGCAGAGAGCCAGGATTGTGCCATTGCACTCCAACA
TGGGCGACCCTGTCTCAAAAAAGCCCAAAACAACAACAACAAATTAGCTAGGCACGGTGGTGTG
CATGGCTGTAGTCCCAGCTACTTGGGAGGCTGAGGCTGGAAGATCCCTTGAGTCCAGGCTGCA
GAGGGCTATAATGGCCACTGCACTCCAGCCTGGACAACAGAGCAAGACCCTGTCTCCTAAAACA
GAAAACAAATCCTCCAGGAACATCTGATGCATGCTGAAGATAAGGACTCTTTGAAAACATAAAGG
CCAGTAAAACATACAGGCCAGTAAGTGTTCATAGCACATGTAAATATTATCGATAATTATGAGAAG
ATGGTTCAAGTTGAGAGTGAGACAGAGCCGAGTGGGTAAGAGAGTATCTGCCCAAGGCAGGGA
TGTCCTGGCAGAGGGGCAGGTCCTGGGCCTGGCAGCTTCAGATCCCAGGGTCCCCAGGGCTCA
CCACTCGCCCACCTGTGCCCCCAGATTCACCAAGAGAACATGGGACAGGCTCTCAGCCCCGAC
ATGCTGGCCACTGACCTTGCCTATTACCTGGTCCGCAAAGGGGTAAGTGTGTAGCAGCCAGGGG
GAGGGTGAGGAGATGGGGTGCCCCCCCCAGAGGGTGGGGGAGCTCAGGAATGGGTGCAAGCG
GCCCAGCCTGGTGGCTCACCCCTGTAATCCCAGCACTTTGGGAAGCCGAGGTGGGCGGGTCAC
TTGAGGCCAGGAGTTCGAGACCAGCCTGGTCAACATGGTGAAACCCCGTCTCTTTTGATGTAAA
AATACAACAATTAGCTGGGTGTGGTGGCACACTCCTGTAATCCCAGTTACTCGGGAGGCTGAGG
CAGGAGAATTGATTGAACTGGGAGGTGGAGTTTGCGGTGAGGTGAGATCGCGCCACTGCACTC
CAGCCTGGGCAACAGAGCGAGACTTTGTGTCAAAAAGAAAAAAAAAAAAAAAAGGAAGGGGGTG
CAGGCAATGGAGGCAGATCAGGGCATGGAGAAACCTGCCTCAGCGCCATCTTCCTCCCTGGCA
CCCAGATGCCATTCCGCCAGGCCCACGAGGCCTCCGGGAAAGCTGTGTTCATGGCCGAGACCA
AGGGGGTCGCCCTCAACCAGCTGTCACTGCAGGAGCTGCAGACCATCAGGTACGGCCCATCCC
CTTCCCCATGCTGCCTCCTAGGAAGTGAGCCTGGGTGCCTGGAGCCCAGGGTGGCCTGGCGCC
CTGGCCCACCTCTTCCTCTCTCCCCAGCCCCCTGTTCTCGGGCGACGTGATCTGCGTGTGGGAC
TACGGGCACAGTGTGGAGCAGTATGGTGCCCTGGGCGGCACTGCGCGCTCCAGCGTCGACTG
GCAGATCCGCCAGGTGCGGGCGCTACTGCAGGCACAGCAGGCCTAGGTCCTCCCACACCTGCC
CCCTAATAAAGTGGGCGCGAGAGGAGGCTGCTGTGTGTTTCCTGCCCCAGCCTGGCTCCCTCG
TTGCTGGGCTTTCGGGGCTGGCCAGTGGGGACAGTCAGGGACTGGAGAGGCAGGGCAGGGTG
GCCTGTAATCCCAGCACTTTGGAAGGGCAAGGTGCGAGGATGCTTGAGGCCAGGAGTTTGACA
CAGCCTGGGCAACACAGGGAGACCCCCATCTCTACTCAATAATAAAACAAATAGCCTGGCGTGG
TGGCCCATGCATATAGTCCCAGCTACTTGTAAGGCTGAGGTGAGAGGACACTTGTGCCCAGGAG
TGGAGGCTGCAGTGAGCTATGATCACGCCACTGCATTCCAGCCTGGATAACAGAGTGAGAACCT
ATCTCTAAAAATAAATAAATAAACGAAAAATAAA
DNA sequence encoding ODC1
Sequence ID NO: 31
GAAGCCGGGGGCGGGGGCCACGCGTGGGGCAGGCGGTGCTCGGCTCGGCTGACGTCGGCCCGCCGGCGCC
CCACCAGCTCCGCGCGGGCCCGGGTTGGCCACCGCCGGGCCCCCGCCCCTCCCCCGGCGGTGTCCCGGCC
GGAACCGATCGTGGCTGGTTTGAGCTGGTGCGTCTCCATGGCGACCCGCCGGTGCTATAAGTAGGGAGCG
GCGTGCCGTGGGGCTTTGTCAGTCCCTCCTGTAGCCGCCGCCGCCGCCGCCCGCCGCCCCTCTGCCAGCA
GCTCCGGCGCCACCTCGGGCCGGCGTCTCCGGCGGGCGGGAGCCAGGCGCTGACGGGCGCGGCGGGGGCG
GCCGAGCGCTCCTGCGGCTGCGACTCAGGCTCCGGCGTCTGCGCTTCCCCATGGGGCTGGCCTGCGGCGC
CTGGGCGCTCTGAGGTGAGGGACTCCCCGGCCGCGGAGGAAGGGAGGGAGCGAGGGCGGGAGCCGGGGCG
GGCTGCGGGCCCCGGGCCCCGGGCACGTGTGCGGCGCGCCTCGCCGGCCTGCGGAGACACGTGGTCGCCG
AGCGGGCCACGACCTTGAGGCGCCGCTTCCTCCCGGCCCGGGGTTCTCCCGCGGCTGGATAAGGGTGATC
CGGGCGCCTCGTTCTGCCCCCGTCTTCACAGCTCGGGGCTGGAGGGGCCTAGGGGAGACCCACCCGGAGA
CCCTGCGGCCCCGCGCCGGCCTCTTTCCCAACCCTTCGGCGGCCGCGCGCTGGCCGGGGAGCCGTTGGGG
AGGCCCTGGCGGCCGCGCAGCAGGTGCAGGGGCGCAGAGCCCGGGCTCGCCTTGGTACAGACGAGCGGGC
CCCGGCCTTGGCGCCTTCAGTTTCCTTCCAGTTTTTATTTTCGCTGTGTCTACAGAGCAGATGACACCAA
TTTGGAAACCCGCGAGAGTGGGTAGAGCTAAGATAGTCTTGCTGTAGTAGCTGTGATATTAGATGCTCGG
CCATGACTTAGAGGTGTTTATTTAAGGACTGTGAATGACTCGGTGATTTCGGAAAAGCTTGGCTTAGATG
AACGGACATACACAGGGGAGACAGCCCTAAGGTTTGCAGAAAAGGCTGATTGTGCTGTTTGCGAAGTCGA
AATAATTGGTGAAAGTGTAGAAGGCAGAACCTCTCAGGAATGTCTGGGGAGGACAAAGAATGTGTTGGCT
GACTTTGTTTAAACATAAAATTGGGCAGACTTTAATTGATTTGTGAAATTTTTTTCAAAGTTTGTTTGAA
TTAGCCCCTATCTCTTCTAACATTATCCTCTTGTGCTAATTGATTGACCATTTTAAATAACTTAGCTGTT
ACAGAAAGACCGAAAGGTGTTCTTCAGTAAAATATATTCAAGTAAGTTACTTAAGTAACGCCTTAAAAGA
TACAGAAAAGCAAAAAAGTATTGGCGTATTAAAAAGAAATCAAAACTTTCCAAGTTTAGGCCTGAACATT
GCCTTAAAAATATTTAATAAGGCCTCAAATGACCCAGTCCGAGACTGCATGAGCCTATTTATTATTAAAT
TGTAAATATTCTTCATATAAACAAAAATATATAACCATGTCTGTAACAAAAATGGTTTTGCTAGCGTTGT
TACTCTCTTCCCTTCTCCGAGGGGTGATTTAGGCAACTTCGGAGGTTGACAATGCCAAGCAGTCACAATA
GATAGAGCTTTAAAGCAAATTCTATGCATGGGTTTGGATTTATGACAGGCCCGTCACCCTGGGCCTGTCA
TAGTACCCCATGCCAGAGCAAACTGTGTCCCCGAACCATTGCCTGGCCTCTGTGCCCGTAGGCTGCTGGC
ACTGAAGTGGGTTGCACAGTGGAAAAGAAGAAAGCTCTACCTGGCAGAAATTTTTAAAGGTTAAAATAAA
TAATTTTAAGAAAGCTGGTTCACAAGGTGCCACATTTGATGAAAGCAAAATACAGTGGCTTTTATTGTTA
CTAGAGTGATGTTCTTGCTTGTTTTTCTTTTTTGGTGAAGTTAGCCCCAAATTATTCTCATAGCTAAGCA
AATACGAGAGTGACTGTAAGGACAGTTGGCATTCCCGGAATTGCTAAACTTGGTAGGCAACGCTGGTTTA
AGAATACTGAGTTCTAGCCGGGCGTGGTGGCTCACGCCTGTAATCCCAACACTTTGGGAGGCTGAGGCAG
GCGGATCACCTGAGGTCGGGAGTTGGAGACCAGCCTGACTAACATGGAGAAACGCCATCTCCACTAAAAA
TATAAAATTAGCCAGGCCCCGGGTGTGGTGGCACATGCCGGTAATCCCAGCTACTCGGGAGACTGAGGCA
GGAGAATCGCTTGAACCCAGGAGGCGGAGGTTGAGGTGAGCCGAGATCATGCCATTGCACTCCAGCCTGG
GCAACAAGAGTAAAACTCTGTCTCAAAAAAAAAAAAAAAAAATACTGAATTCTGATCAGGTAACAGCAAC
TGTAATACAATGTGATAAGTTGACTTGAAGATTACAGTTTTTAAGAAGTATATACCCAGCTAATACATGA
AAATTAACTCGTAAAATCTCAAATGCTCCAGACATTTCCATGATGCCTGTTGGTCAGTAAAAATCATTCT
AAGACTTAGTGGAAGTAGGAAATGTTTGTATGGCTGTGTATAAAGGCTATAATGTAATCCCAGCACTTTG
GAAGACCGAGGCGGGTGGATCACCTGGGGTCAGGAGTTTGAGACCCACCTGGACAACGTGGTGAAATCCT
GTCTCTACTAAAAACACAAAAATTAGCCGGGCATGGTGGCAGGCGCCTGTAATCCCAGCTGCTGGGGAGG
CTGAGGCAGGAGAATCGCTTGAACCCGGGAGGCAGAGGTTGCAGTGAGCCAAGATTGCACCGCTGCACTC
CAGCCTGGGTGACAGCGTGAGACTCTGTCTCAAAAAAAATAAAAAAGTCTATAATGCTATTTTAAGTTTC
TAAGGAACTGAAACTGCTCTGAAATAAATCAGACCATTATAAGACTTTTTTCCATATCAGTGAGCTAAGT
GCAGATAAGCTTCTGAAACTTGCATGCTAGATTTTTTTGGTACAAATATTTGAAATGCTTAGTGTGCTGC
CTTGGAAAAACCTGGTATTTTTTGTTGTGTCCTTATACTGCCAAGGTTTATGGAATCATGTACCTTATGC
CTAGTAATAATTAGGATGACCAGGCCAGTGAGTGGTTCATATCCGGGGCATGATTAGCTCTGCGTGTGCT
CAGCCAGTGCCCCATCTTCAACTCGATGTGTTCCTAAGGTAGACAGCAAATTCCCTATTTTATTTCTCAG
ATTGTCACTGCTGTTCCAAGGGCACACGCAGAGGGATTTGGAATTCCTGGAGAGTTGCCTTTGTGAGAAG
CTGGAAATATTTCTTTCAATTCCATCTCTTAGTTTTCCATGTAAGTATTCAGTTTACATTTATGTTGCAG
GTTAATCTTAAGAATTGTATTGCTAAGGCTTCTAAGTGAATTTCTCCACTCTATTTGCATTTTGTTGCAT
TTCAGAGGAACATCAAGAAATCATGAACAACTTTGGTAATGAAGAGTTTGACTGCCACTTCCTCGATGAA
GGTTTTACTGCCAAGGACATTCTGGACCAGAAAATTAATGAAGTTTCTTCTTCTGTAAGTATATGAGGCC
CATGCTGGCAGTGCAGCTGAGAGTGCCAGGCAAGTGGAAAACTTTGGCAAGGTCTAAGGAAGAGCAATGA
GGCTTACATGTCTTGTTATGGAATGTAGAAATTAATTCACTGGTGGTAAATTAATAGTGATAATGGTGAT
ACTCATATCAGTGGCTAGACTCAAAAGAGCAGGATTCATTGTGACTGATGGGAATGAAGGTCGCTGGCTA
TTGGTGTGGTGTGTGGTGAGGCTGCTAGTGAGTCACCTGTGACCACTCTTGTTTCAGGATGATAAGGATG
CCTTCTATGTGGCAGACCTGGGAGACATTCTAAAGAAACATCTGAGGTGGTTAAAAGCTCTCCCTCGTGT
CACCCCCTTTTATGCAGTCAAATGTAATGATAGCAAAGCCATCGTGAAGACCCTTGCTGCTACCGGGACA
GGATTTGACTGTGCTAGCAAGGTAAGCGATAGCAGCAGGCCTCAAAAGCGTTGTATAAAATGGGCCTGGT
ATTCCCCACGAGGCAGATACAAGTTGTGTTTTTTGGGCAATAAATGCTCACTAAAGGCAAATGGGGCGGG
GGGGTACATGACAACTTCCCATGCTTTTCTGTTTATTCCACGTGTTAAGCCACATATGGATAGCATGACA
CCACTCTTCTTTTTCAGACTGAAATACAGTTGGTGCAGAGTCTGGGGGTGCCTCCAGAGAGGATTATCTA
TGCAAATCCTTGTAAACAAGTATCTCAAATTAAGTATGCTGCTAATAATGGAGTCCAGATGATGACTTTT
GATAGTGAAGTTGAGTTGATGAAAGTTGCCAGAGCACATCCCAAAGCAAAGTGAGTTATTCCCCCATCTG
AGGGCAAGATCGGGAGCATAAGATATGTGGATTCTTATCAAACAAACTTAAATTTCTGATTATTATATTT
CTATACTTTAGTAGAAAGTAGTTGAAACCCCCATTGAGTCATGAAGCCTGGGACTCAAACTACAGAATAT
ATCAGCGACAGTATTTAGAACAGGATTGTTTTTATTTTAATTGTGGCTATAAGTGAACATCTATCATGAG
ACATTTGCTGCACTTTCCTTGCTTGTAGGTTGGTTTTGCGGATTGCCACTGATGATTCCAAAGCAGTCTG
TCGTCTCAGTGTGAAATTCGGTGCCACGCTCAGAACCAGCAGGCTCCTTTTGGAACGGGCGAAAGAGCTA
AATATCGATGTTGTTGGTGTCAGGTGAGATTTTGGTGGGATAGCTAGAGGTCAAGACATTGAACAGTTTG
AGTTTTACAGGCTTTCTCCTAGTGTTTGCTATTATTTTAAGAAATACTAAGACACAGTGTCTCGTCTCTT
TATTTTACCCCAGCTTCCATGTAGGAAGCGGCTGTACCGATCCTGAGACCTTCGTGCAGGCAATCTCTGA
TGCCCGCTGTGTTTTTGACATGGGGGTGAGTATACGTGACCCTGTTAGGGAAGGGCGGGACACAACTGAC
AATAACTAGTCTTAATTCTAGAGTTAACTTTTTATGGCAGTTGGTTCTGTATTACATGGGTTTCAGCCTA
TCTGCTGCATACATTTTTGTTATTAGCTGTGGATCTGGCTGACTTATTTTCTTGATTCTAGGCTGAGGTT
GGTTTCAGCATGTATCTGCTTGATATTGGCGGTGGCTTTCCTGGATCTGAGGATGTGAAACTTAAATTTG
AAGAGGTAATTTAGAACAAAACTGTAATACTCAGTAGCCGTTCTAATAAATTCCTTTTTGGAATATTTCA
AAATTTAAGTGTCTTAACTAATACCACAATGGGCTGAAGTGTCTTGGTGTGATATTTTGAGTGATTTCTT
TGTGCTGTCTGACATTACACTTGATACCATTTGGTTTTCTAAAGTGTGAATCAGCTTTCCCAGAAGTCTT
GGATAATTGGTTACATTGGAAATCATGGCTCACACCTGTAATCCAGCACTTGGGGAGGCCAAGGTGGTAG
GATCACTTGAGCCCAGGAGTTTGAGACCAGCCTGGGCAACACAGTGAGACCCCATCTCTACAAAAAAAAT
TTTAAAATTAGCCTGGTGTGGTGGCGGGCACCTGTAATCCCAGCTACTTGGAAGGCTGAGGTGGGAGGAT
CACTTGAGCCCAGGAGGTTGAGGCTGCAGTGAGCCATGATCATGCCACTGCACTCAGCCTGGGCTACAGA
GTGAGACCCTGTCTCAAAAAAAAAAAAGAAAAAGCATGTTGCTGTGGGCTTCCTAGAGAATATGCTGACT
GTAGCACATCATCACCCCAAATGTGCTTTGCTAGACCTATGCTTCCTCTCCTTAAAATACTTGAAATGTT
TAGTCACTTAGGAAGTTAAGCCATTATATTGGTGCTTGAATTTATAAAATATATCCACATGGTTTGTTAA
AATCATGACGTAGGCAGAATAGGATTTTTATCCTGTTGGCATGTATTTGTTAAAATGTTTTGACATCTTG
ATGCCTTCCTAGGTAGTAGTTAGTTGCGTACTGTTCTTTGATAAAAATCATACCCATAACATCCTAAAGG
AGATAGGGTGCCTGGAGGGGAATGAAAACGAGCCACCTGGGATATGTAGCCTGGTTTTCAGGGAGATGTT
GATGTTTTTTTGCTTTTGTTACTTTAATGATAAACCTGTCTGTTGATGCCTGGTCTCATGATGTCATGTC
ACAAGGCCCTGTGATGTTACTCCCCCATGTGAATTTCCCACAATGAAGGCTGCTCTTTCTTTTCTGTTTC
ACTCTCTTAGATCACCGGCGTAATCAACCCAGCGTTGGACAAATACTTTCCGTCAGACTCTGGAGTGAGA
ATCATAGCTGAGCCCGGCAGATACTATGTTGCATCAGCTTTCACGCTTGCAGTTAATATCATTGCCAAGA
AAATTGTATTAAAGGAACAGACGGGCTCTGATGGTATGTATAAAGGACGAATCACTTCATGTATAACTGA
AAGCTGATGCAAAAAGTCATTAAGATTGTTGATCTGCCTTTCTAGACGAAGATGAGTCGAGTGAGCAGAC
CTTTATGTATTATGTGAATGATGGCGTCTATGGATCATTTAATTGCATACTCTATGACCACGCACATGTA
AAGCCCCTTCTGCAAAAGGTAATTTCTGAGCATACTGTATAAAACAATTAAGAGGACTGGTCACAACACG
TGTAATTAAGTAGTACTTCCTCTCTCCGTCTCTTTATATAGAGACCTAAACCAGATGAGAAGTATTATTC
ATCCAGCATATGGGGACCAACATGTGATGGCCTCGATCGGATTGTTGAGCGCTGTGACCTGCCTGAAATG
CATGTGGGTGATTGGATGCTCTTTGAAAACATGGGCGCTTACACTGTTGCTGCTGCCTCTACGTTCAATG
GCTTCCAGAGGCCGACGATCTACTATGTGATGTCAGGGCCTGCGTGGTAAGTAAGCCATGCATGTTGATG
GTGCTGCCAAGAATAGGCACCTTCTTGGATGTGTGCTTCTTGTCTAGACGAATAAGAAATTGTCTTGCCT
AAGATTAAATATATATGGATATTTTTCCTAAGAAAAGTTTTAGAAAAGACTGATGAGTGTATTTCTATGT
AATTGGAATATATTTAAGTTCATGCCATGTGTCTTGTGGTTTCCTTATTACCAAAACGGTGACTGAAGAA
ACGCTTGCTTTAGAAATACATTGAATTGGCCAGGTGTGCTGGCTCACACCTGAAATCACAACACATTGGG
AGGCCAAGGCAGAAGGATCACTTGAGCCCAGGAGTTCGAGCCTGGGCAACATAGTGAGACCCTGTCTCTA
CAAAAAATTAAAAAATTAGTTGGCCATGGTAGTGGGCGCCTGTAGTCCCAGCTGCTTGGCTAAGGTGAGA
GGTTTGCTTGAGCCTGGGAGGTTGAGGCTGCGGTGAGCTATGATAGCACCATTGTATTCCAGCCTGAGTA
ACAGAGAAAGACCCTGTCTCAGAAAAAAAAAAAATACATTGAATTGTTTCCTGATGGGAAGTAAATACTC
TCATGCCCAGTTAGGAGTGAGTCAGGGTTTTTAATATGCCACTTTTTCTTTCTCAGGCAACTCATGCAGC
AATTCCAGAACCCCGACTTCCCACCCGAAGTAGAGGAACAGGATGCCAGCACCCTGCCTGTGTCTTGTGC
CTGGGAGAGTGGGATGAAACGCCACAGAGCAGCCTGTGCTTCGGCTAGTATTAATGTGTAGATAGCACTC
TGGTAGCTGTTAACTGCAAGTTTAGCTTGAATTAAGGGATTTGGGGGGACCATGTAACTTAATTACTGCT
AGTTTTGAAATGTCTTTGTAAGAGTAGGGTCGCCATGATGCAGCCATATGGAAGACTAGGATATGGGTCA
CACTTATCTGTGTTCCTATGGAAACTATTTGAATATTTGTTTTATATGGATTTTTATTCACTCTTCAGAC
ACGCTACTCAAGAGTGCCCCTCAGCTGCTGAACAAGCATTTGTAGCTTGTACAATGGCAGAATGGGCCAA
AAGCTTAGTGTTGTGACCTGTTTTTAAAATAAAGTATCTTGAAATAATTAGGCATTGGGACGTT
DNA sequence encoding ArgG
>NC_000913.3:3318637-3319980 Escherichia coli str. K-12 substr. MG1655, complete
genome
Sequence ID NO: 32
ATGACGACGATTCTCAAGCATCTCCCGGTAGGTCAACGTATTGGTATCGCTTTTTCTGGCGGTCTGGACA
CCAGTGCCGCACTGCTGTGGATGCGACAAAAGGGAGCGGTTCCTTATGCATATACTGCAAACCTGGGCCA
GCCAGACGAAGAGGATTATGATGCGATCCCTCGTCGTGCCATGGAATACGGCGCGGAGAACGCACGTCTG
ATCGACTGCCGCAAACAACTGGTGGCCGAAGGTATTGCCGCTATTCAGTGTGGCGCATTTCATAACACCA
CCGGCGGCCTGACCTATTTCAACACGACGCCGCTGGGCCGCGCCGTGACTGGTACCATGCTGGTTGCTGC
GATGAAAGAAGATGGCGTGAATATCTGGGGTGACGGTAGCACCTACAAAGGAAACGATATCGAACGTTTC
TATCGTTATGGTCTGCTGACCAATGCTGAACTGCAGATTTACAAACCGTGGCTTGATACTGACTTTATTG
ATGAACTGGGCGGCCGTCATGAGATGTCTGAATTTATGATTGCCTGCGGTTTCGACTACAAAATGTCTGT
CGAAAAAGCCTACTCCACAGACTCCAACATGCTTGGTGCAACGCATGAAGCGAAGGATCTGGAATACCTC
AACTCCAGCGTCAAAATCGTCAACCCGATTATGGGCGTGAAATTCTGGGATGAGAGCGTGAAGATCCCGG
CAGAAGAAGTCACAGTACGCTTTGAACAAGGTCATCCGGTGGCGCTGAACGGTAAAACCTTTAGCGACGA
CGTAGAAATGATGCTGGAAGCTAACCGCATCGGCGGTCGTCACGGCCTGGGCATGAGCGACCAGATTGAA
AACCGTATCATCGAAGCGAAAAGCCGTGGTATTTACGAAGCTCCGGGGATGGCACTGCTGCACATTGCGT
ATGAACGCCTGTTGACCGGTATTCACAACGAAGACACCATTGAGCAGTATCACGCGCATGGTCGTCAGTT
GGGCCGTCTGCTGTACCAGGGGCGTTGGTTTGACTCCCAGGCGCTGATGCTGCGTGACTCTCTGCAACGC
TGGGTTGCCAGCCAGATCACTGGTGAAGTTACCCTGGAGCTGCGCCGTGGGAACGATTATTCAATCCTGA
ATACCGTCTCAGAGAACCTGACCTACAAGCCAGAGCGTCTGACGATGGAAAAAGGCGACTCGGTGTTCTC
GCCAGATGATCGTATTGGTCAATTGACCATGCGTAACCTGGATATCACTGATACCCGCGAGAAACTTTTC
GGTTATGCCAAAACTGGCCTGCTTTCCTCCTCTGCCGCTTCAGGCGTGCCGCAGGTGGAGAATCTGGAAA
ACAAAGGCCAGTAA
DNA sequence encoding ArgH
>NC_000913.3:4156850-4158223 Escherichia coli str. K-12 substr. MG1655, complete
genome
Sequence ID NO: 33
ATGGCACTTTGGGGCGGGCGTTTTACCCAGGCAGCAGATCAACGGTTCAAACAATTCAACGACT
CACTGCGCTTTGATTACCGTCTGGCGGAGCAGGATATTGTTGGCTCTGTGGCCTGGTCCAAAGC
CCTGGTCACGGTAGGCGTGTTAACCGCAGAAGAGCAGGCGCAACTGGAAGAGGCGCTGAACGT
GTTGCTGGAAGATGTTCGCGCCAGGCCACAACAAATCCTTGAAAGCGACGCCGAAGATATCCAT
AGCTGGGTGGAAGGCAAACTGATCGACAAAGTGGGCCAGTTAGGCAAAAAGCTGCATACCGGG
CGTAGCCGTAATGATCAGGTAGCGACTGACCTGAAACTGTGGTGCAAAGATACCGTTAGCGAGT
TACTGACGGCTAACCGGCAGCTGCAATCGGCGCTGGTGGAAACCGCACAAAACAATCAGGACG
CGGTAATGCCAGGTTACACTCACCTGCAACGCGCCCAGCCGGTGACGTTCGCGCACTGGTGCC
TGGCCTATGTTGAGATGCTGGCGCGTGATGAAAGCCGTTTGCAGGATGCGCTTAAGCGTCTGGA
TGTCAGCCCGCTAGGCTGTGGCGCGCTGGCGGGAACGGCCTATGAAATCGACCGTGAACAGTT
AGCAGGCTGGCTGGGCTTTGCTTCGGCGACCCGTAACAGTCTCGACAGCGTTTCTGACCGTGAC
CATGTGTTGGAACTGCTTTCTGCTGCCGCTATCGGCATGGTGCATCTGTCGCGTTTTGCTGAAGA
TCTGATTTTCTTTAACACCGGCGAAGCGGGGTTTGTGGAGCTTTCTGACCGCGTGACTTCCGGTT
CATCATTAATGCCGCAGAAGAAAAACCCGGATGCGCTGGAGCTGATTCGCGGTAAATGCGGCCG
GGTGCAGGGGGCGTTAACCGGCATGATGATGACGCTGAAAGGTTTGCCGCTGGCTTACAACAAA
GATATGCAGGAAGACAAAGAAGGTCTGTTCGACGCGCTCGATACCTGGCTGGACTGCCTGCATA
TGGCGGCGCTGGTGCTGGACGGCATTCAGGTGAAACGTCCACGTTGCCAGGAAGCGGCTCAGC
AGGGTTACGCCAACGCCACCGAACTGGCGGATTATCTGGTGGCGAAAGGCGTACCGTTCCGCG
AGGCGCACCATATTGTTGGTGAAGCGGTGGTGGAAGCCATTCGTCAGGGCAAACCGCTGGAAG
ATCTGCCGCTCAGTGAGTTGCAGAAATTCAGTCAGGTGATTGACGAAGATGTCTATCCGATTCTG
TCGCTGCAATCGTGCCTCGACAAGCGTGCGGCAAAAGGCGGCGTCTCACCGCAGCAGGTGGCG
CAGGCGATTGCTTTTGCGCAGGCTCGGTTAGGGTAA
DNA sequence encoding ArgF
>NC_000913.3:c290305-289301 Escherichia coli str. K-12 substr. MG1655, complete
genome
Sequence ID NO: 34
ATGTCCGATTTATACAAAAAACACTTTCTGAAACTGCTCGACTTTACCCCTGCACAGTTCACTTCT
CTGCTGACCCTTGCCGCACAGCTCAAAGCCGATAAAAAAAATGGCAAGGAAGTACAGAAGCTTA
CCGGTAAAAACATCGCGCTCATCTTCGAAAAAGACTCGACTCGTACCCGTTGCTCTTTCGAAGTT
GCCGCATTTGACCAGGGCGCGCGCGTTACCTATTTAGGGCCGAGCGGCAGCCAGATTGGGCAT
AAAGAGTCAATTAAGGACACCGCGCGGGTTCTCGGGCGGATGTATGACGGCATTCAGTATCGCG
GTCACGGCCAGGAAGTGGTCGAAACGCT
GGCGCAGTATGCGGGCGTGCCGGTGTGGAACGGGCTGACCAACGAGTTCCACCCGACCCAGC
TGCTGGCGGACCTGATGACCATGCAGGAGCACCTGCCGGGCAAGGCGTTTAACGAGATGACGC
TGGTCTACGCGGGCGATGCGCGCAACAACATGGGCAACTCGATGCTGGAAGCGGCGGCGCTGA
CCGGGCTGGATCTGCGCCTGTTGGCCCCGAAAGCCTGCTGGCCGGAAGAGAGCCTGGTGGCG
GAGTGCAGCGCGCTGGCGGAGAAGCACGGCGGGAAAATTACTCTGACGGAAGACGTGGCGGC
AGGCGTTAAGGGCGCGGACTTTATCTATACCGACGTGTGGGTGTCGATGGGCGAGGCCAAAGA
GAAGTGGGCAGAGCGGATTGCGCTGCTGCGCGGGTATCAGGTGAACGCGCAGATGATGGCGCT
GACCGACAACCCGAACGTGAAGTTCCTGCACTGTCTGCCGGCGTTCCATGACGACCAGACTACG
CTCGGCAAGCAGATGGCGAAGGAGTTCGATCTGCACGGCGGGATGGAGGTGACGGACGAGGT
GTTTGAGTCGGCGGCGAGCATCGTGTTCGACCAGGCGGAAAACCGGATGCATACGATTAAGGC
GGTGATGATGGCAACGCTTGGGGAGTGA
DNA sequence encoding Trp5
>NC_001139.9:c448535-446412 Saccharomyces cerevisiae S288c chromosome VII, complete
sequence
Sequence ID NO: 35
ATGTCAGAACAACTCAGACAAACATTTGCTAACGCTAAAAAAGAAAACAGGAACGCCTTGGTCAC
ATTTATGACCGCAGGTTACCCAACAGTCAAAGACACTGTCCCTATTCTCAAGGGTTTCCAGGATG
GTGGTGTAGATATCATCGAATTGGGTATGCCCTTCTCTGATCCAATTGCAGATGGTCCTACAATT
CAATTATCTAATACTGTGGCTTTGCAAAACGGTGTTACCTTGCCTCAAACTCTAGAAATGGTCTCC
CAAGCTAGAAATGAAGGTGTTACCGTACCCATAATCCTAATGGGTTACTATAACCCTATTCTAAAC
TACGGTGAAGAAAGATTTATTCAGGACGCTGCCAAGGCTGGTGCTAATGGTTTTATCATCGTCGA
TTTGCCACCAGAGGAGGCGTTGAAGGTCAGAAACTACATCAATGATAATGGTTTGAGCCTGATCC
CACTAGTGGCTCCTTCTACCACCGACGAAAGATTGGAATTACTATCGCATATTGCCGATTCGTTT
GTCTACGTTGTGTCTAGAATGGGTACTACTGGTGTTCAAAGTTCTGTGGCCAGTGATTTGGATGA
ACTCATCTCTAGAGTCAGAAAGTACACCAAGGATACTCCTTTGGCCGTTGGGTTTGGTGTCTCTA
CCAGAGAACATTTCCAATCAGTTGGTAGTGTTGCTGACGGTGTAGTGATTGGTTCCAAAATCGTC
ACATTATGTGGAGATGCTCCAGAGGGCAAAAGGTACGACGTTGCTAAGGAATATGTACAGGGAA
TTCTAAATGGTGCTAAGCATAAGGTTCTGTCCAAGGACGAATTCTTTGCCTTTCAAAAAGAGTCCT
TGAAGTCCGCAAACGTTAAGAAGGAAATACTGGACGAATTTGATGAAAATCACAAGCACCCAATT
AGATTTGGGGACTTTGGTGGTCAGTATGTCCCAGAAGCTCTTCATGCATGTCTAAGAGAGTTGGA
AAAGGGTTTTGATGAAGCTGTCGCCGATCCCACATTCTGGGAAGACTTCAAATCCTTGTATTCTT
ATATTGGCCGTCCTTCTTCACTACACAAAGCTGAGAGATTAACTGAGCATTGTCAAGGTGCTCAA
ATCTGGTTGAAGAGAGAAGATCTTAACCACACGGGATCTCACAAGATCAACAATGCTTTAGCACA
AGTTCTTCTAGCTAAAAGATTAGGCAAGAAGAACGTTATTGCTGAAACCGGTGCTGGTCAACACG
GTGTTGCCACTGCCACTGCATGTGCTAAATTTGGCTTAACCTGTACTGTGTTCATGGGTGCAGAA
GATGTTCGTCGCCAAGCTTTAAACGTCTTCAGAATGAGAATTCTCGGTGCTAAAGTAATTGCTGTT
ACTAATGGTACAAAGACTCTAAGAGACGCTACTTCAGAGGCATTCAGATTTTGGGTTACTAACTT
GAAAACTACTTACTACGTCGTCGGTTCTGCCATTGGTCCTCACCCATATCCAACTTTGGTTAGAA
CTTTCCAAAGTGTCATTGGTAAAGAAACCAAGGAACAGTTTGCTGCCATGAACAATGGTAAATTA
CCTGACGCAGTTGTTGCATGTGTTGGGGGTGGTTCCAACTCTACAGGTATGTTTTCACCATTCGA
GCATGATACTTCCGTTAAGTTATTGGGTGTGGAAGCCGGTGGTGATGGTGTAGATACAAAGTTCC
ACTCTGCTACTCTAACTGCCGGTAGACCTGGTGTCTTCCATGGTGTCAAGACTTATGTCTTGCAA
GATAGTGATGGTCAAGTCCATGATACTCATTCTGTTTCTGCTGGGTTAGACTACCCAGGTGTCGG
TCCAGAATTGGCATATTGGAAATCTACTGGCCGTGCTCAATTCATTGCAGCTACTGACGCTCAGG
CTCTGCTTGGCTTTAAATTATTATCTCAATTAGAAGGTATTATTCCCGCTTTGGAATCTTCTCATGC
TGTTTATGGCGCTTGCGAATTGGCTAAGACGATGAAGCCTGATCAACATTTGGTTATCAATATTTC
TGGTAGAGGTGATAAAGATGTCCAAAGTGTCGCTGAAGTCTTGCCGAAATTAGGTCCAAAGATAG
GTTGGGATTTGAGATTCGAAGAAGACCCATCTGCCTAA
DNA sequence encoding IDO
>NC_001139.9:c448535-446412 Saccharomyces cerevisiae S288c chromosome VII,
complete sequence
>NC_001139.9:c448535-446412 Saccharomyces cerevisiae S288c chromosome VII,
complete sequence
Sequence ID NO: 36
ATGTCAGAACAACTCAGACAAACATTTGCTAACGCTAAAAAAGAAAACAGGAACGCCTTGGTCAC
ATTTATGACCGCAGGTTACCCAACAGTCAAAGACACTGTCCCTATTCTCAAGGGTTTCCAGGATG
GTGGTGTAGATATCATCGAATTGGGTATGCCCTTCTCTGATCCAATTGCAGATGGTCCTACAATT
CAATTATCTAATACTGTGGCTTTGCAAAACGGTGTTACCTTGCCTCAAACTCTAGAAATGGTCTCC
CAAGCTAGAAATGAAGGTGTTACCGTACCCATAATCCTAATGGGTTACTATAACCCTATTCTAAAC
TACGGTGAAGAAAGATTTATTCAGGACGCTGCCAAGGCTGGTGCTAATGGTTTTATCATCGTCGA
TTTGCCACCAGAGGAGGCGTTGAAGGTCAGAAACTACATCAATGATAATGGTTTGAGCCTGATCC
CACTAGTGGCTCCTTCTACCACCGACGAAAGATTGGAATTACTATCGCATATTGCCGATTCGTTT
GTCTACGTTGTGTCTAGAATGGGTACTACTGGTGTTCAAAGTTCTGTGGCCAGTGATTTGGATGA
ACTCATCTCTAGAGTCAGAAAGTACACCAAGGATACTCCTTTGGCCGTTGGGTTTGGTGTCTCTA
CCAGAGAACATTTCCAATCAGTTGGTAGTGTTGCTGACGGTGTAGTGATTGGTTCCAAAATCGTC
ACATTATGTGGAGATGCTCCAGAGGGCAAAAGGTACGACGTTGCTAAGGAATATGTACAGGGAA
TTCTAAATGGTGCTAAGCATAAGGTTCTGTCCAAGGACGAATTCTTTGCCTTTCAAAAAGAGTCCT
TGAAGTCCGCAAACGTTAAGAAGGAAATACTGGACGAATTTGATGAAAATCACAAGCACCCAA
TTAGATTTGGGGACTTTGGTGGTCAGTATGTCCCAGAAGCTCTTCATGCATGTCTAAGAGAGTTG
GAAAAGGGTTTTGATGAAGCTGTCGCCGATCCCACATTCTGGGAAGACTTCAAATCCTTGTATTC
TTATATTGGCCGTCCTTCTTCACTACACAAAGCTGAGAGATTAACTGAGCATTGTCAAGGTGCTC
AAATCTGGTTGAAGAGAGAAGATCTTAACCACACGGGATCTCACAAGATCAACAATGCTTTAGCA
CAAGTTCTTCTAGCTAAAAGATTAGGCAAGAAGAACGTTATTGCTGAAACCGGTGCTGGTCAACA
CGGTGTTGCCACTGCCACTGCATGTGCTAAATTTGGCTTAACCTGTACTGTGTTCATGGGTGCAG
AAGATGTTCGTCGCCAAGCTTTAAACGTCTTCAGAATGAGAATTCTCGGTGCTAAAGTAATTGCT
GTTACTAATGGTACAAAGACTCTAAGAGACGCTACTTCAGAGGCATTCAGATTTTGGGTTACTAA
CTTGAAAACTACTTACTACGTCGTCGGTTCTGCCATTGGTCCTCACCCATATCCAACTTTGGTTAG
AACTTTCCAAAGTGTCATTGGTAAAGAAACCAAGGAACAGTTTGCTGCCATGAACAATGGTAAATT
ACCTGACGCAGTTGTTGCATGTGTTGGGGGTGGTTCCAACTCTACAGGTATGTTTTCACCATTCG
AGCATGATACTTCCGTTAAGTTATTGGGTGTGGAAGCCGGTGGTGATGGTGTAGATACAAAGTTC
CACTCTGCTACTCTAACTGCCGGTAGACCTGGTGTCTTCCATGGTGTCAAGACTTATGTCTTGCA
AGATAGTGATGGTCAAGTCCATGATACTCATTCTGTTTCTGCTGGGTTAGACTACCCAGGTGTCG
GTCCAGAATTGGCATATTGGAAATCTACTGGCCGTGCTCAATTCATTGCAGCTACTGACGCTCAG
GCTCTGCTTGGCTTTAAATTATTATCTCAATTAGAAGGTATTATTCCCGCTTTGGAATCTTCTCATG
CTGTTTATGGCGCTTGCGAATTGGCTAAGACGATGAAGCCTGATCAACATTTGGTTATCAATATTT
CTGGTAGAGGTGATAAAGATGTCCAAAGTGTCGCTGAAGTCTTGCCGAAATTAGGTCCAAAGATA
GGTTGGGATTTGAGATTCGAAGAAGACCCATCTGCCTAA
DNA sequence encoding exemplary CAR GD2 ASS1 + OTC without MP71
vector
Sequence ID NO: 37
ATGCCTCGCGGCTGGACAGCCCTGTGCCTGCTGTCTCTGCTGCCATCCGGCTTCATGAGCCTG
GATAATAACGGCACAGCCACCCCAGAGCTGCCTACACAGGGCACCTTCAGCAATGTGTCCACAA
ACGTGAGCTATCAGGAGACCACAACCCCTTCTACCCTGGGATCCACAAGCCTGCACCCCGTGTC
TCAGCACGGCAACGAAGCCACCACCAACATCACCGAGACCACAGTGAAGTTTACCTCCACCTCT
GTGATTACCTCTGTGTACGGAAATACAAACTCCAGCGTGCAGTCTCAGACATCTGTGATCTCCAC
AGTGTTTACAACACCTGCCAATGTGTCCACCCCAGAGACAACCCTGAAGCCCAGCCTGTCTCCT
GGAAATGTGTCCGATCTGTCTACCACCTCCACCAGCCTGGCCACCTCTCCCACCAAGCCCTATA
CCTCCTCTTCTCCCATCCTGAGCGATATCAAAGCCGAGATCAAATGCAGCGGGATTCGGGAAGT
GAAACTGACACAGGGCATCTGCCTGGAACAGAATAAGACATCCAGCTGCGCCGAGTTTAAGAAA
GATAGAGGAGAGGGACTGGCCAGGGTGCTGTGTGGCGAAGAGCAGGCCGACGCCGATGCCGG
CGCCCAGGTGTGTTCCCTGCTGCTGGCCCAGTCTGAGGTGCGCCCCCAGTGCCTGCTGCTGGT
GCTGGCCAATCGGACAGAAATTAGCAGCAAGCTGCAGCTGATGAAAAAACACCAGAGCGATCTG
AAAAAGCTGGGCATCCTGGACTTTACCGAGCAGGACGTGGCCTCTCACCAGAGCTACAGCCAGA
AAACACTGATCGCCCTGGTGACCAGCGGAGCCCTGCTGGCCGTGCTGGGCATCACCGGATATT
TCCTGATGAATAGGCGCAGCTGGAGCCCCACCGGCGAACGGCTGGAGCTGGAGCCTGTCGACC
GAGTGAAGCAGACCCTGAACTTTGATCTGCTGAAGCTGGCCGGCGACGTGGAGTCCAACCCCG
GGCCAGGGAATATGGCCTTACCAGTGACCGCCTTGCTCCTGCCGCTGGCCTTGCTGCTCCACG
CCGCCAGGCCGGATATTCTGCTCACACAGACCCCACTCTCCCTGCCCGTGTCACTCGGGGATCA
GGCTAGCATTTCTTGCCGCTCATCTCAGTCTCTGGTCCACCGGAATGGGAACACATACCTCCATT
GGTACCTCCAGAAACCTGGACAGAGCCCTAAACTGCTCATCCACAAAGTCTCAAATCGGTTCTCC
GGCGTGCCCGATCGCTTTAGCGGATCCGGATCTGGGACCGACTTCACACTGAAAATCTCACGAG
TGGAGGCTGAGGATCTCGGCGTCTACTTCTGTAGTCAGAGTACCCACGTCCCACCCCTCACCTT
TGGCGCTGGAACAAAACTGGAGCTGAAACGAGCCGATGCTGCTCCTACCGTGTCCATCTTTCCT
GGCTCCGGGGGAGGCGGGAGCGGAGGCGAAGTGAAACTCCAGCAGTCTGGCCCTTCTCTCGT
GGAACCTGGCGCTTCTGTGATGATCTCCTGTAAGGCCTCTGGATCTTCCTTTACCGGCTACAACA
TGAACTGGGTCCGGCAGAACATTGGCAAATCCCTGGAATGGATTGGCGCCATCGATCCTTACTA
CGGCGGCACATCATACAATCAGAAATTCAAGGGGCGAGCAACACTCACTGTCGACAAATCTTCAT
CCACCGCCTACATGCACCTGAAATCTCTCACATCCGAGGATAGTGCTGTCTACTACTGTGTCTCT
GGCATGGAATACTGGGGACAGGGAACTTCTGTCACCGTGTCTAGTGCCAAAACCACACCTCCCT
CCGTGTACGGACGAGTCACTGTCTCATCTGCTGAACCAAAATCCTGTGACAAAACACACACATGC
CCACCTTGTCCTGCCCCTGAACTGCTCGGCGGACCTTCCGTCTTTCTGTTTCCCCCCAAACCCAA
GGATACACTCATGATTTCTAGGACCCCCGAAGTCACTTGTGTCGTGGTCGATGTGTCTCACGAG
GATCCTGAAGTGAAATTCAACTGGTACGTGGACGGAGTCGAGGTCCACAATGCCAAAACAAAAC
CCCGGGAGGAACAGTACAATAGCACCTACCGAGTCGTGTCCGTGCTCACCGTCCTCCATCAGGA
TTGGCTGAACGGCAAAGAGTACAAGTGTAAAGTGAGTAACAAGGCTCTCCCCGCTCCTATTGAAA
AAACCATCTCAAAAGCAAAAGGCCAGCCTAGGGAGCCTCAGGTCTACACACTGCCACCCTCACG
GGACGAACTCACCAAAAATCAGGTGTCCCTCACTTGCCTGGTGAAAGGCTTCTACCCTTCCGATA
TCGCTGTGGAATGGGAGTCAAATGGGCAGCCCGAAAACAACTACAAAACAACCCCCCCTGTGCT
CGATTCCGATGGCTCTTTTTTCCTGTACTCCAAACTCACCGTGGACAAATCACGCTGGCAGCAGG
GGAATGTCTTTTCTTGCTCCGTGATGCACGAGGCCCTCCACAATCATTACACCCAGAAATCCCTC
TCACTCTCACCCGGCAAAAAGGACCCTAAAACCACGACGCCAGCGCCGCGACCACCAACACCG
GCGCCCACCATCGCGTCGCAGCCCCTGTCCCTGCGCCCAGAGGCGTGCCGGCCAGCGGCGGG
GGGCGCAGTGCACACGAGGGGGCTGGACTTCGCCTGTGATATCTACATCTGGGCGCCCTTGGC
CGGGACTTGTGGGGTCCTTCTCCTGTCACTGGTTATCACCCTTTACTGCAAACGGGGCAGAAAG
AAACTCCTGTATATATTCAAACAACCATTTATGAGACCAGTACAAACTACTCAAGAGGAAGATGGC
TGTAGCTGCCGATTTCCAGAAGAAGAAGAAGGAGGATGTGAACTGAGAGTGAAGTTCAGCAGGA
GCGCAGACGCCCCCGCGTACAAGCAGGGCCAGAACCAGCTCTATAACGAGCTCAATCTAGGAC
GAAGAGAGGAGTACGATGTTTTGGACAAGAGACGTGGCCGGGACCCTGAGATGGGGGGAAAGC
CGAGAAGGAAGAACCCTCAGGAAGGCCTGTACAATGAACTGCAGAAAGATAAGATGGCGGAGG
CCTACAGTGAGATTGGGATGAAAGGCGAGCGCCGGAGGGGCAAGGGGCACGATGGCCTTTACC
AGGGTCTCAGTACAGCCACCAAGGACACCTACGACGCCCTTCACATGCAGGCCCTGCCCCCTC
GCGGCAGCGGCGCCACCAACTTCAGCCTGCTGAAGCAGGCCGGCGACGTGGAGGAAAACCCT
GGCCCCATGTCCAGCAAAGGCTCCGTGGTTCTGGCCTACAGTGGCGGCCTGGACACCTCGTGC
ATCCTCGTGTGGCTGAAGGAACAAGGCTATGACGTCATTGCCTATCTGGCCAACATTGGCCAGA
AGGAAGACTTCGAGGAAGCCAGGAAGAAGGCACTGAAACTTGGGGCCAAAAAGGTGTTCATTGA
GGATGTCAGCAGGGAGTTTGTGGAGGAGTTCATCTGGCCGGCCATCCAGTCCAGCGCACTGTAT
GAGGACCGCTACCTCCTGGGCACCTCTCTTGCCAGGCCCTGCATCGCCCGCAAACAAGTGGAA
ATCGCCCAGCGGGAGGGGGCCAAGTATGTGTCCCACGGCGCCACAGGAAAGGGGAACGATCA
GGTCCGGTTTGAGCTCAGCTGCTACTCACTGGCCCCCCAGATAAAGGTCATTGCTCCCTGGAGG
ATGCCTGAATTCTACAACCGGTTCAAGGGCCGCAATGACCTGATGGAGTACGCAAAGCAACACG
GGATTCCCATCCCGGTCACTCCCAAGAACCCGTGGAGCATGGATGAGAACCTCATGCACATCAG
CTACGAGGCTGGAATCCTGGAGAACCCCAAGAACCAAGCGCCTCCAGGTCTCTACACGAAGACC
CAGGACCCAGCCAAAGCCCCCAACACCCCTGACATTCTCGAGATCGAGTTCAAAAAAGGGGTCC
CTGTGAAGGTGACCAACGTCAAGGATGGCACCACCCACCAGACCTCCTTGGAGCTCTTCATGTA
CCTGAACGAAGTCGCGGGCAAGCATGGCGTGGGCCGTATTGACATCGTGGAGAACCGCTTCAT
TGGAATGAAGTCCCGAGGTATCTACGAGACCCCAGCAGGCACCATCCTTTACCATGCTCATTTAG
ACATCGAGGCCTTCACCATGGACCGGGAAGTGCGCAAAATCAAACAAGGCCTGGGCTTGAAATT
TGCTGAGCTGGTGTATACCGGTTTCTGGCACAGCCCTGAGTGTGAATTTGTCCGCCACTGCATC
GCCAAGTCCCAGGAGCGAGTGGAAGGGAAAGTGCAGGTGTCCGTCCTCAAGGGCCAGGTGTAC
ATCCTCGGCCGGGAGTCCCCACTGTCTCTCTACAATGAGGAGCTGGTGAGCATGAACGTGCAG
GGTGATTATGAGCCAACTGATGCCACCGGGTTCATCAACATCAATTCCCTCAGGCTGAAGGAATA
TCATCGTCTCCAGAGCAAGGTCACTGCCAAAGGAAGCGGAGAGGGCAGAGGAAGTCTGCTAAC
ATGCGGTGACGTCGAGGAGAATCCTGGACCTATGCTGTTTAATCTGAGGATCCTGTTAAACAATG
CAGCTTTTAGAAATGGTCACAACTTCATGGTTCGAAATTTTCGGTGTGGACAACCACTACAAAATA
AAGTGCAGCTGAAGGGCCGTGACCTTCTCACTCTAAAAAACTTTACCGGAGAAGAAATTAAATAT
ATGCTATGGCTATCAGCAGATCTGAAATTTAGGATAAAACAGAAAGGAGAGTATTTGCCTTTATTG
CAAGGGAAGTCCTTAGGCATGATTTTTGAGAAAAGAAGTACTCGAACAAGATTGTCTACAGAAAC
AGGCTTAGCACTTCTGGGAGGACATCCTTGTTTTCTTACCACACAAGATATTCATTTGGGTGTGA
ATGAAAGTCTCACGGACACGGCCCGTGTATTGTCTAGCATGGCAGATGCAGTATTGGCTCGAGT
GTATAAACAATCAGATTTGGACACCCTGGCTAAAGAAGCATCCATCCCAATTATCAATGGGCTGT
CAGATTTGTACCATCCTATCCAGATCCTGGCTGATTACCTCACGCTCCAGGAACACTATAGCTCT
CTGAAAGGTCTTACCCTCAGCTGGATCGGGGATGGGAACAATATCCTGCACTCCATCATGATGA
GCGCAGCGAAATTCGGAATGCACCTTCAGGCAGCTACTCCAAAGGGTTATGAGCCGGATGCTAG
TGTAACCAAGTTGGCAGAGCAGTATGCCAAAGAGAATGGTACCAAGCTGTTGCTGACAAATGATC
CATTGGAAGCAGCGCATGGAGGCAATGTATTAATTACAGACACTTGGATAAGCATGGGACAAGA
AGAGGAGAAGAAAAAGCGGCTCCAGGCTTTCCAAGGTTACCAGGTTACAATGAAGACTGCTAAA
GTTGCTGCCTCTGACTGGACATTTTTACACTGCTTGCCCAGAAAGCCAGAAGAAGTGGATGATGA
AGTCTTTTATTCTCCTCGATCACTAGTGTTCCCAGAGGCAGAAAACAGAAAGTGGACAATCATGG
CTGTCATGGTGTCCCTGCTGACAGATTACTCACCTCAGCTCCAGAAGCCTAAATTTTAA
DNA sequence encoding exemplary CAR of GD2 ASS1 without MP71 vector
Sequence ID NO: 38
ATGCCTCGCGGCTGGACAGCCCTGTGCCTGCTGTCTCTGCTGCCATCCGGCTTCATGAGCCTG
GATAATAACGGCACAGCCACCCCAGAGCTGCCTACACAGGGCACCTTCAGCAATGTGTCCACAA
ACGTGAGCTATCAGGAGACCACAACCCCTTCTACCCTGGGATCCACAAGCCTGCACCCCGTGTC
TCAGCACGGCAACGAAGCCACCACCAACATCACCGAGACCACAGTGAAGTTTACCTCCACCTCT
GTGATTACCTCTGTGTACGGAAATACAAACTCCAGCGTGCAGTCTCAGACATCTGTGATCTCCAC
AGTGTTTACAACACCTGCCAATGTGTCCACCCCAGAGACAACCCTGAAGCCCAGCCTGTCTCCT
GGAAATGTGTCCGATCTGTCTACCACCTCCACCAGCCTGGCCACCTCTCCCACCAAGCCCTATA
CCTCCTCTTCTCCCATCCTGAGCGATATCAAAGCCGAGATCAAATGCAGCGGGATTCGGGAAGT
GAAACTGACACAGGGCATCTGCCTGGAACAGAATAAGACATCCAGCTGCGCCGAGTTTAAGAAA
GATAGAGGAGAGGGACTGGCCAGGGTGCTGTGTGGCGAAGAGCAGGCCGACGCCGATGCCGG
CGCCCAGGTGTGTTCCCTGCTGCTGGCCCAGTCTGAGGTGCGCCCCCAGTGCCTGCTGCTGGT
GCTGGCCAATCGGACAGAAATTAGCAGCAAGCTGCAGCTGATGAAAAAACACCAGAGCGATCTG
AAAAAGCTGGGCATCCTGGACTTTACCGAGCAGGACGTGGCCTCTCACCAGAGCTACAGCCAGA
AAACACTGATCGCCCTGGTGACCAGCGGAGCCCTGCTGGCCGTGCTGGGCATCACCGGATATT
TCCTGATGAATAGGCGCAGCTGGAGCCCCACCGGCGAACGGCTGGAGCTGGAGCCTGTCGACC
GAGTGAAGCAGACCCTGAACTTTGATCTGCTGAAGCTGGCCGGCGACGTGGAGTCCAACCCCG
GGCCAGGGAATATGGCCTTACCAGTGACCGCCTTGCTCCTGCCGCTGGCCTTGCTGCTCCACG
CCGCCAGGCCGGATATTCTGCTCACACAGACCCCACTCTCCCTGCCCGTGTCACTCGGGGATCA
GGCTAGCATTTCTTGCCGCTCATCTCAGTCTCTGGTCCACCGGAATGGGAACACATACCTCCATT
GGTACCTCCAGAAACCTGGACAGAGCCCTAAACTGCTCATCCACAAAGTCTCAAATCGGTTCTCC
GGCGTGCCCGATCGCTTTAGCGGATCCGGATCTGGGACCGACTTCACACTGAAAATCTCACGAG
TGGAGGCTGAGGATCTCGGCGTCTACTTCTGTAGTCAGAGTACCCACGTCCCACCCCTCACCTT
TGGCGCTGGAACAAAACTGGAGCTGAAACGAGCCGATGCTGCTCCTACCGTGTCCATCTTTCCT
GGCTCCGGGGGAGGCGGGAGCGGAGGCGAAGTGAAACTCCAGCAGTCTGGCCCTTCTCTCGT
GGAACCTGGCGCTTCTGTGATGATCTCCTGTAAGGCCTCTGGATCTTCCTTTACCGGCTACAACA
TGAACTGGGTCCGGCAGAACATTGGCAAATCCCTGGAATGGATTGGCGCCATCGATCCTTACTA
CGGCGGCACATCATACAATCAGAAATTCAAGGGGCGAGCAACACTCACTGTCGACAAATCTTCAT
CCACCGCCTACATGCACCTGAAATCTCTCACATCCGAGGATAGTGCTGTCTACTACTGTGTCTCT
GGCATGGAATACTGGGGACAGGGAACTTCTGTCACCGTGTCTAGTGCCAAAACCACACCTCCCT
CCGTGTACGGACGAGTCACTGTCTCATCTGCTGAACCAAAATCCTGTGACAAAACACACACATGC
CCACCTTGTCCTGCCCCTGAACTGCTCGGCGGACCTTCCGTCTTTCTGTTTCCCCCCAAACCCAA
GGATACACTCATGATTTCTAGGACCCCCGAAGTCACTTGTGTCGTGGTCGATGTGTCTCACGAG
GATCCTGAAGTGAAATTCAACTGGTACGTGGACGGAGTCGAGGTCCACAATGCCAAAACAAAAC
CCCGGGAGGAACAGTACAATAGCACCTACCGAGTCGTGTCCGTGCTCACCGTCCTCCATCAGGA
TTGGCTGAACGGCAAAGAGTACAAGTGTAAAGTGAGTAACAAGGCTCTCCCCGCTCCTATTGAAA
AAACCATCTCAAAAGCAAAAGGCCAGCCTAGGGAGCCTCAGGTCTACACACTGCCACCCTCACG
GGACGAACTCACCAAAAATCAGGTGTCCCTCACTTGCCTGGTGAAAGGCTTCTACCCTTCCGATA
TCGCTGTGGAATGGGAGTCAAATGGGCAGCCCGAAAACAACTACAAAACAACCCCCCCTGTGCT
CGATTCCGATGGCTCTTTTTTCCTGTACTCCAAACTCACCGTGGACAAATCACGCTGGCAGCAGG
GGAATGTCTTTTCTTGCTCCGTGATGCACGAGGCCCTCCACAATCATTACACCCAGAAATCCCTC
TCACTCTCACCCGGCAAAAAGGACCCTAAAACCACGACGCCAGCGCCGCGACCACCAACACCG
GCGCCCACCATCGCGTCGCAGCCCCTGTCCCTGCGCCCAGAGGCGTGCCGGCCAGCGGCGGG
GGGCGCAGTGCACACGAGGGGGCTGGACTTCGCCTGTGATATCTACATCTGGGCGCCCTTGGC
CGGGACTTGTGGGGTCCTTCTCCTGTCACTGGTTATCACCCTTTACTGCAAACGGGGCAGAAAG
AAACTCCTGTATATATTCAAACAACCATTTATGAGACCAGTACAAACTACTCAAGAGGAAGATGGC
TGTAGCTGCCGATTTCCAGAAGAAGAAGAAGGAGGATGTGAACTGAGAGTGAAGTTCAGCAGGA
GCGCAGACGCCCCCGCGTACAAGCAGGGCCAGAACCAGCTCTATAACGAGCTCAATCTAGGAC
GAAGAGAGGAGTACGATGTTTTGGACAAGAGACGTGGCCGGGACCCTGAGATGGGGGGAAAGC
CGAGAAGGAAGAACCCTCAGGAAGGCCTGTACAATGAACTGCAGAAAGATAAGATGGCGGAGG
CCTACAGTGAGATTGGGATGAAAGGCGAGCGCCGGAGGGGCAAGGGGCACGATGGCCTTTACC
AGGGTCTCAGTACAGCCACCAAGGACACCTACGACGCCCTTCACATGCAGGCCCTGCCCCCTC
GCGGCAGCGGCGCCACCAACTTCAGCCTGCTGAAGCAGGCCGGCGACGTGGAGGAAAACCCT
GGCCCCATGTCCAGCAAAGGCTCCGTGGTTCTGGCCTACAGTGGCGGCCTGGACACCTCGTGC
ATCCTCGTGTGGCTGAAGGAACAAGGCTATGACGTCATTGCCTATCTGGCCAACATTGGCCAGA
AGGAAGACTTCGAGGAAGCCAGGAAGAAGGCACTGAAACTTGGGGCCAAAAAGGTGTTCATTGA
GGATGTCAGCAGGGAGTTTGTGGAGGAGTTCATCTGGCCGGCCATCCAGTCCAGCGCACTGTAT
GAGGACCGCTACCTCCTGGGCACCTCTCTTGCCAGGCCCTGCATCGCCCGCAAACAAGTGGAA
ATCGCCCAGCGGGAGGGGGCCAAGTATGTGTCCCACGGCGCCACAGGAAAGGGGAACGATCA
GGTCCGGTTTGAGCTCAGCTGCTACTCACTGGCCCCCCAGATAAAGGTCATTGCTCCCTGGAGG
ATGCCTGAATTCTACAACCGGTTCAAGGGCCGCAATGACCTGATGGAGTACGCAAAGCAACACG
GGATTCCCATCCCGGTCACTCCCAAGAACCCGTGGAGCATGGATGAGAACCTCATGCACATCAG
CTACGAGGCTGGAATCCTGGAGAACCCCAAGAACCAAGCGCCTCCAGGTCTCTACACGAAGACC
CAGGACCCAGCCAAAGCCCCCAACACCCCTGACATTCTCGAGATCGAGTTCAAAAAAGGGGTCC
CTGTGAAGGTGACCAACGTCAAGGATGGCACCACCCACCAGACCTCCTTGGAGCTCTTCATGTA
CCTGAACGAAGTCGCGGGCAAGCATGGCGTGGGCCGTATTGACATCGTGGAGAACCGCTTCAT
TGGAATGAAGTCCCGAGGTATCTACGAGACCCCAGCAGGCACCATCCTTTACCATGCTCATTTAG
ACATCGAGGCCTTCACCATGGACCGGGAAGTGCGCAAAATCAAACAAGGCCTGGGCTTGAAATT
TGCTGAGCTGGTGTATACCGGTTTCTGGCACAGCCCTGAGTGTGAATTTGTCCGCCACTGCATC
GCCAAGTCCCAGGAGCGAGTGGAAGGGAAAGTGCAGGTGTCCGTCCTCAAGGGCCAGGTGTAC
ATCCTCGGCCGGGAGTCCCCACTGTCTCTCTACAATGAGGAGCTGGTGAGCATGAACGTGCAG
GGTGATTATGAGCCAACTGATGCCACCGGGTTCATCAACATCAATTCCCTCAGGCTGAAGGAATA
TCATCGTCTCCAGAGCAAGGTCACTGCCAAATAA
DNA sequence encoding exemplary CAR of GD2 OTC without MP71 vector
Sequence ID NO: 39
ATGCCTCGCGGCTGGACAGCCCTGTGCCTGCTGTCTCTGCTGCCATCCGGCTTCATGAGCCTG
GATAATAACGGCACAGCCACCCCAGAGCTGCCTACACAGGGCACCTTCAGCAATGTGTCCACAA
ACGTGAGCTATCAGGAGACCACAACCCCTTCTACCCTGGGATCCACAAGCCTGCACCCCGTGTC
TCAGCACGGCAACGAAGCCACCACCAACATCACCGAGACCACAGTGAAGTTTACCTCCACCTCT
GTGATTACCTCTGTGTACGGAAATACAAACTCCAGCGTGCAGTCTCAGACATCTGTGATCTCCAC
AGTGTTTACAACACCTGCCAATGTGTCCACCCCAGAGACAACCCTGAAGCCCAGCCTGTCTCCT
GGAAATGTGTCCGATCTGTCTACCACCTCCACCAGCCTGGCCACCTCTCCCACCAAGCCCTATA
CCTCCTCTTCTCCCATCCTGAGCGATATCAAAGCCGAGATCAAATGCAGCGGGATTCGGGAAGT
GAAACTGACACAGGGCATCTGCCTGGAACAGAATAAGACATCCAGCTGCGCCGAGTTTAAGAAA
GATAGAGGAGAGGGACTGGCCAGGGTGCTGTGTGGCGAAGAGCAGGCCGACGCCGATGCCGG
CGCCCAGGTGTGTTCCCTGCTGCTGGCCCAGTCTGAGGTGCGCCCCCAGTGCCTGCTGCTGGT
GCTGGCCAATCGGACAGAAATTAGCAGCAAGCTGCAGCTGATGAAAAAACACCAGAGCGATCTG
AAAAAGCTGGGCATCCTGGACTTTACCGAGCAGGACGTGGCCTCTCACCAGAGCTACAGCCAGA
AAACACTGATCGCCCTGGTGACCAGCGGAGCCCTGCTGGCCGTGCTGGGCATCACCGGATATT
TCCTGATGAATAGGCGCAGCTGGAGCCCCACCGGCGAACGGCTGGAGCTGGAGCCTGTCGACC
GAGTGAAGCAGACCCTGAACTTTGATCTGCTGAAGCTGGCCGGCGACGTGGAGTCCAACCCCG
GGCCAGGGAATATGGCCTTACCAGTGACCGCCTTGCTCCTGCCGCTGGCCTTGCTGCTCCACG
CCGCCAGGCCGGATATTCTGCTCACACAGACCCCACTCTCCCTGCCCGTGTCACTCGGGGATCA
GGCTAGCATTTCTTGCCGCTCATCTCAGTCTCTGGTCCACCGGAATGGGAACACATACCTCCATT
GGTACCTCCAGAAACCTGGACAGAGCCCTAAACTGCTCATCCACAAAGTCTCAAATCGGTTCTCC
GGCGTGCCCGATCGCTTTAGCGGATCCGGATCTGGGACCGACTTCACACTGAAAATCTCACGAG
TGGAGGCTGAGGATCTCGGCGTCTACTTCTGTAGTCAGAGTACCCACGTCCCACCCCTCACCTT
TGGCGCTGGAACAAAACTGGAGCTGAAACGAGCCGATGCTGCTCCTACCGTGTCCATCTTTCCT
GGCTCCGGGGGAGGCGGGAGCGGAGGCGAAGTGAAACTCCAGCAGTCTGGCCCTTCTCTCGT
GGAACCTGGCGCTTCTGTGATGATCTCCTGTAAGGCCTCTGGATCTTCCTTTACCGGCTACAACA
TGAACTGGGTCCGGCAGAACATTGGCAAATCCCTGGAATGGATTGGCGCCATCGATCCTTACTA
CGGCGGCACATCATACAATCAGAAATTCAAGGGGCGAGCAACACTCACTGTCGACAAATCTTCAT
CCACCGCCTACATGCACCTGAAATCTCTCACATCCGAGGATAGTGCTGTCTACTACTGTGTCTCT
GGCATGGAATACTGGGGACAGGGAACTTCTGTCACCGTGTCTAGTGCCAAAACCACACCTCCCT
CCGTGTACGGACGAGTCACTGTCTCATCTGCTGAACCAAAATCCTGTGACAAAACACACACATGC
CCACCTTGTCCTGCCCCTGAACTGCTCGGCGGACCTTCCGTCTTTCTGTTTCCCCCCAAACCCAA
GGATACACTCATGATTTCTAGGACCCCCGAAGTCACTTGTGTCGTGGTCGATGTGTCTCACGAG
GATCCTGAAGTGAAATTCAACTGGTACGTGGACGGAGTCGAGGTCCACAATGCCAAAACAAAAC
CCCGGGAGGAACAGTACAATAGCACCTACCGAGTCGTGTCCGTGCTCACCGTCCTCCATCAGGA
TTGGCTGAACGGCAAAGAGTACAAGTGTAAAGTGAGTAACAAGGCTCTCCCCGCTCCTATTGAAA
AAACCATCTCAAAAGCAAAAGGCCAGCCTAGGGAGCCTCAGGTCTACACACTGCCACCCTCACG
GGACGAACTCACCAAAAATCAGGTGTCCCTCACTTGCCTGGTGAAAGGCTTCTACCCTTCCGATA
TCGCTGTGGAATGGGAGTCAAATGGGCAGCCCGAAAACAACTACAAAACAACCCCCCCTGTGCT
CGATTCCGATGGCTCTTTTTTCCTGTACTCCAAACTCACCGTGGACAAATCACGCTGGCAGCAGG
GGAATGTCTTTTCTTGCTCCGTGATGCACGAGGCCCTCCACAATCATTACACCCAGAAATCCCTC
TCACTCTCACCCGGCAAAAAGGACCCTAAAACCACGACGCCAGCGCCGCGACCACCAACACCG
GCGCCCACCATCGCGTCGCAGCCCCTGTCCCTGCGCCCAGAGGCGTGCCGGCCAGCGGCGGG
GGGCGCAGTGCACACGAGGGGGCTGGACTTCGCCTGTGATTTTTGGGTGCTGGTGGTGGTTGG
TGGAGTCCTGGCTTGCTATAGCTTGCTAGTAACAGTGGCCTTTATTATTTTCTGGGTGAGGAGTA
AGAGGAGCAGGCTCCTGCACAGTGACTACATGAACATGACTCCCCGCCGCCCCGGGCCCACCC
GCAAGCATTACCAGCCCTATGCCCCACCTCGCGACTTCGCAGCCTATCGCTCCAGAGTGAAGTT
CAGCAGGAGCGCAGACGCCCCCGCGTACAAGCAGGGCCAGAACCAGCTCTATAACGAGCTCAA
TCTAGGACGAAGAGAGGAGTACGATGTTTTGGACAAGAGACGTGGCCGGGACCCTGAGATGGG
GGGAAAGCCGAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGCACGATGG
CCTTTACCAGGGTCTCAGTACAGCCACCAAGGACACCTACGACGCCCTTCACATGCAGGCCCTG
CCCCCTCGCGGCAGCGGCGCCACCAACTTCAGCCTGCTGAAGCAGGCCGGCGACGTGGAGGA
AAACCCTGGCCCCATGCTGTTTAATCTGAGGATCCTGTTAAACAATGCAGCTTTTAGAAATGGTC
ACAACTTCATGGTTCGAAATTTTCGGTGTGGACAACCACTACAAAATAAAGTGCAGCTGAAGGGC
CGTGACCTTCTCACTCTAAAAAACTTTACCGGAGAAGAAATTAAATATATGCTATGGCTATCAGCA
GATCTGAAATTTAGGATAAAACAGAAAGGAGAGTATTTGCCTTTATTGCAAGGGAAGTCCTTAGG
CATGATTTTTGAGAAAAGAAGTACTCGAACAAGATTGTCTACAGAAACAGGCTTAGCACTTCTGG
GAGGACATCCTTGTTTTCTTACCACACAAGATATTCATTTGGGTGTGAATGAAAGTCTCACGGAC
ACGGCCCGTGTATTGTCTAGCATGGCAGATGCAGTATTGGCTCGAGTGTATAAACAATCAGATTT
GGACACCCTGGCTAAAGAAGCATCCATCCCAATTATCAATGGGCTGTCAGATTTGTACCATCCTA
TCCAGATCCTGGCTGATTACCTCACGCTCCAGGAACACTATAGCTCTCTGAAAGGTCTTACCCTC
AGCTGGATCGGGGATGGGAACAATATCCTGCACTCCATCATGATGAGCGCAGCGAAATTCGGAA
TGCACCTTCAGGCAGCTACTCCAAAGGGTTATGAGCCGGATGCTAGTGTAACCAAGTTGGCAGA
GCAGTATGCCAAAGAGAATGGTACCAAGCTGTTGCTGACAAATGATCCATTGGAAGCAGCGCAT
GGAGGCAATGTATTAATTACAGACACTTGGATAAGCATGGGACAAGAAGAGGAGAAGAAAAAGC
GGCTCCAGGCTTTCCAAGGTTACCAGGTTACAATGAAGACTGCTAAAGTTGCTGCCTCTGACTG
GACATTTTTACACTGCTTGCCCAGAAAGCCAGAAGAAGTGGATGATGAAGTCTTTTATTCTCCTC
GATCACTAGTGTTCCCAGAGGCAGAAAACAGAAAGTGGACAATCATGGCTGTCATGGTGTCCCT
GCTGACAGATTACTCACCTCAGCTCCAGAAGCCTAAATTTTAA
Alternative CD3z
Sequence ID NO: 40
AGAGTGAAGTTCAGCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAGACGTGGCCGGGAC
CCTGAGATGGGGGGAAAGCCGCAGAGAAGGAAGAACCCTCAGGAAGGCCTGTACAATGAACTG
CAGAAAGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGACGCCCTT
CACATGCAGGCCCTGCCCCCTCGCTAA
DNA encoding an alternative EGFRvIII target binding moiety (EGFRvIII
scFv sequence derived from MR1 antibody)
Sequence ID No: 41
atggactggatttggcgcatccttttccttgtcggcgctgctaccggcgcgcattctcaggtacaact
ccagcagtctgggggaggcttagtgaagcctggagcgtctctgaaactctcctgtgtaacctctggat
tcactttcagaaaatttggcatgtcttgggttcgccagactagtgacaagaggctggaatgggtcgca
tccattagtactggcggttataacacgtactattcagacaatgtaaagggccgattcaccatctccag
agagaatgccaagaacaccctgtacctgcaaatgagtagtctgaagtctgaggacacggccttgtatt
actgtacaagaggctattctagtacctcttatgctatggactactggggccaagggaccacggtcacc
gtctcctcaagtggaggcggttcaggcggaggtggctctggcggtggcggatcggacatcgagctcac
tcagtctccagcatccctgtccgtggctacaggagaaaaagtcactatcagatgcatgaccagcactg
atattgatgatgatatgaactggtaccagcagaagccaggggaaccccctaagttccttatttcagaa
ggcaatactcttcggccgggagtcccatcccgattttccagcagtggcactggcacagattttgtttt
tacaattgaaaacacactctcggaagatgttggagattactactgtttgcaaagctttaacgtgcctc
ttacattcggtgatggcaccaagcttgaaaaagctcta
amino acid sequence of an exemplary CAR of EGFRvIII (employing
the alternative EGFRvIII target binding moiety encoded by SEQ ID NO: 41)
including an ASS-1 domain
Sequence ID No: 42
MPRGWTALCLLSLLPSGFMSLDNNGTATPELPTQGTFSNVSTNVSYQETTTPSTLGSTSLHPVSQHGNEATTNITE
TTVKFTSTSVITSVYGNTNSSVQSQTSVISTVFTTPANVSTPETTLKPSLSPGNVSDLSTTSTSLATSPTKPYTSSSPILSD
IKAEIKCSGIREVKLTQGICLEQNKTSSCAEFKKDRGEGLARVLCGEEQADADAGAQVCSLLLAQSEVRPQCLLLVLA
NRTEISSKLQLMKKHQSDLKKLGILDFTEQDVASHQSYSQKTLIALVTSGALLAVLGITGYFLMNRRSWSPTGERLEL
EPVDRVKQTLNFDLLKLAGDVESNPGPGNMALPVTALLLPLALLLHAARPMDWIWRILFLVGAATGAHSQVQLQ
QSGGGLVKPGASLKLSCVTSGFTFRKFGMSWVRQTSDKRLEWVASISTGGYNTYYSDNVKGRFTISRENAKNTLYL
QMSSLKSEDTALYYCTRGYSSTSYAMDYWGQGTTVTVSSSGGGSGGGGSGGGGSDIELTQSPASLSVATGEKVTI
RCMTSTDIDDDMNWYQQKPGEPPKFLISEGNTLRPGVPSRFSSSGTGTDFVFTIENTLSEDVGDYYCLQSFNVPLTF
GDGTKLEKALTTTPAPRPPTPAPTIASQPLSLRPEACRPAAGGAVHTRGLDFACDIYIWAPLAGTCGVLLLSLVITLYC
KRGRKKLLYIFKQPFMRPVQTTQEEDGCSCRFPEEEEGGCELRVKFSRSADAPAYKQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPRGSGATNFSLLKQAGDVEENPGPMSSKGSVVLAYSGGLDTSCILVWLKEQGYDVIAYLANIGQKEDFEEAR
KKALKLGAKKVFIEDVSREFVEEFIWPAIQSSALYEDRYLLGTSLARPCIARKQVEIAQREGAKYVSHGATGKGNDQV
RFELSCYSLAPQIKVIAPWRMPEFYNRFKGRNDLMEYAKQHGIPIPVTPKNPWSMDENLMHISYEAGILENPKNQ
APPGLYTKTQDPAKAPNTPDILEIEFKKGVPVKVTNVKDGTTHQTSLELFMYLNEVAGKHGVGRIDIVENRFIGMKS
RGIYETPAGTILYHAHLDIEAFTMDREVRKIKQGLGLKFAELVYTGFWHSPECEFVRHCIAKSQERVEGKVQVSVLKG
QVYILGRESPLSLYNEELVSMNVQGDYEPTDATGFININSLRLKEYHRLQSKVTAK
amino acid sequence of an exemplary CAR of EGFRvIII (employing
the alternative EGFRvIII target binding moiety encoded by SEQ ID NO: 41) including an OTC
domain
Sequence ID No: 43
MPRGWTALCLLSLLPSGFMSLDNNGTATPELPTQGTFSNVSTNVSYQETTTPSTLGSTSLHPVSQHGNEATTNITE
TTVKFTSTSVITSVYGNTNSSVQSQTSVISTVFTTPANVSTPETTLKPSLSPGNVSDLSTTSTSLATSPTKPYTSSSPILSD
IKAEIKCSGIREVKLTQGICLEQNKTSSCAEFKKDRGEGLARVLCGEEQADADAGAQVCSLLLAQSEVRPQCLLLVLA
NRTEISSKLQLMKKHQSDLKKLGILDFTEQDVASHQSYSQKTLIALVTSGALLAVLGITGYFLMNRRSWSPTGERLEL
EPVDRVKQTLNFDLLKLAGDVESNPGPGNMALPVTALLLPLALLLHAARPMDWIWRILFLVGAATGAHSQVQLQ
QSGGGLVKPGASLKLSCVTSGFTFRKFGMSWVRQTSDKRLEWVASISTGGYNTYYSDNVKGRFTISRENAKNTLYL
QMSSLKSEDTALYYCTRGYSSTSYAMDYWGQGTTVTVSSSGGGSGGGGSGGGGSDIELTQSPASLSVATGEKVTI
RCMTSTDIDDDMNWYQQKPGEPPKFLISEGNTLRPGVPSRFSSSGTGTDFVFTIENTLSEDVGDYYCLQSFNVPLTF
GDGTKLEKALTTTPAPRPPTPAPTIASQPLSLRPEACRPAAGGAVHTRGLDFACDIYIWAPLAGTCGVLLLSLVITLYC
KRGRKKLLYIFKQPFMRPVQTTQEEDGCSCRFPEEEEGGCELRVKFSRSADAPAYKQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPRGSGATNFSLLKQAGDVEENPGPMLFNLRILLNNAAFRNGHNFMVRNFRCGQPLQNKVQLKGRDLLTLK
NFTGEEIKYMLWLSADLKFRIKQKGEYLPLLQGKSLGMIFEKRSTRTRLSTETGLALLGGHPCFLTTQDIHLGVNESLT
DTARVLSSMADAVLARVYKQSDLDTLAKEASIPIINGLSDLYHPIQILADYLTLQEHYSSLKGLTLSWIGDGNNILHSI
MMSAAKFGMHLQAATPKGYEPDASVTKLAEQYAKENGTKLLLTNDPLEAAHGGNVLITDTWISMGQEEEKKKRL
QAFQGYQVTMKTAKVAASDWTFLHCLPRKPEEVDDEVFYSPRSLVFPEAENRKWTIMAVMVSLLTDYSPQLQKP
KF
amino acid sequence of an exemplary CAR of EGFRvIII (employing
the alternative EGFRvIII target binding moiety encoded by SEQ ID NO: 41)
including an ASS-1 domain and an OTC domain
Sequence ID No: 44
MPRGWTALCLLSLLPSGFMSLDNNGTATPELPTQGTFSNVSTNVSYQETTTPSTLGSTSLHPVSQHGNEATTNITE
TTVKFTSTSVITSVYGNTNSSVQSQTSVISTVFTTPANVSTPETTLKPSLSPGNVSDLSTTSTSLATSPTKPYTSSSPILSD
IKAEIKCSGIREVKLTQGICLEQNKTSSCAEFKKDRGEGLARVLCGEEQADADAGAQVCSLLLAQSEVRPQCLLLVLA
NRTEISSKLQLMKKHQSDLKKLGILDFTEQDVASHQSYSQKTLIALVTSGALLAVLGITGYFLMNRRSWSPTGERLEL
EPVDRVKQTLNFDLLKLAGDVESNPGPGNMALPVTALLLPLALLLHAARPMDWIWRILFLVGAATGAHSQVQLQ
QSGGGLVKPGASLKLSCVTSGFTFRKFGMSWVRQTSDKRLEWVASISTGGYNTYYSDNVKGRFTISRENAKNTLYL
QMSSLKSEDTALYYCTRGYSSTSYAMDYWGQGTTVTVSSSGGGSGGGGSGGGGSDIELTQSPASLSVATGEKVTI
RCMTSTDIDDDMNWYQQKPGEPPKFLISEGNTLRPGVPSRFSSSGTGTDFVFTIENTLSEDVGDYYCLQSFNVPLTF
GDGTKLEKALTTTPAPRPPTPAPTIASQPLSLRPEACRPAAGGAVHTRGLDFACDIYIWAPLAGTCGVLLLSLVITLYC
KRGRKKLLYIFKQPFMRPVQTTQEEDGCSCRFPEEEEGGCELRVKFSRSADAPAYKQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPRGSGATNFSLLKQAGDVEENPGPMSSKGSVVLAYSGGLDTSCILVWLKEQGYDVIAYLANIGQKEDFEEAR
KKALKLGAKKVFIEDVSREFVEEFIWPAIQSSALYEDRYLLGTSLARPCIARKQVEIAQREGAKYVSHGATGKGNDQV
RFELSCYSLAPQIKVIAPWRMPEFYNRFKGRNDLMEYAKQHGIPIPVTPKNPWSMDENLMHISYEAGILENPKNQ
APPGLYTKTQDPAKAPNTPDILEIEFKKGVPVKVTNVKDGTTHQTSLELFMYLNEVAGKHGVGRIDIVENRFIGMKS
RGIYETPAGTILYHAHLDIEAFTMDREVRKIKQGLGLKFAELVYTGFWHSPECEFVRHCIAKSQERVEGKVQVSVLKG
QVYILGRESPLSLYNEELVSMNVQGDYEPTDATGFININSLRLKEYHRLQSKVTAKGSGEGRGSLLTCGDVEENPGP
MLFNLRILLNNAAFRNGHNFMVRNFRCGQPLQNKVQLKGRDLLTLKNFTGEEIKYMLWLSADLKFRIKQKGEYLPL
LQGKSLGMIFEKRSTRTRLSTETGLALLGGHPCFLTTQDIHLGVNESLTDTARVLSSMADAVLARVYKQSDLDTLAKE
ASIPIINGLSDLYHPIQILADYLTLQEHYSSLKGLTLSWIGDGNNILHSIMMSAAKFGMHLQAATPKGYEPDASVTKL
AEQYAKENGTKLLLTNDPLEAAHGGNVLITDTWISMGQEEEKKKRLQAFQGYQVTMKTAKVAASDWTFLHCLPR
KPEEVDDEVFYSPRSLVFPEAENRKWTIMAVMVSLLTDYSPQLQKPKF

1. A fusion target-binding protein comprising a target binding moiety, an intracellular signalling region and a domain that promotes synthesis of arginine or an arginine precursor.

2. The fusion target-binding protein according to claim 1, wherein the domain that promotes synthesis of arginine or an arginine precursor comprises an enzyme domain.

3. The fusion target-binding protein according to claim 2, wherein the enzyme domain is selected from the group consisting of: an ASS-1 domain; an OTC domain; an ASL domain; an OCD1 domain; an ArgG domain; an ArgH domain; and an ArgF domain.

4. The fusion target-binding protein according to claim 3, wherein the ASS-1 domain comprises the amino acid sequence of SEQ ID NO. 1.

5. The fusion target-binding protein according to claim 3, wherein the OTC domain comprises the amino acid sequence of SEQ ID NO. 2, the ASL domain comprises the amino acid sequence encoded by SEQ ID NO. 30, the ODC1 domain comprises the amino acid sequence encoded by SEQ ID NO. 31, the ArgG domain comprises the amino acid sequence encoded by SEQ ID NO. 32, the ArgH domain comprises the amino acid sequence encoded by SEQ ID NO. 33, and the ArgF domain comprises the amino acid sequence encoded by SEQ ID NO. 34.

6-10. (canceled)

11. A fusion target-binding protein comprising a target binding moiety, an intracellular signalling region and a domain that promotes synthesis of tryptophan or a tryptophan precursor.

12. The fusion target-binding protein according to claim 11, wherein the domain that promotes synthesis of tryptophan or a tryptophan precursor comprises an enzyme domain.

13. The fusion target-binding protein according to claim 11, wherein the enzyme domain is selected from the group consisting of: an TRP5 domain; and an IDO domain, and wherein the TRP5 domain comprises the amino acid sequence encoded by SEQ ID NO. 35, and the IDO domain comprises the amino acid sequence encoded by SEQ ID NO. 36.

14. (canceled)

15. (canceled)

16. The fusion target-binding protein according to claim 1, wherein the target binding moiety is selected from the group consisting of: a GD2 target binding moiety; a CD33 target binding moiety; a mesothelin target binding moiety; and an EGFRvIII target binding moiety.

17. The fusion target-binding protein according to claim 16, wherein the GD2 target binding moiety comprises the amino acid sequence of SEQ ID NO. 3, the CD33 target binding moiety comprises the amino acid sequence of SEQ ID NO. 4, the mesothelin target binding moiety comprises the amino acid sequence of SEQ ID NO. 5, the EGFRvIII target binding moiety comprises the amino acid sequence of SEQ ID NO. 6.

18-20. (canceled)

21. The fusion target-binding protein according to claim 1, wherein the intracellular signalling region comprises a region selected from the group consisting of: a 4-1BB signalling region; an OX-40 signalling region; a CD28 signalling region; an ICOS signalling region; and a CD3ζ signalling region.

22. The fusion target-binding protein according to claim 21, wherein the intracellular signalling region comprises a 4-1BB signalling region.

23. The fusion target-binding protein according to claim 21, wherein the 4-1BB intracellular signalling region comprises the amino acid sequence of SEQ ID NO. 7.

24. The fusion target-binding protein according 21, wherein the intracellular signalling region comprises a CD3ζ domain.

25. The fusion target-binding protein according to claim 24, wherein the CD3 domain comprises the amino acid sequence of SEQ ID NO: 11 or SEQ ID NO: 40.

26. The fusion target-binding protein according to claim 21, comprising both a 4-1BB domain and a CD3ζ domain.

27. The fusion target-binding protein according to claim 1, comprising an amino acid sequence selected from the group consisting of SEQ ID NOs: 12 to 23.

28. (canceled)

29. (canceled)

30. A cell comprising a fusion target-binding protein comprising a target binding moiety, an intracellular signalling region and a domain that promotes synthesis of arginine or an arginine precursor, wherein the fusion target-binding protein is as defined in claim 1.

31. (canceled)

32. A cell comprising a fusion target-binding protein comprising a target binding moiety, an intracellular signalling region and a domain that promotes synthesis of tryptophan or a tryptophan precursor, wherein the fusion target-binding protein is as defined in claim 11.

33-35. (canceled)

36. A nucleic acid encoding a fusion target-binding protein comprising a target binding moiety, an intracellular signalling region and a domain that promotes synthesis of arginine or an arginine precursor, wherein the fusion-target binding protein is as defined in claim 1.

37. (canceled)

38. A nucleic acid according to claim 36, comprising the DNA sequence of SEQ ID NO: 38, SEQ ID NO: 39; or SEQ ID NO: 37.

39. A nucleic acid encoding a fusion target-binding protein comprising a target binding moiety, an intracellular signalling region and a domain that promotes synthesis of tryptophan or a tryptophan precursor, wherein the fusion target-binding protein is as defined in claim 11.

40-43. (canceled)

43. A pharmaceutical composition comprising a fusion target-binding protein according claim 1.

44. (canceled)

45. (canceled)

46. A method of treating a condition in a subject in need thereof, the method comprising providing the subject with a fusion target-binding protein according to claim 1.

47. The method according to claim 45, wherein the fusion target-binding protein is provided by cellular expression of a nucleic acid sequence according to claim 37.

48. A method of treating a condition in a subject in need thereof, the method comprising providing the subject with a cell according to claim 30.

49. A method of treating a condition in a subject in need thereof, the method comprising providing the subject with a nucleic acid according to claim 36.

50. The method according to claim 46, in the treatment of cancer.

51. The method according to claim 46, in the treatment of viral infection.

52. The fusion target-binding protein of claim 1, wherein the fusion target-binding protein is a chimeric antigen receptor (CAR) protein.
